(19) United States
(12) Patent Application Publication
(10) Pub. No.: US 2007/0128707 A1
(43) Pub. Date: Jun. 7, 2007

(54) Method for making metal oxides

(75) Inventors: Gregory L. Rorrer, Corvallis, OR (US); Chih-Hung Chang, Corvallis, OR (US); Clayton Jeffryes, Corvallis, OR (US); Tian Qin, Corvallis, OR (US); Doo-hyoung Lee, Corvallis, OR (US); Debra Gale, Corvallis, OR (US)

Correspondence Address: KLARQUIST SPARKMAN, LLP, 121 SW SALMON STREET, SUITE 1600, PORTLAND, OR, 97204, US

(73) Assignee: Oregon State University

(21) Appl. No.: 11/595,479

(22) Filed: Nov. 10, 2006

Related U.S. Application Data
(60) Provisional application No. 60/735,350, filed on Nov. 10, 2005.

(51) Int. Cl. C12P 3/00; G02F 1/03

(52) U.S. Cl. 435/168; 359/245

(57) ABSTRACT

Embodiments of a method for making metal oxides or mixed metal oxides are disclosed. One representative embodiment comprises selecting a substrate for making the metal oxide or mixed metal oxide, contacting a biological system with the substrate material for an effective period of time, and optionally isolating the metal oxide or mixed metal oxide from the biological system. The biological system can be a living organism, or proteins derived from the living organism. Working embodiments used microalgae, specifically diatoms of the class Bacillariophycae, including marine Pennate diatoms, such as Nitzschia and/or Pinnularia diatoms, and Centric diatoms, such as Cyclotella species. The method also can involve post processing a primary or secondary material produced biologically. The metal oxides can be used for a variety of applications, including optoelectronic devices, such as memory devices, data storage devices, detectors, sensors, lasers, emitters, light-emitting diodes, optical computing architectures, photonics, imaging, photovoltaics, and administering therapeutic.

### CROSS REFERENCE TO RELATED APPLICATIONS

This application claims the benefit of the earlier filing date of U.S. provisional patent application No. 60/735,350, entitled “Method for Making Metal Oxides,” which was filed on Nov. 10, 2005, and is incorporated herein by reference in its entirety.

### ACKNOWLEDGMENT OF GOVERNMENT SUPPORT

Embodiments of the present disclosure may have been developed using federal funding under NSF grants, including NSF grant number 0210384 and NSF grant number 0400648. The United States government may have rights in the invention.

### FIELD

The present disclosure concerns embodiments of a method for making oxide materials, particularly metal oxide materials, even more particularly luminescent and/or semiconductor metal oxides, oxide materials made by the method, and devices and methods that utilize the oxide materials.

### BACKGROUND

A. Optoelectronic Semiconductor Materials and Devices

Nanostructured metal oxide semiconductor materials possess novel optical and electronic properties that can be tuned by manipulating the dimensions of the composite nanophases in the 1-100 nm size range. Nanomaterials possessing these properties have a host of potential applications, particularly for the fabrication of optoelectronic materials and devices, including memory and data storage, capacitors, detectors, sensors, lasers, emitters, light-emitting diodes, optical computing architectures, photonics (e.g. waveguides), imaging devices, photovoltaics (e.g. solar cells) and photocatalysts.

State-of-the-art technologies for producing nanopatterned semiconductor materials and devices usually involve cumbersome chemical synthesis methods or exotic and energy-intensive processes that generate a significant amount of waste. For example, nanostructured Si—Ge semiconductor composite materials are made using processes operating at high temperature and near vacuum conditions. K. Rajeshwar, N. R. de Tacconi, and C. R. Chenthamarakshan, Semiconductor-based composite materials: Preparation, properties, and performance, Chem. Mater. 13, 2765 (2001). Furthermore, these technologies are “top down,” meaning that large and sophisticated equipment must be used to fabricate microscale and nanoscale features into the device structures. Conventional photolithography used in the manufacture of silicon-based semiconductor devices, e.g. highly-integrated (VLSI) transistors, is an example of a top-down process.

B. Diatoms

Marine organisms are inspirational sources of novel inorganic materials exquisitely ordered at the nano- and microscale. Diatoms, for example,. are a prolific class of microalgae (the Bacillariophyceae) utilizing controlled nanostructured silica production. See, N. Kröger et al., Polycationic peptides from diatom biosilica that direct silica nanosphere formation, Science 286, 1129 (1999). Their cell walls consist of silica nanospheres that are assembled to form exoskeletons with intricate microscale features. Several investigators have proposed “silicon biotechnology” applications for this biological process. See, for example, D. E. Morse, Silicon biotechnology: Harnessing biological silica production to construct new materials. TIBTECH 17, 230 (1999); and J. Parkinson and R. Gordon, Beyond micromachining: The potential of diatoms. TIBTECH 17, 190 (1999).

The extent to which metal oxide products others than those normally made by an organism, such as a diatom, can be made biologically also is unknown. Soluble germanium, for example, inhibits cell division in diatoms. J. Lewin, Silicon metabolism in diatoms. V. Germanium dioxide, a specific inhibitor of diatom growth. Phycologia 6, 1 (1966). Limited previous work using ⁶⁸Ge radioisotope labeling showed that the heterotrophic marine diatom Nitzschia alba rapidly assimilated soluble germanium. See, F. Azam, B. B. Hemmingsen, and B. E. Volcani, Germanium incorporation into the silica of diatom cell walls, Arch. Mikrobiol. 92, 11 (1973); and F. Azam and B. E. Volcani, Role of silicon in diatom metabolism. VI. Active transport of germanic acid in the heterotrophic diatom Nitzschia alba, Arch. Mikrobiol. 101, 1 (1974). A later TEM study showed that concentrations of 0.07 mM Ge(OH)₄ and 0.7 mM Si(OH)₄ in the culture medium (10:1 ratio of Si to Ge) induced aberrations in N. alba frustule morphology. M. L. Chaippino et al., Effect of Germanic acid on developing cell walls of diatoms, Protoplasma 93, 191 (1977).

Recently, researchers at Georgia Institute of Technology have published a method for making particular zinc silicate-coated microparticles by chemical modification of the surfaces of diatom microshells. Microshells were exposed to a zinc acetate-bearing solution and fired at 700° C. ZnO nanoparticles were allowed to react with the underlying SiO₂ micro-shells at 1,050° C. to generate Zn₂SiO₄ coatings that conformed to the microshell surfaces. See, (1) U.S. Patent Application No. 20030099763, and (2) Ye Cai and Kenneth H. Sandhage, Zn₂SiO₄-coated microparticles with biologically-controlled 3D shapes, Phys. Stat. Sol., 202, No. 10 (2005).

Despite these prior efforts, the current inventors are unaware of a method useful for producing and isolating biogenic metal oxide materials that are made using biological systems, such as diatoms. For example, Sandhage et al.'s method produces silica or silicate materials. Sandhage et al. use diatom shells only as a “template” for materials processing. “Physical vapor deposition” subsequently was used by Sandhage et aL to coat the diatom shell with a thin coat of magnesium (Mg). This material was then heated sufficiently to allow magnesium to displace silicon (Si) to form a composite. All of these steps are done under vacuum at high temperature using “top down” processing techniques.

### SUMMARY

Disclosed embodiments of the present invention concern biological processes useful for making biogenic oxide materials that address shortcomings associated with, and provide a viable alternative to, chemical processes currently used to make metal oxides. One disclosed embodiment for making metal oxides, semimetal oxides, mixed metal oxides, mixed semimetal oxides, and/or mixed metal-semimetal oxides (collectively referred to as metal oxides or oxide materials, unless the context indicates otherwise) comprises selecting a substrate for making the desired oxide, such as a photoluminescent Si—Ge oxide; contacting a biological system with the substrate material for a period of time effective for the system to assimilate the substrate material; and optionally, but most typically, isolating the desired oxide material from the biological system, such as by oxidizing organic biomass material. The substrate typically is an aqueous soluble source of an oxide material, particularly a Group IIIa, Group IVa and/or Group IVb metal oxide, such as a titanium oxide, silicon oxide, gallium oxide, germanium oxide, tin oxide, lead oxide, and combinations thereof. The substrate also can be a material that provides an aqueous, soluble source of a metal oxide, such as through hydrolysis of a metal halide, a metal hydride, a metal alkoxide, or combinations thereof.

The method may involve cultivating the biological system in the presence of a soluble source of silicon, such as silicic acid, to substantial silicon starvation prior to contacting the biological system with the substrate. A soluble source of silicon then may be coadministered with the substrate. For embodiments employing germanium, the soluble source of germanium typically was germanic acid.

Certain disclosed embodiments further comprise treating biomass produced after cultivation with an oxidizing agent. A person of ordinary skill in the art will appreciate that the oxidizing agent can be any suitable oxidizing agent including, but not limited to, oxygen, hydrogen peroxide, a permanganate, and combinations thereof.

Certain disclosed embodiments also further comprise post processing a primary or secondary material. These embodiments can include combining the primary or secondary material with at least one metal, metal oxide, mixed metal oxide, mixed metal-semimetal oxide, or combinations thereof. For example, combining may comprise chemical bath deposition and/or vapor phase deposition.

Disclosed embodiments of the present method can be used to produce luminescent materials having different emission characteristics. Disclosed embodiments also can include producing nanostructured metal oxides, and/or metal oxides having a biogenic microstructure.

Composite phosphor materials can be made by the method. Examples of such phosphors include ZnS:Mn/SiO₂:Ge, ZnS:Tb/SiO₂:Ge, ZnS:Sm,Cl/SiO₂:Ge, ZnS:TbOF/SiO₂:Ge, ZnS:Tm,F/SiO₂:Ge, CaS:Eu/SiO₂:Ge, CaS:Ce,Cl/SiO₂:Ge, CaSSe:Eu/SiO₂:Ge, SrS:Ce,F/SiO₂:Ge, SrS:Ce,Mn,Cl/SiO₂:Ge, SrS:Ce/SiO₂:Ge, SrS:Ce/SiO₂:Ge, Eu/SiO₂:Ge, ZnO:Ga/SiO₂:Ge, ZnO:Al/SiO₂:Ge, ZnO:Cu/SiO₂:Ge, ZnSe:Cu/SiO₂:Ge, ZnSe:Cu,Cl/SiO₂:Ge, ZnSe:Mn/SiO₂:Ge, ZnTe:Mn/SiO₂:Ge, ZnTe:Eu/SiO₂:Ge, CdS:Ag/SiO₂:Ge, CdS:Te/SiO₂:Ge, CdS:In/SiO₂:Ge, CdS:Te/SiO₂:Ge, CdSe:Cl/SiO₂:Ge, CdSe:Ag/SiO₂:Ge, CdTe:Ag/SiO₂:Ge, CdTe:Cy/SiO₂:Ge, CdO:Mn/SiO₂:Ge, CdO:Tm/SiO₂:Ge, CaS:Ce/SiO₂:Ge, CaS:Eu/SiO₂:Ge, CaS:Pb/SiO₂:Ge, CaS:Sm/SiO₂:Ge, CaSe:Ce/SiO₂:Ge, CaSe:Mn/SiO₂:Ge, CaSe:Tm/SiO₂:Ge, SrS:Ce/SiO₂:Ge, SrS:Eu/SiO₂:Ge, SrS:Mn/SiO₂:Ge, SrSe:Ba/SiO₂:Ge, SrSe:Ce/SiO₂:Ge, BaS:Au/SiO₂:Ge, BaS:Ce/SiO₂:Ge, BaS:Mn/SiO₂:Ge, BaSe:Eu/SiO₂:Ge, BaSe:Mn/SiO₂:Ge, LaPO₄:Eu/SiO₂:Ge, LaPO₄:Ce/SiO₂:Ge, CePO₄:Eu/SiO₂:Ge, CePO₄:Ce/SiO₂:Ge, GdPO₄:Tb/SiO₂:Ge, GdPO₄:Ce/SiO₂:Ge, YPO₄:Eu/SiO₂:Ge, YPO₄:Mn/SiO₂:Ge, YPO₄:V/SiO₂:Ge, ScBO₃:Tb/SiO₂:Ge, ScBO₃:Eu/SiO₂:Ge, ScBO₃:Ce/SiO₂:Ge, YBO₃:Eu/SiO₂:Ge, YBO₃:Ce/SiO₂:Ge, LaBO₃:Eu/SiO₂:Ge, LaBO₃:Mn/SiO₂:Ge, CeBO₃:Eu/SiO₂:Ge, CeBO₃:Tb/SiO₂:Ge, PbMoO₄:Eu/SiO₂:Ge, PbMoO₄:Mn/SiO₂:Ge, CaMoO₄:Eu/SiO₂:Ge, CaMoO₄:Ce/SiO₂:Ge, CdMoO₄:Eu/SiO₂:Ge, CdMoO₄:Mn/SiO₂:Ge, ZnMoO₄:Eu/SiO₂:Ge, ZnMoO₄:Ce/SiO₂:Ge, CdMoO₄:Mn/SiO₂:Ge, BaMoO₄:Mn/SiO₂:Ge, BaMoO₄:Eu/SiO₂:Ge, BaMoO₄:Tb/SiO₂:Ge, Gd₂(MoO₄):Mn/SiO₂:Ge, Gd₂(MoO₄):Tb/SiO₂:Ge, YVO₄:Eu/SiO₂:Ge, YVO₄:Dy/SiO₂:Ge, YPO₄:Eu/SiO₂:Ge, YPO₄:Mn/SiO₂:Ge, YPO₄:Mn,Th/SiO₂:Ge, CaWO₄:Sm/SiO₂:Ge, CaWO₄:Pb/SiO₂:Ge, PbWO₄:Tb/SiO₂:Ge, PbWO₄:Ce/SiO₂:Ge, and any and all combinations thereof. Specific examples of silicate-based phosphor materials include CaSiO₃:Ce, CaSiO₃:Eu, CaSiO₃:Pb, CaSiO₃:Ti, CaSiO₃:Pb,Mn, Be₂SiO ₄:Mn, Mg₂SiO₄:Mn, Zn₂SiO₄:Mn, Zn₂SiO₄:Mn,P, Zn₂SiO₄:Mn,As, Zn₂SiO₄:Ti, (Zn+Be)₂SiO₄:Mn, Sr₂SiO₄:Eu, SrBaSiO₄:Eu, Ba₂SiO₄:Eu, Ba₂SiO₄:Ce,Li,Mn, BaSi₂₅:Eu, BaSi₂₅:Pb, Y₂SiO₅:Ce, CaMgSi₂O₆:Eu, CaMgSi₂O₆:Eu,Mn, Ca₂MgSi₂O₇:Eu, Ca₂MgSi₂O₇, Ca₂MgSi₂O₇:Eu,Mn, Sr₂MgSi₂O₇:Eu, Ba₂MgSi₂O₇:Eu, BaMg₂Si₂O₇:Eu, BaSrMgSi₂O₇:Eu, Ba₂Li₂Si₂O₇:Eu, Ba₂MgSi₂O₇:Sn, Ba₂MgSi₂O₇:Sn,Mn, MgSrBa₂Si₂O₇:Eu, MgBa₃Si₂O₈:Eu, MgSr₃Si₂O₈:Eu,Mn, Sr₃MgSi₂O₈:Eu, Ca₅B₂SiO10:Eu, Ca₃Al₂Si₃O₁₂:Eu, LiCeBa₄Si₄O₁₄:Mn, LiCeSrBa₃Si₄O₁₄:Mn, and any and all combinations thereof.

The biological system can be an organism, most typically a living organism, but also can be isolated materials or compositions, such as proteins, that assimilate substrate materials. The Microalgae provide one example of a class of living organisms useful for making oxide materials, such as the Bacillariophyceae. Working embodiments used diatoms, including marine Pennate and/or Centric diatoms, such as Nitzschia, Pinnularia, and/or Cyclotella diatoms. Specific examples of diatoms include species within the genus Nitzschia, such as Nitzschia acicularis, Nitzschia alba, Nitzschia alexandrina, Nitzschia amphibian, Nitzschia apiculata, Nitzschia closterium, Nitzschia communis, Nitzschia compressa, Nitzschia dissipata, Nitzschia distans, Nitzschiafiliformis, Nitzschia fonticola, Nitzschia frustulum, Nitzschia fusiformis, Nitzschia inconspicua, Nitzschia laevis, Nitzschia lecointei, Nitzschia linearis, Nitzschia longissima, Nitzschia navis-varingica, Nitzschia palea, Nitzschia paleacea, Nitzschia paleaformis, Nitzschia pellucida, Nitzschia pusilla, Nitzschia sigma, Nitzschia supralitorea, Nitzschia thermalis, Nitzschia thermaloides, Nitzschia vitrea; species within the genus Pinnularia, including Pinnularia quadratarea and Pinnularia rupestri; species within the genus Cyclotella; and combinations thereof.

For particular working embodiments of the disclosed method that were used to make a Si—Ge oxide photoluminescent material, a Nitzschia or Pinnularia diatom was cultivated to substantial silicon starvation in the presence of silicic acid to produce a silicon-starved diatom. The silicon-starved diatom was cultivated for at least one cell division, and typically for a period of greater than 24 hours, in the presence of effective concentrations of germanic and silicic acid to produce a biomass. The biomass was subjected to an oxidation step, typically a chemical oxidation step, such as chemical oxidation with hydrogen peroxide, to remove organic materials. This allowed isolation of an amorphous, Si—Ge oxide photoluminescent material. Both cultivation and chemical oxidation were conducted at relatively low temperatures of less than 100° C.

The method also can involve post processing a primary or secondary oxide material that is produced biologically. A primary and/or secondary oxide material, or materials, produced by an initial biological process subsequently can be combined or reacted with another material to modify attributes of the primary or secondary material as desired. For example, a first photoluminescent material might be physically combined with a second, biogenically produced photoluminescent material. As a second example, a primary and/or secondary oxide material can be reacted or intimately combined with another material, such as by chemical bath deposition, vapor phase deposition and/or a SILAR process. Post processing has been used to produce luminescent materials having different emission characteristics than the primary or secondary biogenic material.

Biologically produced metal oxides can be used to make devices that utilize the metal oxide or oxides, or compound(s) made from the metal oxide, such as optoelectronic devices. The method involves making a primary or secondary oxide material biologically, such as by using a diatom cultivation-step; optionally, but most typically, removing the organic material, such as by a chemical oxidation step to isolate the biogenic oxide material; optionally post processing the primary or secondary metal oxide; and making an optoelectronic device comprising the oxide material(s). Examples of such devices include memory devices, data storage devices, capacitors, detectors, sensors, lasers, emitters, light-emitting diodes, optical computing architectures, photonics, imaging devices and photovoltaics.

An imaging method comprising using a biologically produced oxide material, particularly nanoparticles thereof, also is described. For example, a method for imaging tissue of a subject comprises making, a primary or secondary oxide material, generally as a nanoparticle; optionally, but most typically, isolating the nanoparticles from the biomass by chemical oxidation and particle separation; optionally post processing the primary or secondary oxide material; administering the oxide material to a subject; and imaging the subject by luminescence.

A method for administering a composition comprising an active agent, such as a therapeutic or diagnostic, and a biologically produced oxide material also is described. The biogenic oxide material, typically nanoclusters thereof, can be physically or chemically combined with at least one active agent. Alternatively, the active agent composition may be formed by imbedding active agent within a defined microstructure, such as diatom frustule apertures. A person of ordinary skill in the art will recognize that such a first therapeutic composition can be combined with at least a second therapeutic composition. Also, other materials commonly used to make therapeutic compositions, such as excipients, can be used to make therapeutic compositions as well. The therapeutic composition is administered to a subject. Therapeutic release may be induced or facilitated by initiating energy absorption by the composition, such as by using light absorption from a light source external to the subject.

Examples of classes of useful active agents include nucleic acids, proteins, naturally occurring organic compounds, synthetic and semi-synthetic compounds, and combinations thereof. More particularly, the diagnostic or therapeutic agent may be an AIDS adjunct agent, alcohol abuse preparation, Alzheimer's disease management agent, amyotrophic lateral sclerosis therapeutic agent, analgesic, anesthetic, antacid, antiarythmic, antibiotic, anticonvulsant, antidepressant, antidiabetic agent, antiemetic, antidote, antifibrosis therapeutic agent, antifungal, antihistamine, antihypertensive, anti-infective agent, antimicrobial, antineoplastic, antipsychotic, antiparkinsonian agent, antirheumatic agent, appetite stimulant, appetite suppressant, biological response modifier, biological, blood modifier, bone metabolism regulator, cardioprotective agent, cardiovascular agent, central nervous system stimulant, cholinesterase inhibitor, contraceptive, cystic fibrosis management agent, deodorant, diagnostic, dietary supplement, diuretic, dopamine receptor agonist, endometriosis management agent, enzyme, erectile dysfunction therapeutic, fatty acid, gastrointestinal agent, Gaucher's disease management agent, gout preparation, homeopathic remedy, hormone, hypercalcemia management agent, hypnotic, hypocalcemia management agent, immunomodulator, immunosuppressive, ion exchange resin, levocamitine deficiency management agent, mast cell stabilizer, migraine preparation, motion sickness product, multiple sclerosis management agent, muscle relaxant, narcotic detoxification agent, narcotic, nucleoside analog, non-steroidal anti-inflammatory drug, obesity management agent, osteoporosis preparation, oxytocic, parasympatholytic, parasympathomimetic, phosphate binder, porphyria agent, psychotherapeutic agent, radio-opaque agent, psychotropic, sclerosing agent, sedative, sickle cell anemia management agent, smoking cessation aid, steroid, stimulant, sympatholytic, sympathomimetic, Tourette's syndrome agent, tremor, preparation, urinary tract agent, vaginal preparation, vasodilator, vertigo agent, weight loss agent, Wilson's disease management agent, and mixtures thereof. Specific examples of such active agents include abacavir sulfate, abacavir sulfate/lamivudine/zidovudine, acetazolamide, acyclovir, albendazole, albuterol, aldactone, allopurinol BP, amoxicillin, amoxicillin/clavulanate potassium, amprenavir, atovaquone, atovaquone and proguanil hydrochloride, atracurium besylate, beclomethasone dipropionate, berlactone betamethasone valerate, bupropion hydrochloride, bupropion hydrochloride SR, captopril, carvedilol, caspofingin acetate, cefazolin, ceftazidime, cefuroxime (no sulfate), chlorambucil, chlorpromazine, cimetidine, cimetidine hydrochloride, cisatracurium besilate, clobetasol propionate, co-trimoxazole, colfosceril palmitate, dextroamphetamie sulfate, digoxin, enalapril maleate, epoprostenol, esomepraxole magnesium, fexofenadine, fluticasone propionate, furosemide, gancyclovir, hydrochlorothiazide/triamterene, lamivudine, lamotrigine, lithium carbonate, losartan potassium, melphalan, mercaptopurine, mesalazine, metformin, methyldopa, minocycline, mupirocin calcium cream, nabumetone, naratriptan, omeprazole, ondansetron hydrochloride, orli stat (or a pharmaceutically acceptable salt thereof), ovine, oxiconazole nitrate, paroxetine hydrochloride, prochlorperazine, procyclidine hydrochloride, pyrimethamine, ranitidine bismuth citrate, ranitidine hydrochloride, rofecoxib, ropinirole hydrochloride, rosiglitazone maleate, salmeterol xinafoate, salmeterol, selegiline, fluticasone propionate, sterile ticarcillin disodium/clavulanate potassium, simvastatin, spironolactone, succinylcholine chloride, sumatriptan, thioguanine, tirofiban HCI, topotecan hydrochloride, tranylcypromine sulfate, trifluoperazine hydrochloride, valacyclovir hydrochloride, vinorelbine, zanamivir, zidovudine, zidovudine, lamivudine, and combinations thereof.

The foregoing and other features and advantages of the disclosed embodiments of the present invention will become more apparent from the following detailed description, which proceeds with reference to the accompanying figures.

### BRIEF DESCRIPTION OF THE DRAWINGS

The patent or application file contains at least one drawing executed in color. Copies of this patent or patent application publication with color drawings(s) will be provided by the Office upon request and payment of the necessary fee.

FIG. 1 is a graph of cell density (#cells/ml) versus cultivation time (hours) illustrating cultivation to substantial silicon starvation during stage 1 of a working embodiment of a biological production (cultivation) process.

FIG. 2 is a graph of germanium concentration (μmoles Ge/liter) versus cultivation time (hours) illustrating co-addition of soluble silicon and germanium to a diatom culture suspension during stage 2 of a working embodiment of a biological production process.

FIG. 3 is a graph of cell density (#cells/ml) versus cultivation time (hours) for a diatom for one cell division to allow production of a new frustule during stage 2 of a working embodiment of a biological production process.

FIG. 4 is a graph of the measured germanium and silicon concentration (μmol Ge/g dry cell mass, mmol Si/g dry cell mass) versus cultivation time (hours) illustrating co-addition of soluble silicon and germanium to a diatom-during stage 2 of a working embodiment of a biological production process.

FIG. 5 is a scanning electron microscopy (SEM) image of an intact frustule of Nitzschia frustulum illustrating structural order in the sub-micron range (scale bar is 1.0 micron).

FIG. 6 is a transmission electron microscopy (TEM) image of an intact frustule of Pinnularia sp. illustrating structural order in the sub-micron range (scale bar is 2.0 microns).

FIG. 7 is a TEM image of an intact frustule of Pinnularia sp. illustrating structural order in the nanoscale range (scale bar is 20 nanometers).

FIG. 8 is a TEM image of an aqueous-hydrogen-peroxide-treated, Nitzschia frustulum frustule having 0.6 wt % germanium in the amorphous white powder.

FIG. 9 is aTEM image of an aqueous-hydrogen-peroxide-treated, Nitzschia frustulum frustule and associated Si—Ge oxide nanoparticles containing 2.0 wt % germanium in the amorphous white powder, where the scale bar is 1.0 μm.

FIG. 10 presents photoluminescence (PL) spectra of a Nitzschia frustulum diatom frustule containing 0.25 wt % Ge processed by aqueous hydrogen peroxide treatment of the cell biomass alone as compared to sequential aqueous hydrogen peroxide treatment and thermal annealing treatment of the cell biomass. The PL spectra were obtained at room temperature with a 337 nm nitrogen laser excitation source at 2.0 mW average power input and integration time of 2,000 msec.

FIG. 11 presents photoluminescence (PL) spectra of a Nitzschia frustulum diatom frustule containing 0.50 wt % Ge processed by aqueous hydrogen peroxide treatment of the cell biomass as compared to direct thermal annealing treatment of the cell biomass. The PL spectra were obtained at room temperature with a 337 nm nitrogen laser excitation source at 2.0 mW average input and integration time of 2,000 msec.

FIG. 12 is a photomicrograph illustrating blue photoluminescence of biogenic nanostructured Si—Ge oxide powder under UV light.

FIG. 13 is a graph of silicon concentration (mg Si/liter) and cell density (10⁶ cells/ml) versus cultivation time (hours) showing growth and soluble Si consumption from the liquid medium as N. frustulum diatoms achieve silicon starvation.

FIG. 14 is a graph of silicon concentration (mg Si/liter) and cell density (10⁶ cells/ml) versus time after germanium addition (hours) illustrating soluble Ge consumption after pulse addition of a mixture of Ge and Si to Si-starved diatom cell suspension culture with initial concentration of 1.60 mg of Ge/liter (0.022 mM), 7.31 mg of Si/L (0.25 mM), and a Ge:Si ratio of 11.8:1.

FIG. 15 is a graph of germanium uptake (mg Ge/g dry cells) versus time after addition of germanium (hours) comparing germanium uptake in N. frustulum cell mass following pulse addition of soluble germanium alone (7.45 mg of Ge/L) or a mixture of soluble Ge and Si (1.6 mg of Ge/L, 7.3 mg of Si/L) to a Si-starved diatom cell suspension culture illustrating short-term uptake during first 5 hours.

FIG. 16 is a graph of germanium uptake (mg Ge/g dry cells) versus time after addition of germanium (hours) comparing germanium uptake in N. frustulum cell mass following pulse addition of soluble germanium alone (7.45 mg of GeAL) or a mixture of soluble Ge and Si (1.6 mg of Ge/L, 7.3 mg of Si/L) to Si-starved diatom cell suspension culture illustrating long-term uptake.

FIG. 17 is an SEM of N. frustulum frustules showing ribbed and honeycomb microstructures.

FIG. 18 is a high magnification SEM of thermally annealed N. frustulum diatom illustrating that the diatom shell remained intact after thermal annealing.

FIG. 19 is a Brightfield TEM of nanoclustured fragments fractured from thermally annealed N. frustulum cell mass after 123 hours of pulse addition of soluble germanium alone (7.45 mg Ge/L) to a Si-starved diatom cell suspension culture illustrating imbedded nanoclusters.

FIG. 20 is a TEM-EDS line scan along the line illustrated in FIG. 19 through a nanocluster of Ge and Si.

FIG. 21 is a TEM-EDS line scan along the line illustrated in FIG. 19 through a nanocluster of Ge and Ca.

FIG. 22 is a graph of weight composition versus analysis position illustrating quantitative elemental analysis of Ge and Si atoms by TEM-EDS along the line illustrated in FIG. 19.

FIG. 23 is a graph of weight composition versus analysis position illustrating quantitative elemental analysis of Ca, Mg and O atoms by TEM-EDS along the line illustrated in FIG. 19.

FIG. 24 is a full scan (400 to 4,000 cm⁻¹) FT-IR spectra of thermally annealed N. frustulum cell mass following pulse addition of soluble Ge (7.45 mg of Ge/L) to the Si-starved diatom cell suspension.

FIG. 25 is a partial scan (400to 1,200 cm⁻¹) FT-IR spectra of thermally annealed N. frustulum cell mass following pulse addition of soluble Ge (7.45 mg of Ge/L) to the Si-starved diatom cell suspension.

FIG. 26 is an FT-IR spectra of standards including crystalline GeO₂, crystalline CaCO₃ and thermally annealed (800° C., 6 hours, air) crystalline mixture of 10:1 M CaCO₃ and GeO₂.

FIG. 27 is an XRD analysis of thermally annealed N. frustulum cell mass, obtained 123 hours after pulse addition of a mixture of soluble Ge and Si (1.6 mg of Ge/L; 7.3 mg of Si/L) to a Si-starved diatom cell suspension culture for comparison to a standard of crystalline Ca₃GeO₅.

FIG. 28 is an XRD analysis of thermally annealed N. frustulum cell mass, obtained 123 hours after pulse addition of a mixture of soluble Ge and Si (1.6 mg of Ge/L, 7.3 mg of Si/L) to a Si-starved diatom cell suspension culture for comparison to a standard of crystalline CaSiO₃.

FIG. 29 is an XRD analysis of thermally annealed N. frustulum cell mass, obtained 123 hours after pulse addition of a mixture of soluble Ge and Si (1.6 mg of Ge/L, 7.3 mg of Si/L) to a Si-starved diatom cell suspension culture for comparison to thermally annealed N. frustulum cell mass just before pulse addition of soluble Ge and Si.

FIG. 30 is a schematic drawing of an electroluminescent device using a thin layer of diatom frustules as the emission source.

FIG. 31 is schematic drawing illustrating a representative addressable display based on luminescence.

FIG. 32 is a digital image of ZnS:Mn²⁺/SiO₂:Ge made using silica isolated from a Nitzschia diatom, hydrogen peroxide oxidation of organic material and SILAR post processing.

FIG. 33 is a digital image of ZnS:Mn²⁺/SiO₂:Ge made using a Pinnularia diatom, hydrogen peroxide oxidation of organic material and SILAR post processing.

FIG. 34 is a digital image of ZnSiO₄:Mn²⁺ made using silica isolated from a Nitzschia diatom, hydrogen peroxide oxidation of organic material and CBD post processing.

FIG. 35 is a digital image of ZnSiO₄:Mn²⁺ made using silica isolated from a Pinnularia diatom, hydrogen peroxide oxidation of organic material and CBD post processing.

FIG. 36 is a photoluminescence spectrum of ZnSiO₄:Mn²⁺ made using silica isolated from a Pinnularia diatom, hydrogen peroxide oxidation of organic material and CBD post processing.

FIG. 37 is a digital image of Sr₂Si₂O₇:Eu²⁺ made using silica isolated from a Pinnularia diatom, hydrogen peroxide oxidation of organic material and CBD post processing.

FIG. 38 is a photoluminescence spectrum of Sr₂Si₂O₇:Eu²⁺ made using silica isolated from a Pinnularia diatom, hydrogen peroxide oxidation of organic material and CBD post processing.

FIG. 39 is a digital image of Sr₂MgSi₂O₇:Eu²⁺ made using silica isolated from a Pinnularia diatom, hydrogen peroxide oxidation of organic material and CBD post processing.

FIG. 40 is a digital image of SrBaSiO₄:Eu⁺ made using silica isolated from a Pinnularia diatom, hydrogen peroxide oxidation or organic material and CBD post processing.

FIG. 41 is a schematic of bubble-column photobioreactor.

FIG. 42 is a graph of cell number density (X_{N}) and soluble Si concentration in culture medium (C_{Si}) versus time for two-stage photobioreactor cultivation of the marine diatom Pinnularia sp. at Stage II initial concentrations of 0.0 μmol Ge/L and 0.53 mmol Si/L.

FIG. 43 is a graph of cell number density (X_{N}) and soluble Si concentration in culture medium (C_{Si}) versus time for two-stage photobioreactor cultivation of the marine diatom Pinnularia sp. at Stage II initial concentrations of 38 μmol Ge/L and 0.53 mmol Si/L.

FIG. 44 is a graph of soluble Si and Ge concentration in the culture medium during Stage II of cultivation for experiments conducted at the Stage II initial concentration of 17 μmol Ge/L and 0.55 mmol Si/L.

FIG. 45 is a graph of soluble Si and Ge concentration in the culture medium during Stage II of cultivation for experiments conducted at the Stage II initial concentration of 38 μmol Ge/L and 0.53 mmol Si/L.

FIG. 46 is a graph showing the effect of Stage II initial Ge concentration on the Si content in cell biomass (Y_{Si}, mmol Si/g DW) at Stage II cultivation time of 56 hours.

FIG. 47 is a graph showing the effect of Stage II initial Ge concentration on the Ge content in cell biomass (Y_{Ge}, μmol Ge/g DW) at Stage II cultivation times of 12 and 56 hours.

FIG. 48 is a graph showing the effect of Stage II initial Ge concentration on the Ge content in hydrogen peroxide treated silica frustule (wt % Ge in silica) at Stage II cultivation times of 12 and 56 hours.

FIG. 49 is an SEM image of intact silica frustule of Pinnularia sp. obtained at the end of Stage I cultivation showing the side (girdle band) view.

FIG. 50 is an SEM image of intact silica frustule of Pinnularia sp. obtained at the end of Stage I cultivation showing the pore array.

FIGS. 51-56 provide TEM images of intact silica frustule of Pinnularia sp. obtained after 56 hours of Stage II cultivation. (a)-(c) micron, submicron, and nanostructure at Stage II initial concentrations of 0 μmol Ge/L Ge (control) and 0.53 mmol Si/L; (d)-(f) micron, submicron, and nanostructure at Stage II initial concentrations of 38 μmol Ge/L and 0.53 mmol Si/L.

FIGS. 57-68 show STEM-EDS analyses of intact silica frustule of Pinnularia sp. obtained after 56 hours of Stage II cultivation at initial Ge concentration of 38 μmol/L and initial Si concentration of 0.53 mmol/L. STEM image and Si+Ge line scans: (FIGS. 56 and 57) across entire frustule; (FIGS. 58 and 59) across a series of ribs (costae); (FIGS. 60 and 61) between two pores; (FIGS. 62 and 63) through nanostructure of single areolae; (FIGS. 64 and 65) through a region containing Ge-rich nanoparticles covering a pore; (FIGS. 66 and 67) along the length of a putative girdle band partially separated from its valve.

FIG. 69 shows photoluminescence (PL) spectra of Pinnularia sp. frustule powder obtained from hydrogen peroxide treatment of cell mass at Stage II cultivation times of 0, 12, and 56 hours at Stage II initial concentrations of 0 μmol Ge/L (control) and 0.53 mmol Si/L, yielding a frustule material which possessed 0.0 wt % germanium after 56 hours.

FIG. 70 shows photoluminescence (PL) spectra of Pinnularia sp. frustule powder obtained from hydrogen peroxide treatment of cell mass at Stage II cultivation times of 0, 12, and 56 hours at Stage II initial concentrations of 38 μmol Ge/L and 0.53 mmol Si/L, yielding a frustule material which possessed 0.96% germanium after 56 hours.

FIG. 71 compares the peak wavelength of the photoluminescence (PL) spectra of Pinnularia sp. frustule powder obtained from hydrogen peroxide treatment of cell mass at Stage II cultivation times of 0, 12, and 56 hours.

FIG. 72 is a comparison of the electroluminescence spectrum and photoluminescence spectrum for Pinnularia sp. frustules containing 0.96 wt % germanium.

FIG. 73 is a TEM micrograph showing nanocrystalline Ge imbedded into the diatom silica frustule.

FIG. 74 is an XRD spectrum of the diatom silica frustule showing the presence of crystalline Ge.

FIG. 75 is a photoluminescence (PL) spectrum of nanocrystalline Ge nanoparticles imbedded in the diatom biosilica at an excitation wavelength of 254 nm for two different loadings of Ge in the diatom biosilica. For comparison, the PL spectrum of the Delron sample holder itself with no sample present under UV lamp excitation at 254 nm is also provided.

FIG. 76 is an SEM image of the Pinnularia sp. frustule before chemical bath deposition.

FIGS. 77A-B are SEM images of the Pinnularia sp. frustule after conversion to zinc silicate doped with manganese.

FIG. 78 is a TEM image of type I nanostructure.

FIG. 79 is a TEM lattice image of Zn₂SiO₄:Mn nanocrystals.

FIG. 80 is a TEM image of type II nanostructure.

FIG. 81 is an EDS line scan for zinc through a representative type II nanostructure.

FIG. 82 is a powder X-ray diffraction and electron diffraction patterns of Zn₂SiO₄:Mn obtained from the Pinnularia sp. frustule and the corresponding JCPDS-85-0453 reference patterns.

FIG. 83 is the photoluminescent spectrum of Zn₂SiO₄:Mn coated Pinnularia sp. frustule (Inset: green luminescent emission of bulk powder).

FIG. 84 is a light microscope image of a Zn₂SiO₄:Mn coated Pinnularia sp. frustule showing the intact features of the frustule under the white light illumination.

FIG. 85 is a light microscope image of a Zn₂SiO₄:Mn coated Pinnularia sp. frustule showing the intact features of the frustule under UV light illumination.

FIG. 86 is a reaction scheme for amine functionalization of diatom biosilica with the reagent 3-APS (3-aminopropyl-trimethoxysilane).

FIG. 87 is a fluorescence microscope image showing amine-functionalized frustule of Pinnularia sp. as visualized by Fluorsecamine staining.

FIG. 88 is a fluorescence microscope image showing amine-functionalized frustule of Cyclotella sp. as visualized by Fluorsecamine staining.

### DETAILED DESCRIPTION

### I. Introduction

The disclosed embodiments generally involve selecting appropriate substrate materials that are useful for forming desired materials, typically oxide materials, most typically metal oxides, such as Groups IIIa, IVa and/or IVb metal oxides. Selected substrates are provided to a biological system for synthesis of desired compounds. One disclosed working embodiment concerns a bioreactor cultivation process that promotes the uptake of desired substrates by diatoms. A biological system, such as living cells and their proteins, do all of the work at ambient-conditions using simple ingredients with little or no waste. Consequently, no exotic and expensive equipment, extreme temperatures and/or pressures, or toxic chemicals are required. Furthermore, biological systems use “bottom up,” processes, meaning that nanoscale elements are chemically synthesized at the molecular level and then “self assembled” into defined microscale patterns in ways that cannot presently be duplicated by top-down processes.

Metal oxide materials synthesized by the biological system are isolated from the biomass after substrate assimilation. Materials made by particular embodiments of the method can be made to include microstructures that facilitate optoelectronic and/or other device applications.

Once desired photoluminescent materials have been isolated from the biological system, they may be further processed, if necessary, such as by techniques that facilitate their use, including drying, purification and/or other physical treatments. Moreover, subsequent biological, physical and/or chemical processes also can be applied to material isolated from a first biological system to make materials that have properties other than those of materials initially produced by the biological system. Examples of such post processing techniques include chemical deposition processes, such as chemical bath deposition, vapor phase deposition, and/or successive ionic layer adsorption and reaction (SILAR).

As an alternative, biological systems can produce primary materials that are normally produced by the organism or organisms during a life cycle. For example, again with reference to the diatoms, the frustule of such organisms includes nanostructured silicon oxides that do have useful properties without physical or chemical modification, including photoluminescent capabilities. Frustules formed from nanostructured silicon oxides possess defined microstructures that also have useful properties, including photonic and drug delivery capabilities. These primary materials can be isolated, and then perhaps further treated, either physically and/or chemically, where necessary or desirable to provide additional benefits. Again by way of example, such materials can be chemically modified by combination or reaction with metals, semimetals, metal oxides, mixed metal oxides, mixed semimetal oxides, and/or mixed semimetal-metal oxides, to provide photoluminescent enhancement or to modify the absorption or emission characteristics.

### II. Definitions

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

“Active agent” means any therapeutic or diagnostic agent now known or hereafter discovered that can be formulated as described herein. Examples of therapeutics, without limitation, are listed in U.S. Pat. No. 4,649,043, which is incorporated herein by reference. Additional examples are listed in the American Druggist, p. 21-24 (February, 1995).

“Administration” to a subject can be by any known means including, but not limited to, orally, vaginally, rectally, nasally, or in the oral cavity.

Unless context indicates otherwise, “ambient temperature” is any temperature between about 0° C. and about 100° C., more typically from about 5° C. to about 50° C. and even more typically from about 10° C. and about 40° C.

A “biological system” refers to an organism, such as a diatom, most typically a living organism, or to the components derived from an organism useful for making oxide materials as described herein, e.g. a mixture comprising cellular components, such as proteins, that synthesize the desired metal oxide materials. “Diagnostic” means, without limitation, a material useful for testing for the presence or absence of a material or disease, and/or a material that enhances tissue or cavity imaging.

An “effective amount” is an amount of a diagnostic or therapeutic agent that is useful for producing a desired effect.

“Metal oxide” refers to a particular material, or can refer collectively to a group of materials, such as a group that includes metal oxides, mixed metal oxides, semimetal oxides, mixed semimetal oxides, mixed metal-semimetal oxides, metal hydroxides, mixed metal hydroxides, semimetal hydroxides, mixed semimetal hydroxides, mixed metal-semimetal hydroxides, and any and all possible combinations thereof.

A “microstructure” is any feature size of greater than about 0.10 microns and less than about 1000 microns. A “microstructure” typically arises as a result of assembling nanostructured compositions made by the biological system. A microstructure might be a repeating unit present in a diatom frustule, such as frustule apertures. These repeating structures can be maintained during the production process of disclosed embodiments to facilitate certain applications. For example, frustule apertures can act as a waveguide to control propagation of an electromagnetic wave so that the wave is forced to follow a path defined by the physical structure. Changes in the frustule microstructure, such as variations in distances between frustule apertures or different aperture diameters, can be used to modify waveguide properties. Solely by way of example, frustule apertures produced by N. frustulum diatoms have diameters of about 100 nanometers, and the distance between frustule apertures is from about 50 to about 100 nanometers. Pinnularia apertures have diameters of about 200 nanometers and are separated by about 200 nanometers.

A “nanostructure” is any feature of a composition having a feature size of less than about 100 nanometers.

A “primary material” is a metal oxide or metal hydroxide material normally produced by the biological system(s), such as a silica nanocluster, in the absence of an added substrate material.

A “secondary material” is a metal oxide or metal hydroxide material produced by a biological system from a substrate(s), such as Si—Ge oxide nanocluster secondary materials that are produced using a germanium substrate.

“Semimetal” (also metalloid) means a class of elements intermediate in properties between metal and nonmetals. “Semimetal” includes, but is not limited to, boron, silicon, gallium, germanium, arsenic and tellurium. There is no clean distinction between metal and semimetal, and a particular element may be classified in more than one class.

A “substrate” or “substrate material” refers generally to a compound, or compounds, provided to a biological system for synthesis of a desired metal oxide or metal hydroxide, or a compound or compounds that form a metal oxide or metal hydroxide.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

### III. Substrate Materials

Certain disclosed embodiments of the present invention concern production of primary or secondary metal oxides. As such, suitable substrates for producing desired metal oxides are selected. Several functional criteria can be considered to guide selection of substrate materials. First, as would be apparent, the biological system must be able to assimilate the substrate, and subsequently synthesize either a primary or secondary material of interest. Materials that are substantially toxic to the organism at concentrations useful for making primary or secondary materials, or which cannot be utilized by appropriate cellular components, likely are not acceptable substrate materials.

For example, biological systems may use cell transport mechanisms to transport substrate materials into the cell. Useful substrate materials should be recognized by the cell transport mechanism sufficiently to provide an acceptable uptake rate and/or uptake amount. By way of example, and without limitation, the substrate transport mechanism for the genus Nitzschia recognizes substantially tetrahedrally shaped metal hydroxides soluble in water.

The pH of the cultivation media also can be a factor for two primary reasons, substrate speciation and physiologically acceptable pH values. Metal oxides may adopt different chemical species as pH varies. Species for diatom uptake preferably are protonated. The acceptable pH range is that which provides hydroxyl protonation, a pH range that typically is from about 6 to about 10. However, diatoms may be adversely affected if the pH is too much beyond the physiologically acceptable pH, which typically is from about 7.5 to about 10.

A second functional criterion is that the substrate material should produce a useful metal oxide material. The useful metal oxide can be a primary or a secondary material having useful properties without further processing, or can be subjected to further processing.

A third functional criterion is that the substrate material should produce a metal oxide material that is amorphous.

A fourth functional criterion is that the substrate material should produce a metal oxide material that is nanostructured.

A fifth functional criterion is that the substrate material should produce a secondary mixed metal oxide material that is nanostructured.

A six functional criterion is that the substrate material should be assimilated at ambient temperature, preferably within a temperature range of 5° C. to 40° C.

A seventh functional requirement is that the substrate materials could produce a mixed metal oxide material that possesses hierarchical structure, spanning the nanoscale (1 to 100 nm) and the micron scale (0.1 to 1000 microns).

Substrate materials for use in disclosed embodiments of the present invention typically are metal oxides, even more typically metal hydroxides, and most typically are aqueous, tetrahedral metal hydroxides. Materials that produce metal oxides, such as by hydrolysis, also are possible substrates, including metal halides, metal hydrides, metal alkoxides, metal silicates, such as sodium silicate, silicon oxide-based materials containing alkyl groups, such as lower alkyl groups, including methyl and ethyl (designated SiOC materials), etc., as long as such materials can be assimilated by the biological system. A currently preferred group of substrate materials is the Group IVb metals, which includes silicon, germanium, tin and lead. Group IVa metals also are useful, including titanium, zirconium, and hafnium. Furthermore, Group IIIa metals also may be useful, including gallium. As a result, a specific list of metal oxides useful for producing materials according to disclosed embodiments of the present invention include titanium oxides, zirconium oxides, hafnium oxides, silicon oxides, gallium oxides, germanium oxides, tin oxides, lead oxides, boron oxides, gallium oxides, indium oxides, and vanadium oxides, materials that produce such metal oxides, and all combinations thereof.

Working embodiments of the present invention have used an aqueous soluble source of germanium oxide, such as germanic acid [Ge(OH)₄] to exemplify substrate assimilation and metal oxide biosynthesis. The soluble source of germanic acid typically was co-administered, such as by pulse addition, with a soluble source of silicon, such as silicic acid. Germanium is of interest because silicon-germanium composite materials exhibit novel semiconducting and optoelectronic properties with microelectronic device applications. Rajeshwar, N. R. de Tacconi, and C. R. Chenthamarakshan, Semiconductor-based composite materials: Preparation, properties, and performance, Chem. Mater. 13, 2765 (2001).

### IV. Biological Systems

In nature, many marine and aquatic organisms assimilate inorganic materials from water to make ornate shell casings that possess defined nano- and microstructures. According to embodiments of the present invention, such biological systems allow for production of suitable materials without the concomitant production of toxic chemical waste that is generated by known chemical synthesis. Diatoms provide one example, without limitation, of an organism useful for producing metal oxides.

Diatoms generally are various microscopic, one-celled or colonial algae of the class Bacillariophyceae, and are a paradigm for controlled production of nanostructured silica. Diatoms possess a unique ability to actively take up soluble silicon from their surroundings and then biologically polymerize the intracellular silicon into silica nanospheres ranging from about 1 to about 100 nanometers; more typically from about 5 to about 50 nanometers, depending upon the species. Nanosphere diameters within a given diatom cell frustule are substantially monodisperse. Silica nanospheres then assemble into shells called “frustules” having an overall size that ranges from less than 5 microns to over 500 microns. Frustules possess defined microstructures, including periodic arrays of holes, ribs, etc., that are ordered at the microscale and the nanoscale. Microstructure is unique to a given diatom species. Nearly 100,000 species of diatoms are suspected to exist, and the frustules possess a myriad of shapes (round, oval, triangular, etc) at the microscale and ordered features at the nanoscale. These microstructures provide templates for fabricating a variety of microscale devices.

Diatoms possess subcellular machinery uniquely tailored for the fabrication of nanostructured metal oxides. Silica transporter proteins imbedded in the cell wall of the organism bring soluble silicon, such as Si(OH)₄ (silicic acid), into the cell. Within the cell, the silica deposition vesicle (SDV) is the “nanobioreactor” for biosilification. Following cytokinesis, the SDV forms on the interior of the plasmalemma. J. Pickett-Heaps, A. Schmid, and L. A. Edgar, The cell biology of diatom valve formation, Progress in Phycological Research, edited by F. E. Round and D. J. Chapman, Biopress Ltd., Bristol, (1990), pages 1-168. The SDV contains proteins called silaffins, which consist of a cationic polypeptide backbone post-translationally modified with long-chain oligo-N-methylpropylamines. N. Kröger et al., Self-assembly of highly phosphorylated silaffins and their function in biosilica morphogenesis, Science 298, 584 (2002). These silaffin proteins catalyze the condensation and precipitation of silicic acid to hard, monodisperse silica nanospheres, with the extent of precipitation controlled by a phase-partitioning process. The intricate microscale patterns created by nanosphere deposition also are species specific and are replicated upon cell division, suggesting molecular genetic control of the assembly process.

Diatoms for use in practicing disclosed embodiments of the present invention can be either heterotrophic or photosynthetic, and can be either freshwater aquatic or marine, and include both the Centric and Pennate diatoms. Particular examples of useful diatoms include: all species within the genus Nitzschia, such as Nitzschia acicularis, Nitzschia alba, Nitzschia alexandrina, Nitzschia amphibian, Nitzschia apiculata, Nitzschia closterium, Nitzschia communis, Nitzschia compressa, Nitzschia dissipata, Nitzschia distans, Nitzschiafiliformis, Nitzschia fonticola, Nitzschiafrustulum, Nitzschia fusiformis, Nitzschia inconspicua, Nitzschia laevis, Nitzschia lecointei, Nitzschia linearis, Nitzschia longissima, Nitzschia navis-varingica, Nitzschia palea, Nitzschia paleacea, Nitzschia paleaformis, Nitzschia pelllucida, Nitzschia pusilla, Nitzschia sigma, Nitzschia supralitorea, Nitzschia thermalis, Nitzschia thermaloides, Nitzschia vitrea; all species within the genus Pinnularia, including Pinnularia quadratare and Pinnularia rupestris; and all species of Centric diatoms, but particularly Cyclotella species, since the cellular biology and biochemistry for biosilification in Centric diatoms is identical for all practical purposes to that for Pennate diatoms. A person of ordinary skill in the art will appreciate that any and all possible combinations of suitable diatom species also can be used.

### V. Production Method

The exemplary embodiments of the production method refer to the diatoms as an example of a biological system. Because diatoms assimilate silica to produce a frustule, a soluble source of silicon is a normal material for diatom growth. Hence, certain embodiments of the disclosed method provide a soluble source of silicon to the diatom and to allow the diatom to grow on the silicon source. This growth period typically is continued until the soluble source of silicon is substantially exhausted and silicon starvation occurs. Silicon starvation can be measured by determining silicon concentration change versus time in the cultivation medium using any suitable method, such as spectrophotometry. If desired, silicon starvation also can be cross-checked. One method for verifying silicon starvation is by determining cell density, i.e. number of cells/unit volume of culture suspension, or the mass of the cells/unit volume of culture suspension. When the cell density is constant with time, and the silicon concentration in the liquid medium is near zero, then silicon starvation is verified. Another method of verifying silicon consumption by the diatom is by direct measurement of the mass of silicon within the measured diatoms cell mass.

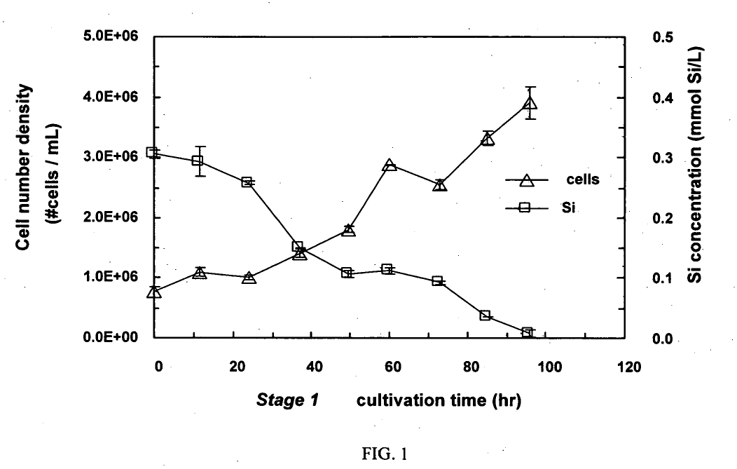

In a specific example, a photosynthetic N. frustulum cell suspension was grown on nutrient medium containing, Si(OH)₄ (silicic acid) as the growth-limiting substrate within a bubble-column photobioreactor. The cultivation was carried out until substantially all the soluble silicon was consumed (see FIG. 1). Once all the soluble silicon was consumed, cell division ceased, the culture was silicon starved, and the diatom was prepared to incorporate a suitable substrate material.

To produce the secondary metal oxide material, a suitable soluble substrate source, such as aqueous germanic acid, Ge(OH)₄, was provided to the diatom and at least one cell division allowed to proceed to harness the nanoscale biomineralization machinery to fabricate silicon-metal oxide nanostructures. An aqueous soluble silicon source, e.g., silicic acid [Si(OH)₄], was co-administered with the substrate. Substrate additions were carried out in working embodiments within a pH range of from about 7.5 to about 9.0, to present the substrates as aqueous tetrahedral metal hydroxides to the silica transporter of the diatom cell. Silicon and/or substrate depletion can be measured as a function of time to determine material synthesis progress. Concurrently, silicon and substrate content in the produced diatom cell mass can be measured. Aliquots can be removed during cultivation so that decreased silicon and/or substrate concentration in the solid biomass can be determined over time.

Another useful facet of the present invention is that the composition of the primary and/or secondary material(s) formed can be varied, as desired, and can be externally controlled during the cultivation step. Desired composition modifications thus can be achieved by manipulating pertinent factors associated with substrate addition, including but not necessarily limited to, any combination of the following: type of substrate, for example aqueous soluble metal hydroxides of silicon, gallium, germanium, and titanium; pulse versus continuous addition of substrate to the culture suspension; initial concentration of substrate; initial total amount of substrate; multiple pulse additions of substrate(s) with constant time intervals; multiple pulse additions of substrate(s) with varying time intervals; rate of continuous substrate addition, either by varying concentration of substrate in feed stream and/or by varying the volumetric flow rate of substrate solution to culture suspension; timing of substrate addition within a given phase of the cell cycle, for example, before or during DNA synthesis (G1, S phase), during active substrate uptake (G2 phase), after silicon starvation (G2 phase), after cell wall synthesis (G2+M phase). Pertinent environmental factors include, but are not limited to: pH of culture medium (pH range of 6 to 10); temperature of cultivation, typically from about 5° C. to 40° C.; illumination of culture suspension (incident light intensity, photoperiod); addition of cell metabolism inhibitors, including, but not limited to 2,4-dinitrophenol, N-ethylmaleimide; addition or deletion of micronutrients in cultivation medium, including, but not limited to the elements B, Co, Cu, Fe, Mn, Mo, Zn and vitamins B1, B12, biotin; addition or deletion of cations from cultivation medium, including, but not limited to K⁺, Na⁺, Ca⁺⁺, Mg⁺⁺. Mass ratios of silicon to substrate can vary from substantially no assimilation for primary materials to substantially complete assimilation for metal oxides comprising substrate metals to the substantial exclusion of silicon. Working embodiments have had an elemental mass ratio (Si:Ge) range of about 42:1 to about 9:1 within the air dried biomass (green powder).

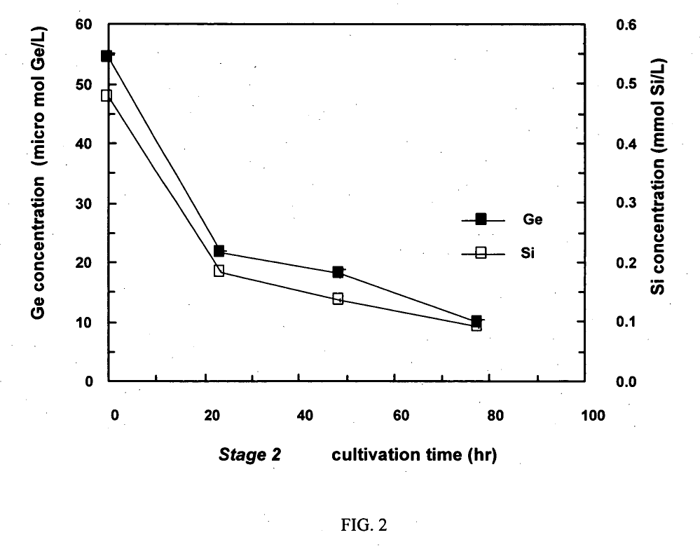

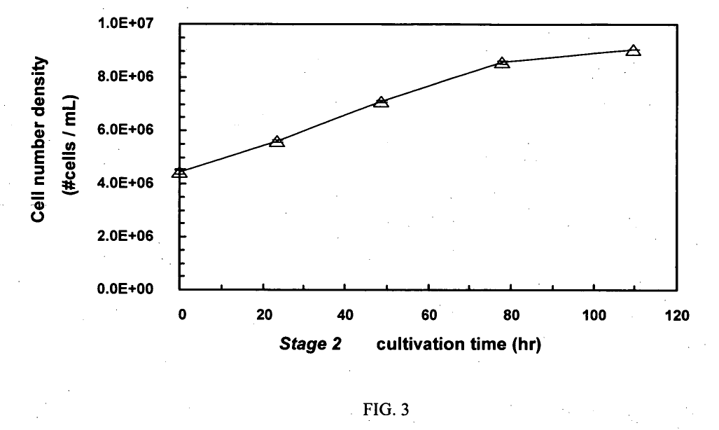

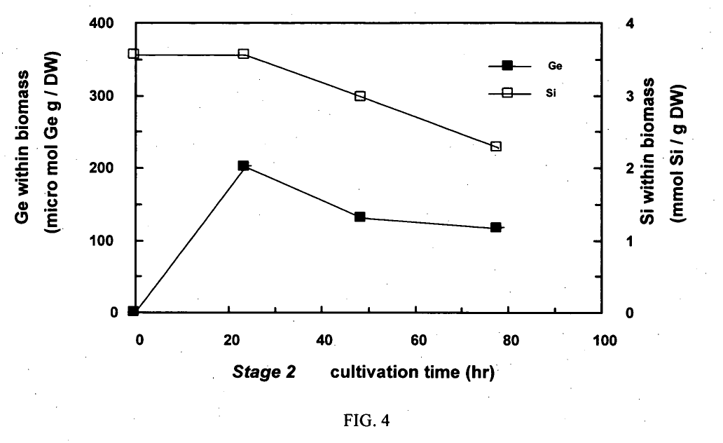

Again in a specific example, a mixture of Ge(OH)₄ (germanic acid) and Si(OH)₄ (silicic acid) dissolved in liquid culture medium was added to silicon-starved cell culture to an initial concentration of 0.6 mM Si and 0.030 mM Ge. The silicon-starved diatom cells co-assimilated soluble silicon and germanium by a surge uptake mechanism, and then underwent one cell division (FIG. 2). After one cell division (FIG. 3), cells bearing a new frustule containing both silicon and germanium oxides were formed. Silicon and germanium concentration can be determined as a function of time (FIG. 4). When Ge(OH)₄ alone without Si(OH)₄ (silicic acid) was added to silicon-starved cell culture, the diatom cells initially took up the germanium but then secreted it back out to the liquid medium. If allowed to continue, the final diatom cell mass did not contain any germanium.

Cultivation of the biological system, such as with a substrate material, is a low-temperature process relative to known commercial processes that are used to make metal oxides. For example, cultivation of the biological system typically was conducted at a temperature of less than 100° C., more typically from above 0° C. to less than about 50° C., even more typically from about 5° C. to about 40° C., and most typically at a temperature of about 20° C.

Desired metal oxides preferably, but perhaps not necessarily for subsequent utilization, are isolated from the other material produced by the organism and/or present-in the cultivation medium, referred to herein as biomass. A combination of physical separation/purification techniques, such as decanting, centrifugation, filtration, and/or drying, and/or chemical processes, can be used to isolate desired materials from the biomass. Most of the superfluous biomass material is organic-based, which can be oxidized substantially completely to produce carbon oxides, such as carbon dioxide, separable from desired inorganic materials. A person of ordinary skill in the art will appreciate that a number of organic oxidizing materials are potentially useful for this step. Such material must be capable of oxidizing substantially completely organic material away from the inorganic material, but also should not change (unless desired, such as to a higher oxidation state), or destroy the inorganic metal oxide material produced by the organism. Solely by way of example, and without limitation, suitable oxidizing materials and/or techniques include oxygen gas, oxygen plasma, aqueous hydrogen peroxide, aqueous permanganates, aqueous concentrated mineral acids (such as sulfuric, nitric and/or hydrochloric) and combinations of such materials and/or techniques. Working embodiments of the present invention typically have used aqueous hydrogen peroxide as the oxidizing agent.

Oxidizing conditions also can vary. For example, oxidizing agent concentration, ratio of oxidizing agent to diatom cell mass, temperature, pressure, agitation rate, and/or pH can be varied to facilitate the process, but preferably are as moderate as possible. For example, oxidation can occur at a temperature of about ambient to at or about the boiling point of the oxidizing material. For a working hydrogen peroxide embodiment the processing temperature was about 60° C. The diatom cell mass from the cultivation process typically is dried, such as drying in air at room temperature or at an elevated temperature, e.g., up to at least about 80° C., for a period of time effective to substantially dry the cell mass, such as a period of about 24 hours. The drying process forms a green powder. The green powder is then chemically treated with an oxidizing agent. Working embodiments typically have used aqueous hydrogen peroxide as an oxidizing agent [e.g., 30 wt % aqueous hydrogen peroxide (typically 30 mL of aqueous hydrogen peroxide to 10 mg dried diatom biomass)]. Working embodiments using aqueous hydrogen peroxide typically were conducted at 60° C. for 24-48 hours using orbital shaking at 100 rpm to oxidize biological and organic materials away from the inorganic frustule. Alternatively, undried cell biomass, or so called wet biomass, can be treated in the same way. The suspension was then centrifuged and washed in deionized water, perhaps plural times, to obtain a final pellet that was collected and then dried in air at room temperature or at 80° C. for 24 hours.

This general procedure typically yields an amorphous white powder containing primarily a mixture of metal oxides, primarily silicon and germanium oxides when the substrate is a material comprising germanium. Working embodiments that use a germanium substrate have had an elemental mass ratio range (Si:Ge) in the amorphous white powder of from about 9:1 to about 42:1. This material possessed both a microstructure characteristic of the frustule and a nanostructure characteristic of the nanoparticles, which self-assemble to form the frustule microstructure.

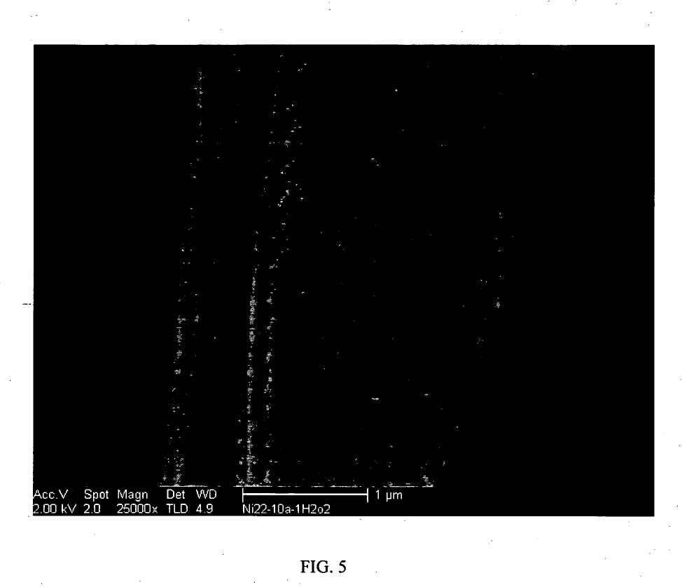

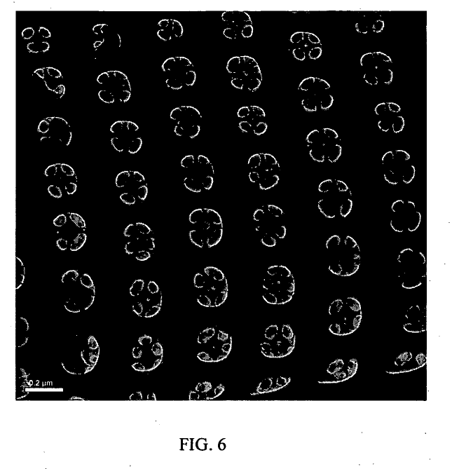

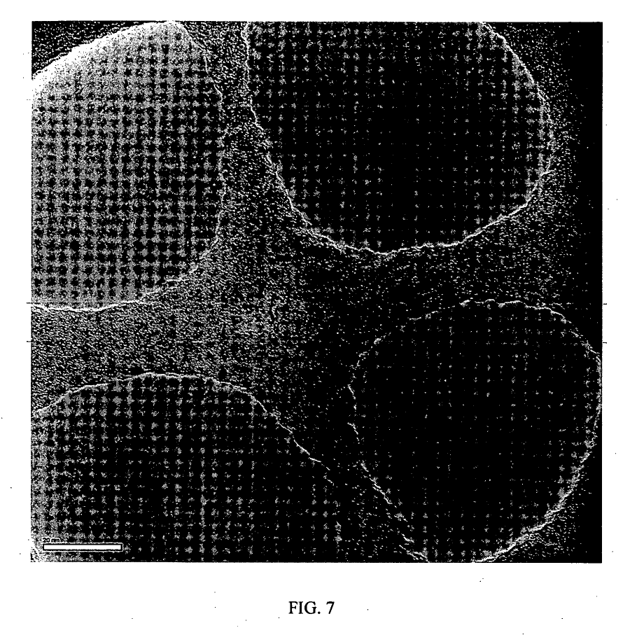

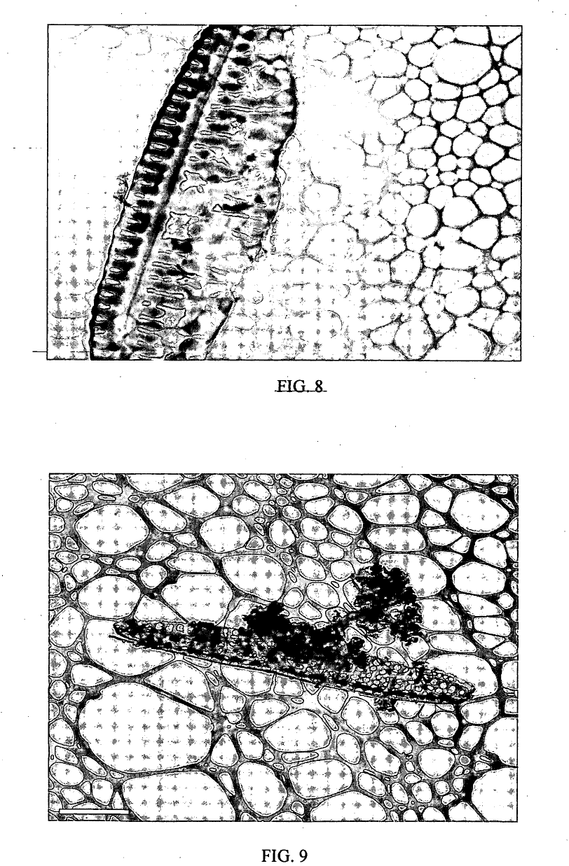

The final nano- and microstructures of the frustule bearing the silicon and germanium oxides may depend upon the amount of germanium and silicon added to the cultivation process. At low germanium loadings, such as from greater than zero percent to at least about 0.6 weight percent in the amorphous white powder, germanium was incorporated into the frustule, but the frustule morphology was altered, leading to a change in microstructure of the material relative to the frustule containing no germanium (FIGS. 5-8). At high germanium loadings of about 2.0 weight percent in the amorphous white powder, the frustule did not maintain its native morphology and instead formed nanoparticles bearing silicon and germanium oxides (FIG. 9). Thus, by manipulating the substrate concentration available to the biological system, nanoclusters, or microstructures assembled from the nanoclusters, can be formed as desired.

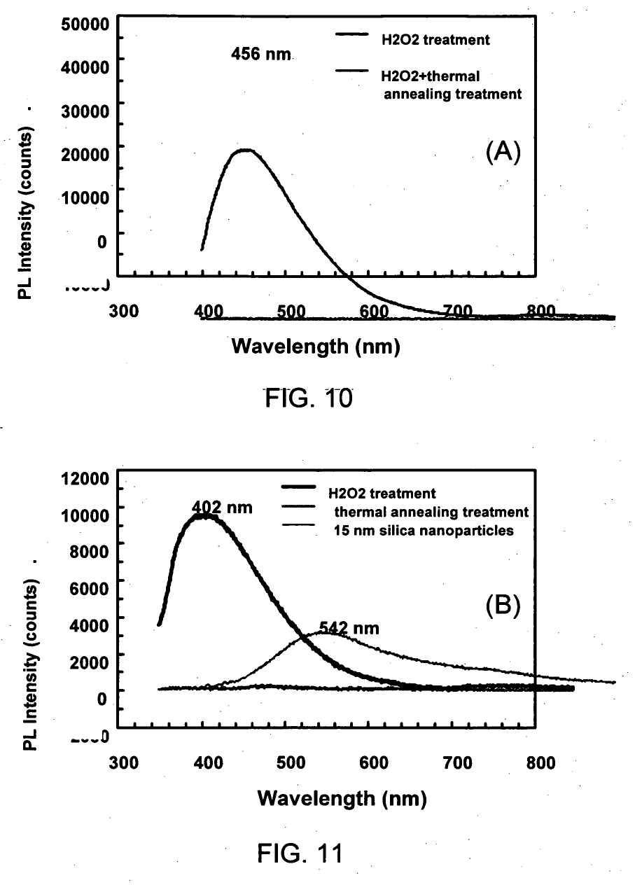

Biogenic metal oxides described above possess optoelectronic properties. In particular, biogenic metal oxides prepared from aqueous hydrogen peroxide treatment of Nitzschia frustulum diatom cell mass that contained a mixture of the primary metal oxide silica and secondary metal oxides of germanium possess blue photoluminescence. For example, the photoluminescence spectrum of Nitzschia frustulum diatom frustules isolated by aqueous hydrogen peroxide treatment that contained 0.25 wt % Ge in the frustule is presented in FIG. 10. This material possessed strong blue photoluminescence with local emission maxima around 456 nm. However, if this same material was further thermally annealed in air at 800° C. for 3 hours, then the photoluminescence disappeared (FIG. 10). The photoluminescence spectrum of Nitzschia frustulum diatom frustules isolated by aqueous hydrogen peroxide treatment that contained 0.50 wt % Ge in the frustule is presented in FIG. 11. This material possessed strong blue photoluminescence with local emission maxima around 402 nm. Direct thermal annealing of the Nitzschia frustulum diatom cell mass in air at 800° C. for 3 hours without the intermediate aqueous hydrogen peroxide step also quenched the photoluminescence (FIG. 11). Bulk silica nanoparticles (15 nm) did not possess any blue photoluminescence, only abroad, relatively weak green-red photoluminescnece centered at 542 nm (FIG. 11). Therefore, the blue photoluminesnce is attributable to the biogenic metal oxides produced by diatoms that were isolated by aqueous hydrogen peroxide treatment.

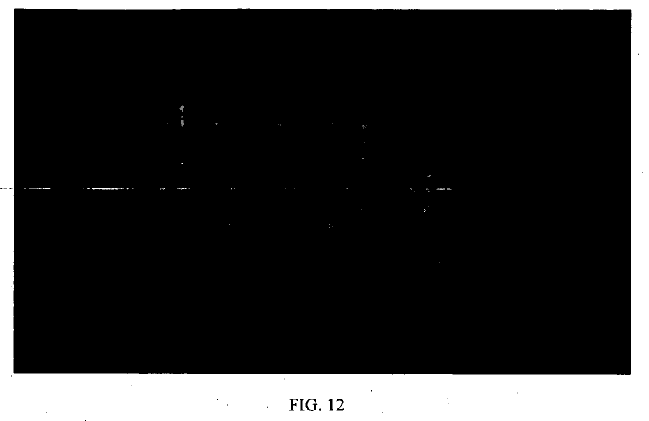

The biogenic, nanostructured Si—Ge oxide powder was used to make the Oregon State University (OSU) logo, which exhibited strong blue photoluminescence under UV light (FIG. 12).

### VI. Post Processing

Diatom cell mass produced by the cultivation process that contains primary or secondary materials can be thermally annealed to make an inorganic material containing metal oxide(s). For example, thermal annealing of dried diatom cell mass (green powder) in air at 800° C. for six hours yields a crystalline white powder primarily containing metal oxides.

Primary or secondary metal oxide materials produced by the biological system followed by chemical oxidation or thermal annealing can also be chemically reduced to their corresponding metallic elements, for example, by reaction with hydrogen gas or a mixture of hydrogen and nitrogen gas at 1.0 atm and 1,000° C.

Primary or secondary metal oxide materials also can be chemically processed after initial production using a first biological system. For example, Si—Ge oxide materials are blue-light emitters, but the color emission can be altered by post processing to produce any desired color. For example, green, red, orange, yellow and white light emitters have been made by producing a primary or secondary material and then adding other elements or materials, or reacting with other elements or materials. For green light emitters, primary silicate materials were produced biologically, and then combined with effective amounts of zinc and manganese to produce Mn²⁺-doped zinc silicate (Zn₂SiO₄:Mn) phosphors, which serve as a green-emitting phosphors in many industrial applications. White light emitters have been made by first producing Si—Ge oxides, and then overcoating such materials with yellow phosphors.

Chemical bath deposition (CBD) is one post processing method that has been applied. CBD is an aqueous analogue of chemical vapor deposition, whereby constituent ions are dissolved in a solvent, and thin films are produced through a heterogeneous surface reaction. Successive ionic layer adsorption and reaction (SILAR) is a second example of a post processing technique. SILAR processes proceed via a layer-by-layer growth mechanism that is similar to atomic layer deposition. SILAR involves immersing a substrate first in a solution containing a desired metal cation. The product is then rinsed, and then immersed in a solution containing the desired anion, and again rinsed. This process ideally provides one monolayer of the deposit per cycle. The process is then repeated for as many cycles as needed.

The following are examples, without limitation, of composite phosphor materials that can be made by such processes: ZnS:Mn/SiO₂:Ge, ZnS:Tb/SiO₂:Ge, ZnS:Sm,Cl/SiO₂:Ge, ZnS:TbOF/SiO₂:Ge, ZnS:Tm,F/SiO₂:Ge, CaS Eu/SiO₂:Ge, CaS:Ce,Cl/SiO₂:Ge, CaSSe:Eu/SiO₂:Ge, SrS:Ce,F/SiO₂:Ge, SrS:Ce,Mn,Cl/SiO₂:Ge, SrS:Ce/SiO₂:Ge, SrS:Ce/SiO₂:Ge, Eu/SiO₂:Ge, ZnO:Ga/SiO₂:Ge, ZnO:Al/SiO₂:Ge, ZnO:Cu/SiO₂:Ge, ZnSe:Cu/SiO₂:Ge, ZnSe:Cu,Cl/SiO₂:Ge, ZnSe:Mn/SiO₂:Ge, ZnTe:Mn/SiO₂:Ge, ZnTe:Eu/SiO₂:Ge, CdS:Ag/SiO₂:Ge, CdS:Te/SiO₂:Ge, CdS:Ln/SiO₂:Ge, CdS:Te/SiO₂:Ge, CdSe:Cl/SiO₂:Ge, CdSe:Ag/SiO₂:Ge, CdTe:Ag/SiO₂:Ge, CdTe:Cl/SiO₂:Ge, CdO:Mn/SiO₂:Ge, CdO:Tm/SiO₂:Ge, CaS:Ce/SiO₂:Ge, CaS:Eu/SiO₂:Ge, CaS:Pb/SiO₂:Ge, CaS:Sm/SiO₂:Ge, CaSe:Ce/SiO₂:Ge, CaSe:Mn/SiO₂:Ge, CaSe:Tm/SiO₂:Ge, SrS:Ce/SiO₂:Ge, SrS:Eu/SiO₂:Ge, SrS:Mn/SiO₂:Ge, SrSe:Ba/SiO₂:Ge, SrSe:Ce/SiO₂:Ge, BaS:Au/SiO₂:Ge, BaS:Ce/SiO₂:Ge, BaS:Mn/SiO₂:Ge, BaSe:Eu/SiO₂:Ge, BaSe:Mn/SiO₂:Ge, LaPO₄:Eu/SiO₂:Ge, LaPO₄:Ce/SiO₂:Ge, CePO₄:Eu/SiO₂:Ge, CePO₄:Ce/SiO₂:Ge, GdPO₄:Tb/SiO₂:Ge, GdPO₄:Ce/SiO₂:Ge, YPO₄:Eu/SiO₂:Ge, YPO₄:Mn/SiO₂:Ge, YPO₄:V/SiO₂:Ge, ScBO₃:Tb/SiO₂:Ge, ScBO₃:Eu/SiO₂:Ge, ScBO₃:Ce/SiO₂:Ge, YBO₃:Eu/SiO₂:Ge, YBO₃:Ce/SiO₂:Ge, LaBO₃:Eu/SiO₂:Ge, LaBO₃:Mn/SiO₂:Ge, CeBO₃:Eu/SiO₂:Ge, CeBO₃:Tb/SiO₂:Ge, PbMoO₄:Eu/SiO₂:Ge, PbMoO₄:Mn/SiO₂:Ge, CaMoO₄:Eu/SiO₂:Ge, CaMO₀₄:Ce/SiO₂:Ge, CdMoO₄:Eu/SiO₂:Ge, CdMoO₄:Mn/SiO₂:Ge, ZnMoO₄:Eu/SiO₂:Ge, ZnMoO₄:Ce/SiO₂:Ge, CdMoO₄:Mn/SiO₂:Ge, BaMoO₄:Mn/SiO₂:Ge, BaMoO₄:Eu/SiO₂:Ge, BaMoO₄:Tb/SiO₂:Ge, Gd₂(MoO₄):Mn/SiO₂:Ge, Gd₂(MoO₄):Tb/SiO₂:Ge, YVO₄:Eu/SiO₂:Ge, YVO₄:Dy/SiO₂:Ge, YPO₄:Eu/SiO₂:Ge, YPO₄:Mn/SiO₂:Ge, YPO₄:Mn,Th/SiO₂:Ge, CaWO₄:Sm/SiO₂:Ge, CaWO₄:Pb/SiO₂:Ge, PbWO₄:Tb/SiO₂:Ge, and PbWO₄:Ce/SiO₂:Ge.

Nanostructured silicate with tunable luminescent properties also can be made using embodiments of the present invention. Silicate-based materials are an important class of inorganic phosphors that are useful for many commercial applications such as cathode ray tubes (TVs, PC monitors, test equipment), fluorescent lighting, and more recently light emitting diodes, paints, and glazes. Three-dimensional nanostructured silicate-based phosphors with tunable luminescent colors can be made using the presently disclosed methods. For example, chemical bath deposition has been used to deposit a thin layer of metal oxides over the nanostructured diatom silica frustule. Such materials have then been converted into silicate-based phosphors by regular thermal annealing, or a lower temperature hydrothermal annealing process. A working embodiment of a hydrothermal annealing process comprised placing precursors (e.g. ZnCl₂ and MnCl₂) and nanostructured diatom powder (SiO₂) at a pre-calculated stochiometry into a Teflon hydrothermal reactor. A desired amount, such as just a few drops, of deionized water was also added to the reaction. The reactants were then heated, such as by placing the hydrothermal reactor into an oven at an effective temperature, typically about 200° C., and for an effective period of time, typically about 24 hours, for working embodiments. During the annealing process, saturated water vapor water converts the silica into desired silicates.

The following are some examples, without limitation, of potential silicate-based phosphor materials that could be made by disclosed process embodiments: CaSiO₃:Ce, CaSiO₃:Eu, CaSiO₃:Pb, CaSiO₃:Ti, CaSiO₃:Pb,Mn, Be₂SiO₄:Mn, Mg₂SiO₄:Mn, Zn₂SiO₄:Mn, Zn₂SiO₄:Mn,P, Zn₂SiO₄:Mn,As, Zn₂SiO₄:Ti, (Zn+Be)₂SiO₄:Mn, Sr₂SiO₄:Eu, SrBaSiO₄:Eu, Ba₂SiO₄:Eu, Ba₂SiO₄:Ce,Li,Mn, BaSi₂₅:Eu, BaSi₂₅:Pb, Y₂SiO₅:Ce, CaMgSi₂O₆:Eu, CaMgSi₂O₆:Eu,Mn, Ca₂MgSi₂O₇:Eu, Ca₂MgSi₂O₇, Ca₂MgSi₂O₇:Eu,Mn, Sr₂MgSi₂O₇:Eu, Ba₂MgSi₂O₇:Eu, BaMg₂Si₂O₇:Eu, BaSrMgSi₂O₇:Eu, Ba₂Li₂Si₂O₇:Eu, Ba₂MgSi₂O₇:Sn, Ba₂MgSi₂O₇:Sn,Mn, MgSrBa₂Si₂O₇:Eu, MgBa₃Si₂O₈:Eu, MgSr₃Si₂O₈:Eu,Mn, Sr₃MgSi₂O₈:Eu, Ca₅B₂SiOIO:Eu, Ca₃Al₂Si₃O₁₂:Eu, LiCeBa₄Si₄O₁₄:Mn, LiCeSrBa₃Si₄O₁₄:Mn.

### VII. Devices and Applications

Materials made according to the present invention can be used in the construction of a variety of devices, and can be used for a number of applications. With reference to device applications, the materials can be used to make optoelectronic devices, including memory and data storage, capacitors, detectors, sensors, lasers, emitters and light-emitting diodes, optical computing architectures, photonics (e.g. waveguides), photovoltaics (e.g. solar cells), photocatalysts for promoting chemical oxidation reactions, etc. A person of ordinary skill in the art of making such devices will understand how such devices can be constructed using disclosed compositions.

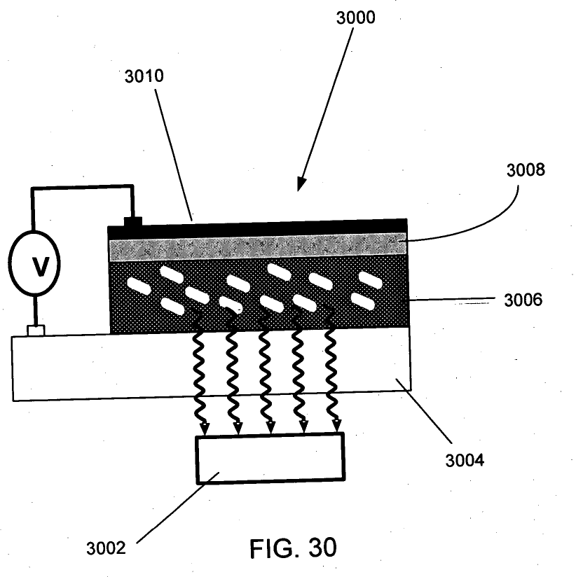

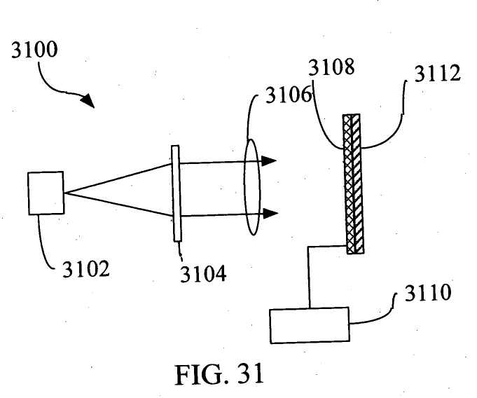

For example, one embodiment of an electroluminescent device 3000 that can be fabricated using materials made according to the present invention is illustrated in FIG. 30. Device 3000 includes a CCD detector 3002, an indium tin oxide (ITO) layer 3004, a diatom frustule layer 3006, a dielectric layer 3008 and a conductive layer 3010. In this embodiment, layer 3006 comprises a thin film of diatom frustules positioned between a transparent, electrically conducting ITO glass layer 3004. IN working embodiments, layer 3004 was about 300 nanometers thick. Device 3000 also included a 100 nanometer thick hafnium oxide thin film layer as the dielectric layer 3008. Conductive layer 3010 was a thin film of aluminum, such as a layer of about 100 nanometers thick. Conductive layer 3010 was deposited over a hafnium oxide layer 3008. When a voltage is applied across the device from the aluminum condctive layer 3010 to the ITO electrode layer 3004, the diatom layer 3006 emits light at discrete wavelengths.

More complex displays can also be provided. A representative addressable display 3100 based on luminescence is illustrated in FIG. 30A. The display 3100 includes an illumination source 3102 that is typically configured to provide radiation in a ultraviolet or near ultraviolet wavelength range, typically between about 200 nm and about 450 nm, but other wavelengths can be used. For example, visible radiation (400 nm to 700 nm) can be used. An illumination optical system 3104 receives radiation from the illumination source 3102 and forms an illumination beam 2706 that is directed to a liquid crystal panel (LCP) 2708. The illumination beam 3106 is typically collimated or at least partially collimated for delivery to the LCP 3108. The LCP 3108 typically defines an array of independently switchable liquid crystal regions (referred to herein as LCP display pixels) with a series of row and column electrodes. Typically the LCP 3108 includes one or more polarizers and a liquid crystal layer, but these and other features of the LCP 3108 are not shown in FIG. 30A. Display electronics 3110 are coupled to the LCP 3108 and selectively activate one or more display pixels of the LCP 3108 to permit a corresponding portion of the illumination beam 3106 to propagate through the LCP 3108 and reach a luminescent layer 3112. Portions of the luminescent layer 3112 that are activated by the illumination beam 3106 appear substantially brighter than un-activated portions. The display electronics 3110 can provide various image signals to the LCP 3108, such as, for example, video signals or computer display signals, and the resulting luminescence can be either viewed directly or imaged with additional optics.

Images formed by the display 3100 can be in one or more colors based on colors produced in the luminescent layer 3112. For example, the luminescent layer can include materials that luminesce at each of three primary colors (such as red, green, and blue), and these materials can be patterned for independent excitation by radiation transmitted by the LCP 3108. In one example, red, green, and blue luminescent portions are patterned in the layer 3112 to form luminescent display pixels wherein each portion is associated with a corresponding LCP display pixel.

Disclosed compositions also can be used in medical imaging applications. Typically, prior to annealing, the biologically produced metal oxides are both amorphous and non-crystalline. These two factors facilitate both medical imaging applications, as well as drug delivery applications. For medical imaging, non-crystalline materials can be administered to a subject with substantially less discomfort than crystalline materials, simply from considering the sharper edges, points, etc. defined by crystalline materials relative to amorphous materials. Second, these materials can be produced as nanoclusters, and hence as administered are quite small, which again facilitates administration. After administration, material excitation, such as light absorption, can be initiated by an energy source external to the subject. With reference to photoluminescent materials, upon absorption, such materials can thereafter emit detectable light for imaging purposes.

These materials also can be used for therapeutic applications, such as drug delivery or phototherapy, by forming a composition comprising an active agent or mixture of active agents. A nanostructured metal oxide typically is first produced. For example, nanoparticles of the metal oxide imbedded within a defined microstructure of the diatom frustule may be used. One embodiment comprises biologically making a frustule consisting of a uniform array of pores that consists of nanostructured primary or secondary luminescent metal oxide. Such metal oxide or semimetal oxide material(s) optionally may be post processed. A therapeutic composition is formed, comprising at least one active agent and the metal oxide(s). For example, an effective amount of the active agent may be physically admixed with a metal oxide material. As a second example, active agent may be loaded into a frustule microstructure, such as a uniform array of frustule pores. An amount of a composition effective to image, diagnose or treat a disease or malady is then administered to a subject. The composition may comprise an amount of the active agent effective to treat a particular disease or malady, and an amount of a metal oxide, such as a nanostructured metal oxide, effective to facilitate delivery of the active agent, such as light facilitated active agent delivery.

Embodiments of the present invention also can be formulated with a light-sensitive delivery material, such as a light sensitive liposome. Light-sensitive delivery materials and photochemotherapy applications are known. See, for example, Cytosolic drug delivery using pH- and light-sensitive liposomes, Gerasimov O. V et al., Advanced Drug Delivery Reviews, Volume 38, Number 3, 20 August 1999, pp. 317-338(22); A novel tetracationic phthalocyanine as a potential skin phototherapeutic agent, Fabris, Clara et al., Experimental Dennatology, Volume 14, Number 9, September 2005, pp. 675-683(9); and A polymeric drug delivery system for the simultaneous delivery of drugs activatable by enzymes and/or light, Krinick et al., J. Biomater Sci. Polym. Ed., 5(4):303-24 (1994). Each of these publications is incorporated herein by reference.

Therapeutic compositions incorporate a diagnostic or therapeutic agent. Examples of suitable diagnostics or therapeutics, without limitation, can be selected from the group consisting of nucleic acids, proteins, naturally occurring organic compounds, synthetic and semi-synthetic compounds, and combinations thereof. More particularly, the diagnostic or therapeutic agent may be an AIDS adjunct agent, alcohol abuse preparation, Alzheimer's disease management agent, amyotrophic lateral sclerosis therapeutic agent, analgesic, anesthetic, antacid, antiarythmic, antibiotic, anticonvulsant, antidepressant, antidiabetic agent, antiemetic, antidote, antifibrosis therapeutic agent, antifungal, antihistamine, antihypertensive, anti-infective agent, antimicrobial, antineoplastic, antipsychotic, antiparkinsonian agent, antirheumatic agent, appetite stimulant, appetite suppressant, biological response modifier, biological, blood modifier, bone metabolism regulator, cardioprotective agent, cardiovascular agent, central nervous system stimulant, cholinesterase inhibitor, contraceptive, cystic fibrosis management agent, deodorant, diagnostic, dietary supplement, diuretic, dopamine receptor agonist, endometriosis management agent, enzyme, erectile dysfunction therapeutic, fatty acid, gastrointestinal agent, Gaucher's disease management agent, gout preparation, homeopathic remedy, hormone, hypercalcemia management agent, hypnotic, hypocalcemia management agent, immunomodulator, immunosuppressive, ion exchange resin, levocarnitine deficiency management agent, mast cell stabilizer, migraine preparation, motion sickness product, multiple sclerosis management agent, muscle relaxant, narcotic detoxification agent, narcotic, nucleoside analog, non-steroidal anti-inflammatory drug, obesity management agent, osteoporosis preparation, oxytocic, parasympatholytic, parasympathomimetic, phosphate binder, porphyria agent, psychotherapeutic agent, radio-opaque agent, psychotropic, sclerosing agent, sedative, sickle cell anemia management agent, smoking cessation aid, steroid, stimulant, sympatholytic, sympathomimetic, Tourette's syndrome agent, tremor preparation, urinary tract agent, vaginal preparation, vasodilator, vertigo agent, weight loss agent, Wilson's disease management agent, and mixtures thereof.

Particular examples of such therapeutics and diagnostics include, without limitation, abacavir sulfate, abacavir sulfate/lamivudine/zidovudine, acetazolamide, acyclovir, albendazole, albuterol, aldactone, allopurinol BP, amoxicillin, amoxicillin/clavulanate potassium, amprenavir, atovaquone, atovaquone and proguanil hydrochloride, atracurium besylate, beclomethasone dipropionate, berlactone betamethasone valerate, bupropion hydrochloride, bupropion hydrochloride SR, captopril, carvedilol, caspofingin acetate, cefazolin, ceftazidime, cefuroxime (no sulfate), chlorambucil, chlorpromazine, cimetidine, cimetidine hydrochloride, cisatracurium besilate, clobetasol propionate, co-trimoxazole, colfosceril palmitate, dextroamphetamie sulfate, digoxin, enalapril maleate, epoprostenol, esomepraxole magnesium, fexofenadine, fluticasone propionate, furosemide, gancyclovir, hydrochlorothiazide/triamterene, lamivudine, lamotrigine, lithium carbonate, losartan potassium, melphalan, mercaptopurine, mesalazine, metformin, methyldopa, minocycline, mupirocin calcium cream, nabumetone, naratriptan, omeprazole, ondansetron hydrochloride, orli stat (or a pharmaceutically acceptable salt thereof), ovine, oxiconazole nitrate, paroxetine hydrochloride, prochlorperazine, procyclidine hydrochloride, pyrimethamine, ranitidine bismuth citrate, ranitidine hydrochloride, rofecoxib, ropinirole hydrochloride, rosiglitazone maleate, salmeterol xinafoate, salmeterol, selegiline, fluticasone propionate, sterile ticarcillin disodium/clavulanate potassium, simvastatin, spironolactone, succinylcholine chloride, sumatriptan, thioguanine, tirofiban HCI, topotecan hydrochloride, tranylcypromine sulfate, trifluoperazine hydrochloride, valacyclovir hydrochloride, vinorelbine, zanamivir, zidovudine, zidovudine, lamivudine, and combinations thereof.

Effective amounts of the diagnostic or therapeutic agent may be incorporated into the composition in the form of a solution, suspension, emulsion, tablet, capsule, powder, bead, pellet, granules, solid dispersion, or combinations thereof. Optionally, the diagnostic or therapeutic agent may be more soluble in gastric fluid than intestinal fluid, more soluble in intestinal fluid than gastric fluid, better absorbed within small intestine than within large intestine, better absorbed within stomach than within intestines, or better absorbed within intestines than within stomach.

Effective therapeutic compositions also can include other agents or combinations of agents useful for making compositions, including for example, an excipient, such as a plasticizer, a pH adjuster, a GI motility adjuster, a viscosity adjuster, an expansion agent, a surfactant, or mixtures thereof.

A plasticizer can be added to the composition to increase the plasticity of the mixture to a level suitable for administering to a subject. Plasticizers may be hydroxylated compounds, particularly poly-hydroxylated organic compounds. For example, polyethylene glycol (PEG) is a poly-aliphatic hydroxylated organic compound that has been used in working examples. Persons skilled in the art could substitute other plasticizers, for example glycerin or surface-active materials. Typically, working embodiments have included from about 1% to 8% plasticizer.

A pH adjuster can be added to adjust the pH to a desired pH level. Suitable pH adjusters include buffers, mineral acids or bases, or organic acids or bases. The pH adjuster is optionally a buffer. Other pH adjusters are known to those of skill in the art, and can include, without limitation, hydrochloric acid, sodium hydroxide, potassium hydroxide, organic acids, such as acetic acid, and organic amines, particularly lower (10 carbon atoms or fewer) alkyl amines, such as triethylamine, and combinations thereof.

### VIII. EXAMPLES

The following examples are provided solely to illustrate working embodiments of the present invention. The scope of the invention is not limited to those features described in such examples.

Example 1

This example concerns one embodiment of a method for making metal oxides by cultivating diatoms and post-processing the diatom cell mass into mixed primary metal oxide materials produced by the thermal annealing method. Example 1 specifically concerns cultivation of Nitszschia frustulum diatoms in the presence of germanium, the preparation of a metal oxide solid by direct thermal annealing of the cultivated diatom cell mass, and the subsequent chemical characterization of the metal oxide solid produced. Although Example 1 specifically concerns Pennate Nitzschia frustulum diatoms, the processing information provided applies equally well to other types of diatoms including, for example, other Pennate diatoms, such as Pinnularia sp. or Centric diatoms such as Cyclotella Pinnularia.

The pennate, photosynthetic marine diatom Nitzschia frustulum was obtained from the UTEX culture collection of Algae (UTEX # 2042) and is described by Lewin and Lewin. J. C. Le win and R. A. Le win, Auxotrophy and heterotrophy in marine littoral diatoms. Can. J. Microbiol. 6, 127 (1960). Diatom cell suspension cultures were grown in natural seawater medium (OSU Marine Science Center, Newport, Oreg.) enriched with Lewin's marine diatom medium (LDM), as modified by the UTEX Culture Collection of Algae (media/ldm.html), but without supplemental tryptone, NaCl or CaCl₂. The culture medium was supplemented with 4.0 mM nitrate, 0.19 mM phosphate, and 0.53 mM Si(OH)₄, so that culture growth was ultimately limited by Si. Stock cultures were maintained without agitation in 500-mL flasks (100 mL per flask) at 22° C., 125 μ Em⁻² s⁻¹ incident light intensity, and 14:10 L:D photoperiod. Subculture was performed every 2 weeks at 10% v/v.

A 3-L, bubble column photobioreactor described by Huang and Rorrer [Y. M. Huang and G. L. Rorrer, Dynamics of oxygen evolution and biomass production during cultivation of Agardhiella subulata microplantlets in a bubble column photobioreactor under medium perfusion, Biotechnol. Progr. 18, 62 (2002), incorporated herein by reference] and Polzin and Rorrer [J. J. Polzin and G. L. Rorrer, Halogenated monoterpene production by microplantlets of the marine red alga Ochtodes secundiramea within an airlift photobioreactor under nutrient medium perfusion, Biotechnol. Bioeng. 82, 415 (2003), incorporated herein by reference] was used for all phototropic diatom cultivation experiments. External illumination to the 7.6-cm inner diameter, glass bioreactor vessel was provided by four vertically mounted, 15-W fluorescent lamps, positioned to uniformly deliver 75 μ Em⁻² s⁻¹ incident light intensity to the vessel. The illumination photoperiod was fixed at 14 hours light/10 hours dark. The vessel temperature was maintained at 22° C. with a water jacket surrounding the vessel that was connected to a circulating water bath. Air containing 350 ppm CO₂ was filter sterilized (0.2 μm), humidified and then introduced into the bottom of the vessel with a 4.0-cm diameter, 60-μm fritted-glass sparger. The volumetric gas flow rate was fixed at 0.5 L of air L⁻¹ of culture minute⁻¹, which was sufficient to uniformly suspend the diatom cell suspension. Germanium was incorporated into the diatom cell mass by a two-stage photoreactor cultivation process.

In stage 1, the diatom cell suspension was grown on Si(OH)₄ (silicic acid) in batch culture. At the time when all the Si in the culture medium was consumed, the diatom cells were considered “Si starved.” At this time, Ge(OH)₄ or a mixture of Ge(OH)₄ and Si(OH)₄ was added to the diatom suspension. The maximum solubility of silicic acid and germanic acid are 1.0 and 50 mM, respectively, at 25° C. Furthermore, speciation diagrams show that the predominant soluble species of Si and Ge in water at pH 6 to 9 and 25° C. are the neutral (aq) species Si(OH)₄ (IV) and Ge(OH)₄ (IV). Process conditions for the two-stage bioreactor cultivation experiments are detailed in Table 1, below.

TABLE 1
Process parameters for two-stage photobioreactor
cultivation of N. frustulum.

| | Ge pulse | Ge + Si |
|---|---|---|
| Process parameter | addition | pulse addition |
| Initial Si concentration | | |
| Stage 1 | 13.5 mg of Si/L | 11.8 mg of Si/L |
| | (0.481 mM) | (0.348 mM) |
| Stage 2 | 0.0 mg of Si/L | 7.31 mg of Si/L |
| | (0.0 mM) | (0.252 mM) |
| Initial Ge concentration | | |
| Stage 2 | 7.45 mg of Ge/L | 1.60 mg of Ge/L |
| | (0.103 mM) | (0.022 mM) |
| Cultivation time at | 103 h | 95 h |
| stage 2 | | |
| Final Ge uptake into | 4.83 ± 0.07 mg | 2.74 ± 0.04 mg |
| biomass (stage 2) | of Ge/g of DCW | of Ge/g of DCW |
| Specific growth rate | | |
| Stage 1 | 0.023 h⁻¹ | 0.038 h⁻¹ |
| Stage 2 | 0.0 h⁻¹ | 0.0087 h⁻¹ |

To initiate stage 1 of the cultivation process, the bioreactor was typically inoculated with 2×10⁵ diatom cells/mL in natural seawater supplemented with modified LDM nutrients (no tryptone, NaCl or CaCl₂), 4.0 mM nitrate, 0.19 mM phosphate, and 0.35 mM Si(OH)₄ as the growth-limiting substrate. The pH of the aerated medium was between 8.2 and 8.4. Under these conditions, the, specific growth rate of the cells during stage 1 was typically within 0.02-0.04 h⁻¹, and Si consumption from the culture medium was complete within 100-140 hours. To initiate stage 2 of the cultivation process, soluble Ge(OH)₄ alone or a mixture of soluble Ge(OH)₄ and Si(OH)₄ was added to the culture. No other process variables were changed.

Culture samples (25 mL) were aseptically removed from the bioreactor vessel at appropriate time intervals. A 20-mL portion of the sample was immediately filtered on a 3.0-μm Versapor acyclic filter (Pall Life Sciences) to separate the liquid medium from the cells and quench any further Ge or Si uptake. The liquid was then assayed for Ge and Si concentration. The remaining sample volume (5 mL) was assayed for cell number density. At selected times during Ge uptake, 300-mL culture suspension samples also were removed to provide approximately 300 mg of fresh cell mass for later materials characterization studies. Dry cell mass, cell number density, soluble Ge concentration, and soluble Si concentration also were assayed.

Cell number density was assayed by manual hemocytometer cell count (Fuchs-Rosenthal, 300-μL sample chamber, no dilution). Cell mass was determined gravimetrially, where 20-mL samples were filtered, washed with deionized water, and oven dried in air at 80° C. for 24 hours. Soluble silicon in the culture medium was assayed spectrophotometrically at 410 nm using methods described by Marczenko. Z. Marczenko, Spectrophotometric Determination of the Elements, Ellis Horwood Ltd./John Wiley & Sons, New York (1976). Soluble germanium in the culture medium also was assayed spectrophotometrically at 525 nm using methods described by Marczenko. The Ge and Si concentration in the biomass was assayed by first digesting the fresh biomass with sodium hydroxide (NaOH). Specifically, 50 mg fresh biomass was mixed with one gram solid NaOH and heated within a zirconium crucible at 400° C. for 25 minutes. The mixture was cooled to room temperature, resuspended in 20 mL deionized water and then neutralized with 10 wt % nitric acid to pH 7. The Si and Ge concentration in the neutralized solution was assayed spectrophotometrically as described above, and the Si or Ge content within the biomass (as mg/g biomass or mmol/g biomass) was calculated from the Si or Ge concentration, the final solution volume, and the weight of biomass. All assays were carried out in duplicate.

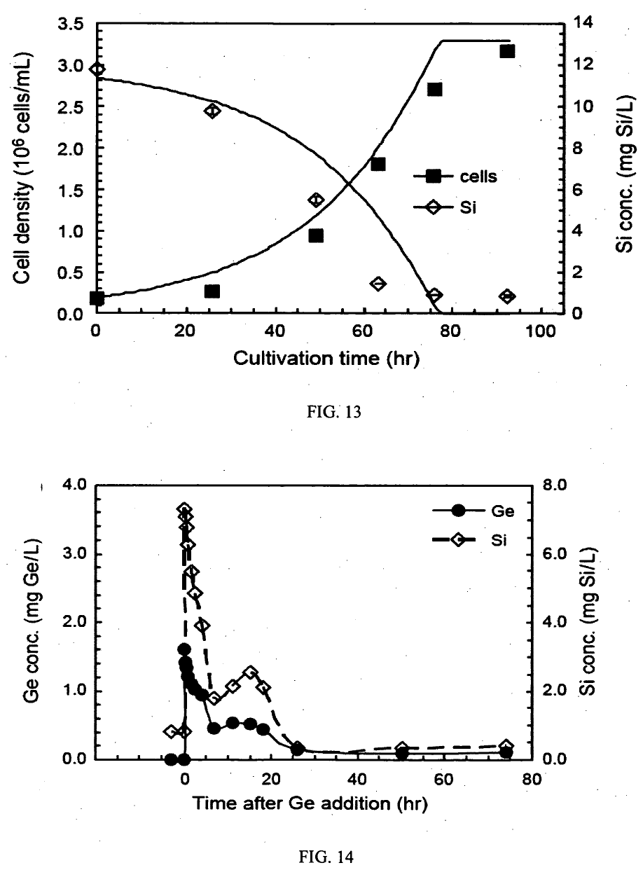

Results from the two-stage photobioreactor cultivation of N. frustulum diatom suspension are presented in FIGS. 10 and 11. Process parameters for Si and Ge input during photobioreactor cultivation are detailed in Table 1. In stage 1 of the cultivation process, N. frustulum diatoms were grown up on soluble Si(OH)₄ to the point of Si starvation. In stage 2 of the cultivation process, soluble Ge(OH)₄ or a mixture of soluble Ge(OH)₄ and Si(OH)₄ was added to the Si-starved diatom suspension. Two cultivation processes were conducted. First, a mixture of soluble Ge(OH)₄ and Si(OH)₄ was added, where the Ge was at a relatively low concentration. Second, soluble Ge(OH)₄ alone at a relatively high concentration was added. The Ge uptake versus time profiles during stage 2 for each experiment are presented in FIGS. 12 and 13.

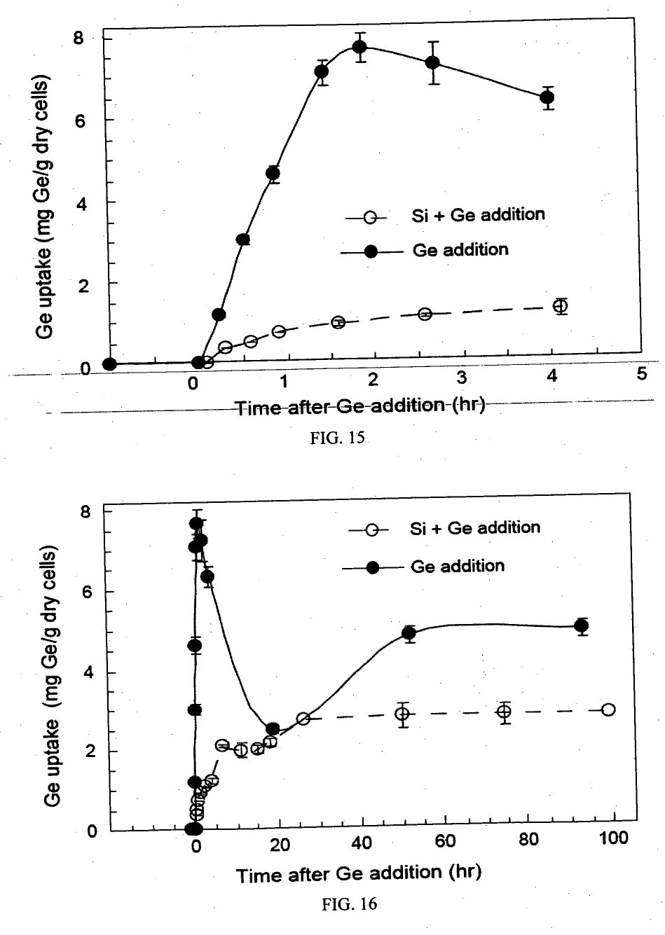

For the pulse addition of a mixture Ge and Si at an initial concentration 1.6 mg of Ge/L and 7.31 mg of Si/L (0.022 mM Ge and 0.25 mM Si, for a Si:Ge M ratio of 11.8), the uptake process proceeded to an equilibrium condition. Ge uptake into the cells was rapid during the first 5 hours following Ge addition, suggesting Ge was assimilated by a “surge uptake” process normally reserved for Si-starved cells. C. W. Sullivan, Diatom mineralization of silicic acid. I. Transport characteristics in Navicula pelliculosa, J. Phycol. 12, 390 (1976). C. W. Sullivan, Diatom mineralization of silicic acid. II. Regulation of Si(OH)₄ transport rates during the cell cycle of Navicula pelliculosa, J. Phycol.13, 86 (1977). In the time period from 5 to 26 hours following Ge+Si pulse addition, there was a small “burp” in both Ge uptake and Si uptake before equilibrium was established (FIG. 14). A final Ge uptake of 2.74±0.04 mg of Ge/g of DCW was obtained 26 hours after Ge+Si addition. Ge uptake paralleled Si uptake, and the concentration of both metals went to zero in culture liquid medium. Diatom cells also continued to divide, but at a much lower rate. For stage 1, the cell division rate was 0.038 h⁻¹, but for stage 2, the specific growth rate was only 0.0087 h⁻¹, a factor of 4 lower. For pulse addition of soluble Ge(OH)₄ alone at an initial concentration of 7.45 mg/L (0.103 mM Ge), the “surge uptake” of Ge also was observed during the first 5 hours (FIG. 15). This surge uptake of Ge was followed by a release of Ge back to the culture medium, followed again by slower re-uptake of Ge that proceeded to equilibrium, ultimately yielding a cell mass containing 4.83±0.07 mg of Ge/g of DCW 92 hours after Ge addition (FIG. 16). Following the pulse addition of Ge alone, no further diatom cell division was observed. However, by microscopic observations, the diatoms cells remained viable at least 123 hours after Ge addition to the culture suspension.

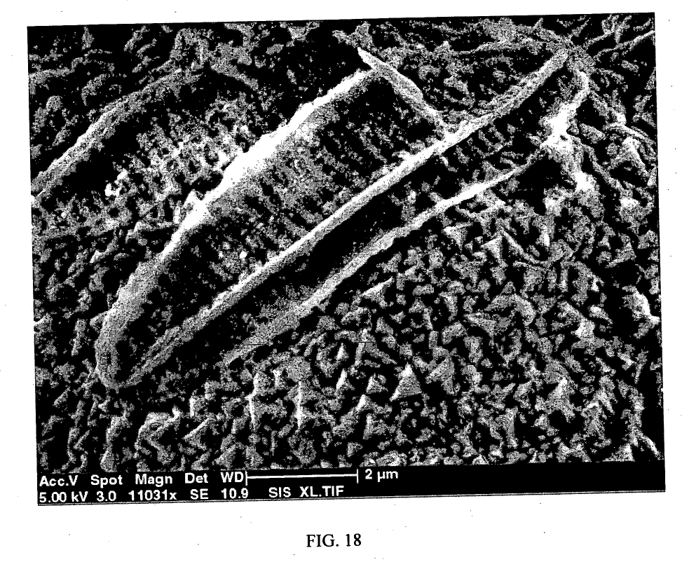

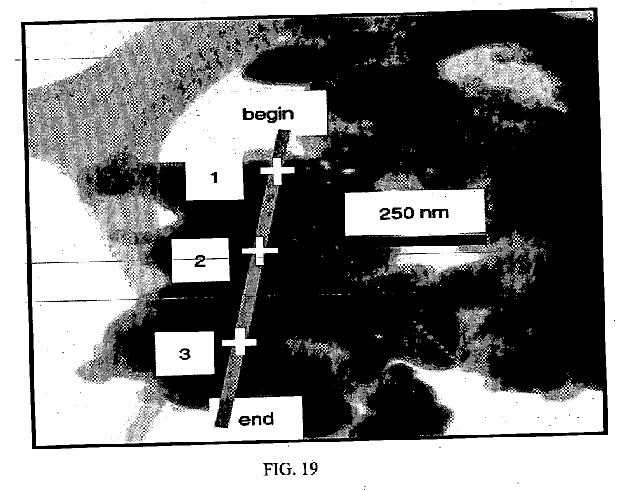

Previous attempts at characterizing Ge assimilation by the heterotrophic Nitzschia alba via ⁶⁸Ge radiolabeling [F. Azam, B. B. Hemmingsen, and B. E. Volcani, Germanium incorporation into the silica of diatom cell walls, Arch. Mikrobio, 92, 11 (1973); F. Azam and B. E. Volcani, Role of silicon in diatom metabolism. VI. Active transport of germanic acid in the heterotrophic diatom Nitzschia alba. Arch. Mikrobiol.101, 1 (1974)] never considered long-term uptake studies beyond 24 hours. However, long-term uptake facilitates synthesis of composite nanomaterials by the diatom cell. Spectroscopic methods, including FT-IR, X-ray diffraction (XRD), and energy dispersive X-ray spectroscopy (EDS), corroborated that a mixture of silicon, germanium, and calcium oxides was formed during stage 2 of the cultivation process (FIGS. 19-26). These analyses were facilitated by thermally annealing the diatom cell mass. The procedures and results are detailed below.

The diatom cell mass was thermally annealed to yield a white crystalline powder. The air-dried diatom cell mass (35-mg aliquot), composed of a coarse green powder, was evenly spread out within a nickel crucible and heated in a furnace for 6 hours at 800° C. in still air. During thermal annealing in air, carbonaceous material in the cell mass oxidized to CO₂, yielding a fine, residual white powder enriched in the biogenic inorganic material. Thermally annealed samples were stored in a desiccating chamber at room temperature.

Transmission electron microscopy (TEM) and energy-dispersive X-ray spectrometry (EDS) analyses were performed on the thermally annealed cell mass. The thermally annealed cell mass, consisting of a white powder, was ground in mortar and pestle to break apart the microscopic diatom shells. This ground powder was then resuspended in acetone and pipetted onto a copper TEM grid coated with a Lacey carbon film. These samples were observed by transmission electron microscopy (TEM) at 200 KeV using a Tecnai F20 field emission TEM equipped with an embedded scanning transmission electron microscope (STEM) and EDS at Portland State University, Portland, Oreg. Once a representative nanocluster was identified by TEM, EDS analysis in line-scanning mode was performed (200 KeV, 15-nm increment, 5000 ms per increment). Characteristic energy peaks were identified as 0.52 KeV for O, 1.21 KeV for Mg, 1.74 KeV for Si, 3.67 KeV for Ca, and 9.80 KeV for Ge. Selected points along the line scan were selected for quantitative elemental analysis.

FT-IR Spectroscopy also was performed. A Nicolet 510P FT-IR spectrbphotometer equipped with DTGS detector (4 cm⁻¹ resolution) was used to obtain Fourier-transform infrared (FT-IR) spectra of the thermally annealed cell mass. An aliquot of the thermally annealed cell mass (typically 2 mg) was ground in 100 mg of KBr to a fine powder, and 40 mg of this mixture was loaded into the FT-IR cell. FT-IR analyses were performed in transmission mode.

An X-ray diffraction (XRD) spectrum of the thermally annealed cell mass was obtained on a Siemens D5000 X-ray diffractometer with a Cu K αradiation source at 2θ resolution of 0.02°.

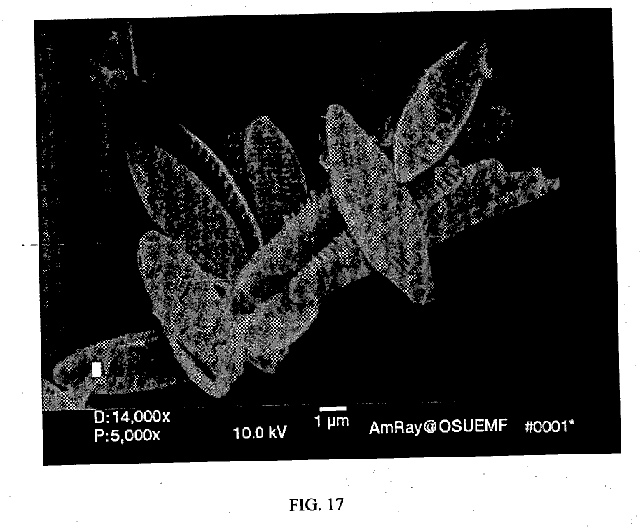

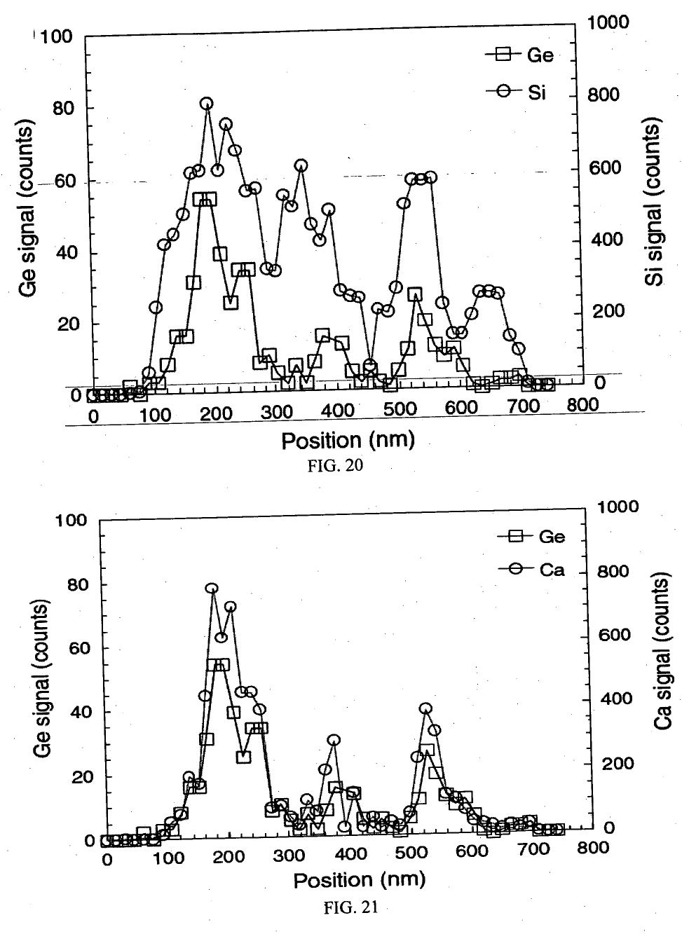

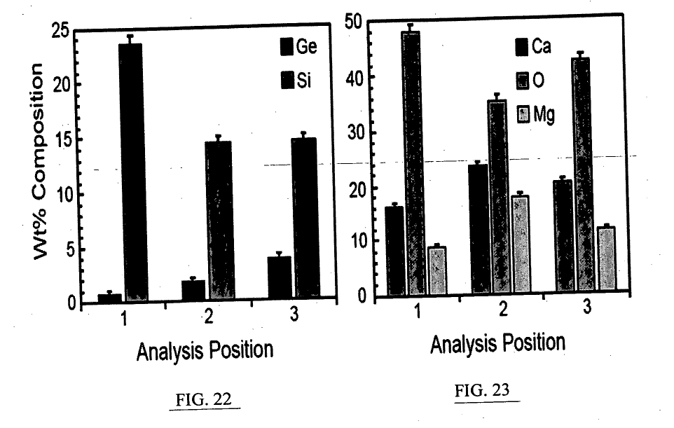

An SEM of the thermally annealed N. frustulum frustules is presented in FIG. 17. The cell mass, obtained 123 hours after pulse addition of 7.45 mg of Ge/L to the Si-starved diatom cell suspension culture, was dried in air at room temperature and then thermally annealed in air at 800° C. for 6 hours to remove carbonaceous materials, yielding a white powder. High magnification SEM imaging of this powder showed that the diatom shell remained intact after thermal annealing (FIG. 18). This material was fractured by mechanical grinding, and nanocluster fragments released from the thermally-annealed diatom shells were analyzed by TEM-EDS (FIG. 19). A TEM-EDS line scan through a representative nanocluster revealed nanostructured phases of Si and Ge (FIG. 20). The material with the darker contrast tended to be richer in Ge. Interestingly , the periodic nanostructure of Ge was intimately associated with Ca (FIG. 21). Quantitative elemental analysis of the nanocluster at these three points showed that 95-98% of the elemental constituents of the white powder were Si, Ge, Ca, Mg, and O (FIGS. 22 and 23). The atomic composition of oxygen was sufficient to ensure that all metals were in an oxide form. Only minor amounts of carbon (less than 0.3 wt %) and phosphorous (less than 1.5 wt %) were found at these three points. The composition of the bulk material could not be quantified by TEM-EDS because copper (Cu) and carbon (C) from the TEM grid showed up in the analysis when the whole sample was profiled.

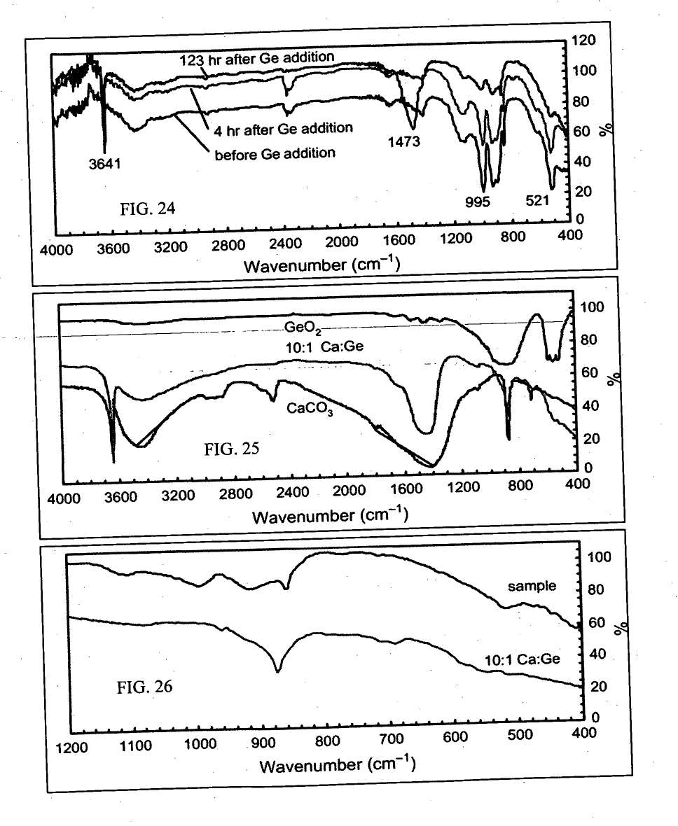

FT-IR Spectroscopy FT-IR analyses of the thermally annealed cell mass following the pulse addition of 7.45 mg of Ge/L to the Si-starved diatom cell suspension culture are presented in FIGS. 24-26. Before the addition of soluble Ge to the diatom suspension culture, the biogenic inorganic material in the cell mass contained mainly silicon oxides. The four peaks at 1138, 995, 925/895, and 845 cm⁻¹ correspond to SiO₄ tetrahedral structural units with nonbridging O per Si (NBO/Si) of 1, 2, 3, and 4, respectively, whereas the peak at 521 cm⁻¹ represents the Si—O bending mode. Furthermore, this FT-IR spectrum did not reveal the presence of SiO₂—CaO mineral phases. Four hours after Ge addition, the uptake of soluble Ge by the diatom cell suspension culture was near its maximum (see FIGS. 15 and 16), but the FT-IR spectrum of the thermally annealed biomass was similar to that of the thermally annealed cell mass before Ge addition, indicating that germanium oxides were not yet formed. However, 123 hours after Ge addition, the FT-IR spectrum of the thermally annealed cell mass was markedly different, as many peaks characteristic of SiO₂ disappeared. Furthermore, sharp peaks appeared at 3,641 and 1,473 cm⁻¹, which are indicative of Ca—O bonds associated with thermal annealing of biogenic amorphous calcium carbonate, e.g., aragonite, from calcareous marine algae. For comparison, FT-IR spectra for standards of crystalline GeO₂, crystalline CaCO₃, and a thermally annealed (800 ° C. for 6 hours in air) mixture of a 10:1 M ratio of CaCO₃ to GeO₂ are presented in FIG. 25. IR spectra of SiO₂/GeO₂ glass mixtures also are given by Maragaryan and Piliavin. A. Maragaryan and M. A. Piliavin, Germanate Glasses, Artech House, Boston (1993). The presence of free GeO₂ in the thermally annealed cell mass could not be corroborated by FT-IR. However, the FT-IR spectrum of the thermally annealed 10:1 M CaCO₃/GeO₂ standard was similar to that of the thermally annealed cell mass sample obtained 123 h after Ge addition (FIG. 26).

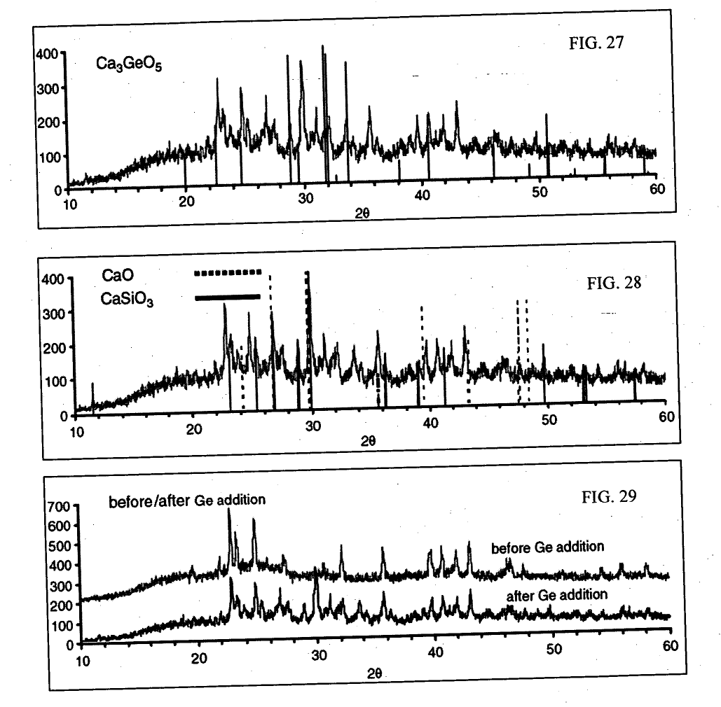

XRD analysis of the thermally annealed cell mass, obtained 123 hours after pulse addition of a mixture of 1.60 mg of Ge/L and 7.31 mg of Si/L to the Si-starved diatom cell suspension culture, is presented in FIGS. 27-29. Key signals matched standards for crystalline mineral phases of Ca₃GeO₅, CaSiO₃, and CaO (FIGS. 24 and 25). The XRD analysis of thermally annealed diatom cell mass just before pulse addition of Ge+Si to the diatom cell suspension culture lacked characteristic signals for CaSiO₃ and Ca₃GeO₅ (FIG. 29).

TEM revealed that frustule fragments from thermally annealed N. frustulum biomass consisted of nanoclusters (FIG. 16). Elemental profiling by TEM-EDS line scanning of these frustule fragments also revealed that the germanium-rich mineral phases were nanostructured, with features ranging from about 20 to about 100 nanometers in size (FIGS. 19-21). Furthermore, a closer examination of FIGS. 20-21 shows that the Si-rich phases did not always coincide with Ge-rich phases. Thus, Si—Ge oxide nanocomposites with ordered, periodic structures can be made. However, in embodiments that were thermally annealed probably sintered or agglomerated the germaniun nanoclusters [J. G. Zhu, C. W .White, J. D. Budai, S. P. Withrow, and Y. Chen, Growth of Ge, Si, and SiGe nanocrystals in SiO₂ matrices, J. Appl. Phys. 78, 4386 (1995)], suggesting that the true biogenic nanostructure might be even finer than that presented in FIGS. 19-21. It is not known why soluble calcium, which is a major constituent in seawater (10 mM), was incorporated into N. frustulum biomass during stage 2 of the cultivation process. Biogenic CaCO₃ formation is common to many “calcareous” algae, but not within the Bacillariophyceae. Calcium was not found in the thermally annealed N. frustulum biomass before exogenous addition of Ge(OH)₄ to the N. frustulum culture; Ca was found in the biomass only after Ge(OH)₄ addition. Given the intimate association of Ge with Ca on the nanoscale (FIG. 21), calcium assimilation is coupled to Ge assimilation during stage 2 of the cultivation process.

### Example 2

This example concerns one embodiment of a hydrogen peroxide oxidation process for oxidizing biological and organic materials away from the inorganic frustule for the purpose of isolating biogenically produced metal oxides. Diatom cell mass from the cultivation process, such as that described in Example 1, was dried in air at room temperature or at 80° C. for 24-48 hours to produce a green powder. The green powder was then chemically treated with 30 wt % aqueous hydrogen peroxide (typically 30 mL of aqueous hydrogen peroxide to 10 mg dried diatom biomass) at 60° C. for 24 hours. Orbital shaking at 100 rpm also was employed. Alternatively, undried cell biomass, or so called wet biomass, can be treated in the same way. The suspension was then centrifuged. The supernatant was removed, the pellet was resuspended in deionized-distilled water, and then centrifuged again. The final pellet was collected and then dried in air at room temperature or at 80° C. for 24 hours. This procedure yielded an amorphous white powder containing primarily a mixture of metal oxides, primarily silicon and germanium oxides. Working embodiments have had an elemental mass ratio (Si:Ge) range of about 9:1 to about 42:1 in the amorphous white powder. This material possessed both a microstructure characteristic of the frustule and a nanostructure characteristic of the nanoparticles, which self-assemble to form the frustule microstructure.

### Example 3

This example concerns the production of ZnSiO₄:Mn²⁺. Silica was isolated from Nitzschia diatom using hydrogen peroxide oxidation. Hydrothermal annealing was then performed by combining Nitzschia isolated SiO₂ (3.48 mg), ZnSO₄ (0.018g) and MnSO₄ (0.0172g) and water in a teflon vessel reactor (acid bomb reactor). The reactor was placed in an oven and kept at a temperature of 200° C. for 24 hours.

### Example 4

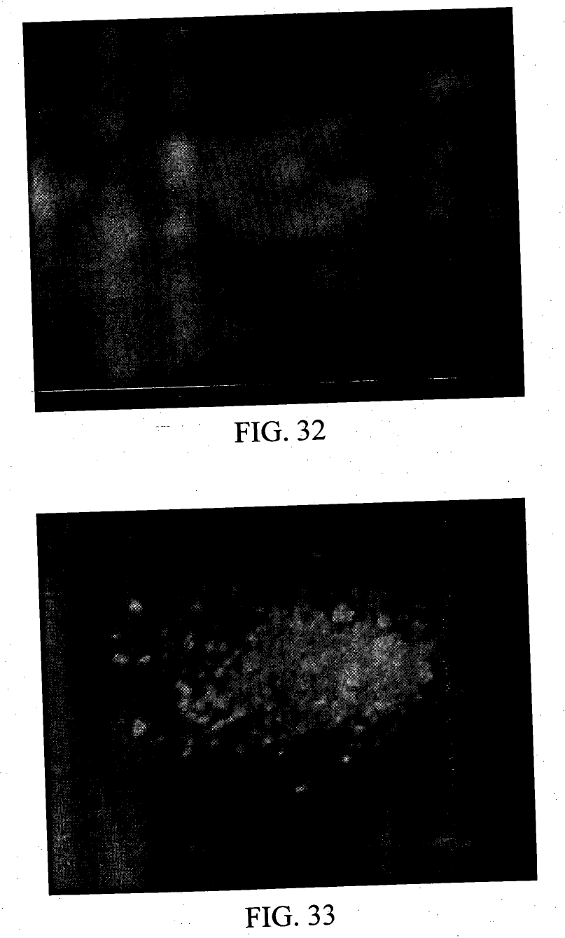

This example concerns the production of ZnS:Mn²⁺/SiO₂:Ge using silica isolated from a Nitzschia diatom by hydrogen peroxide oxidation. SILAR processing was then used. Nitzschia SiO₂ (10 mg) was placed on 2.5 micron filter paper. Then 0.1 M ZnCl₂, 0.1 M Na₂S and 0.005 M MnCl₂ aqueous solutions were prepared as precursors for the SILAR process. In between cation (Zn²⁺) and anion (S²⁻) deposition, deionized water was sprayed on the diatom powder as a rinsing step. The process was repeated for 10 cycles and the resulting material dried at 80° C. Material produced by the method is depicted in FIG. 32.

### Example 5

This example concerns the production of ZnS:Mn²⁺/SiO₂:Ge using a Pinnularia diatom, H₂O₂ oxidation of organic material and SILAR post processing. Pinnularia derived SiO₂ (10 mg) was placed on 2.5 micron filter paper. Then 0.1M ZnCl₂, 0.1M Na₂S and 0.005M MnCl₂ aqueous solutions were prepared as precursors for the SILAR process. In between cation (Zn²⁺) and anion (S²⁻) deposition, deionized water was sprayed onto the diatom powder as a rinsing step. The process was repeated for 10 cycles and dried 80° C. Material produced by the method is depicted in FIG. 33.

### Example 6

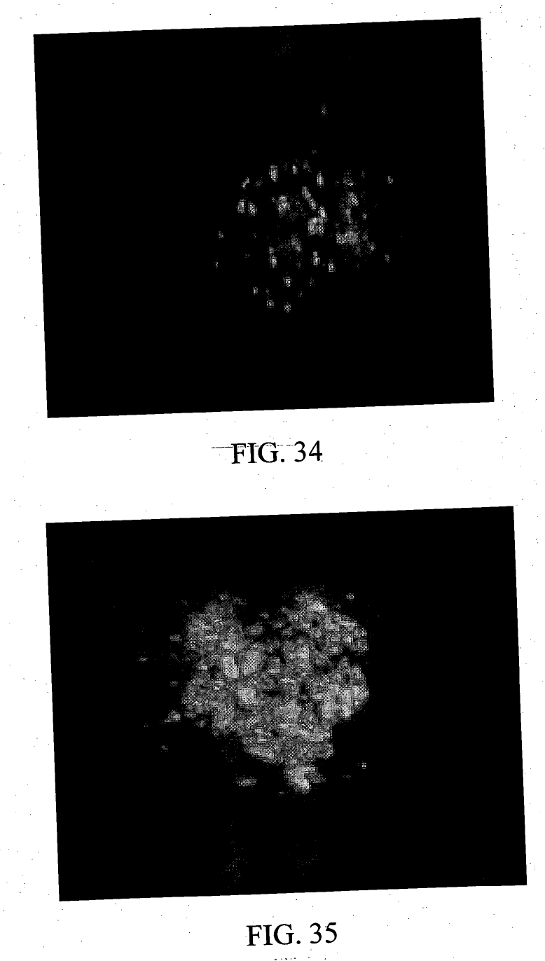

This example concerns the production of ZnSiO₄:Mn²⁺ using silica isolated from a Nitzschia diatom by hydrogen peroxide oxidation and CBD processing. Nitzschia SiO₂ (3.9 mg) and 0.177 g of ZnCl₂ were mixed in 10 ml deionized water. Then 0.17 mg of MnCl₂ was added to the solution. The solution was mixed and heated by a hot plate at 150° C. The powders were filtered out of the solution and annealed in a furnace at 900° C. for 1 hour. Material produced by the method is depicted in FIG. 34.

### Example 7

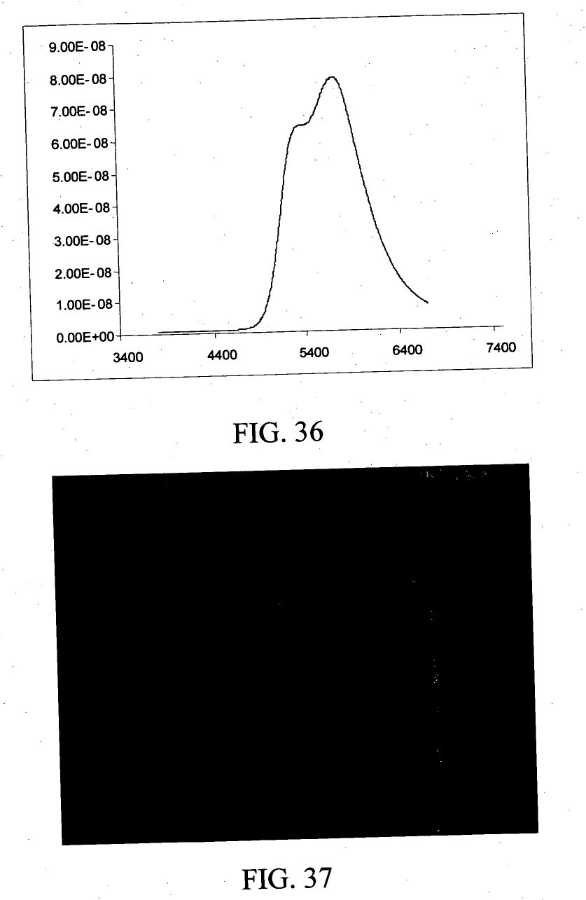

This example concerns the production of ZnSiO₄:Mn²⁺ using silica isolated from a Pinnularia diatom by hydrogen peroxide oxidation and CBD processing. Pinnularia isolated SiO₂ (3.6 mg) and ZnCl₂ (0.16 g) were mixed in 10 ml deionized water. Then 0.17 mg of MnCl₂ was added to the solution. The solution was mixed and heated by a hot plate at 150° C. The powders were filtered out of the solution and annealed in a furnace at 900° C. for 1 hour. Material produced by the method is depicted in FIG. 35, and photoluminescence is indicated by FIG. 36.

### Example 8

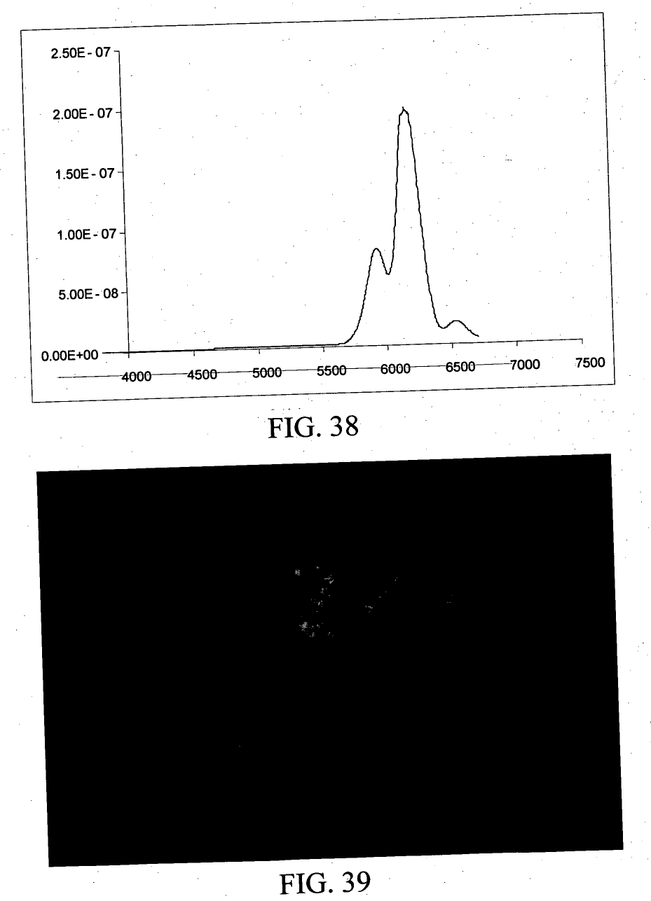

This example concerns the production of Sr₂Si₂O₇:Eu²⁺ using silica isolated from a Pinnularia diatom by hydrogen peroxide oxidation and CBD processing. Pinnularia SiO₂ (10 mg) and SrCl₂ (0.0475 g) were mixed in 10 ml deionized water. Then 0.25 mg EuCl₃ was added to the solution. The solution was mixed and heated by a hot plate at 150° C. The powders were filtered out of the solution and annealed in a furnace at 900° C. for 1 hour. Material produced by the method is depicted in FIG. 37, and photoluminescence is indicated by FIG. 38.

### Example 9

This example concerns the production of Sr₂MgSi₂O₇:Eu²⁺ using silica isolated from a Pinnularia diatom by hydrogen peroxide oxidation CBD processing. Pinnularia SiO₂ (3.8 mg), SrCl₂ (9.7 mg) and MgCl₂ (3 mg) were mixed in 10 ml deionized water. Then 0.25 mg EuCl₃ was added to the solution. The solution was mixed and heated by a hot plate at 150° C. The powders were filtered out of the solution and annealed in a furnace at 900° C. for 1 hour. Material produced by the method is depicted in FIG. 39.

### Example 10

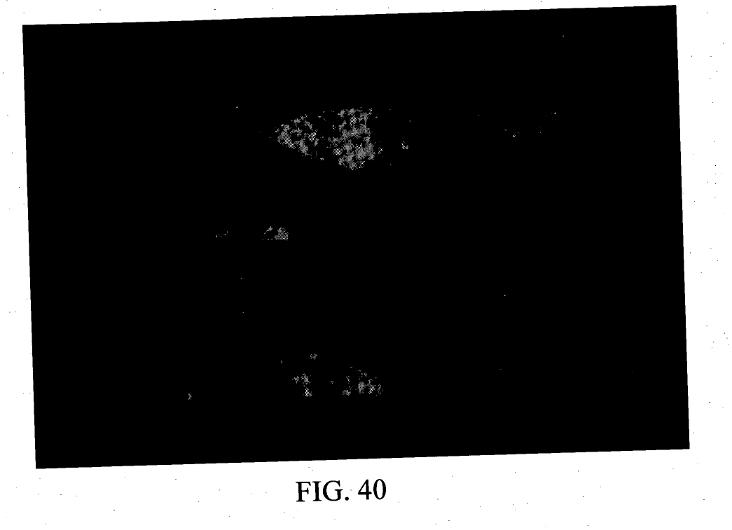

This example concerns the production of SrBaSiO₄:Eu⁺ using silica isolated from a Pinnularia diatom by hydrogen peroxide oxidation and CBD processing. Nitzschia SiO₂ (2.3 mg), BaCl₂ (2.3 mg) and SrCl₂ 4.5 mg were mixed in 10 ml deionized water. Then 0.25 mg EuCl₃ was added to the solution. The solution was mixed and heated by a hot plate at 150° C. The powders were filtered out of the solution and annealed in a furnace at 900° C. for 1 hour. Material produced by the method is depicted in FIG. 40.

### Example 11

This example concerns one embodiment of a method for making metal oxides by cultivating diatoms and post-processing the diatom cell mass into mixed primary and secondary metal oxide materials using an aqueous hydrogen peroxide oxidation treatment method. Example 11 specifically concerns a two-stage photobioreactor process for the metabolic insertion of nanostructured germanium into the intact silica microstructure of the diatom Pinnularia sp, the subsequent isolation of intact frustules possessing altered microstructure that contain nanostructured germanium, and the characterization of the photoluminescence properties and the metal oxide materials produced by these methods. Although Example 11 concerns Pennate Pinnularia sp. diatoms, the processing information provided applies equally well to other types of diatoms including, for example, Centric diatoms such as Cyclotella.

Methods for diatom cell culture maintenance are described below. Pure cultures of the marine diatom Pinnularia sp. (Ehrenberg) were obtained by the UTEX Culture Collection of Algae (UTEX# B679). Pinnularia sp. is a photosynthetic diatom possessing pennate morphology, with a nominal length of 30 μm and width of 5 μm. Stock cultures of Pinnularia sp. were maintained on enriched natural seawater medium without agitation in 500 mL foam-stoppered flasks (80 mL per flask) under cool white fluorescent light at 50 μE/m²-sec incident light flux intensity and photoperiod of 14 hours light/10 hours dark within an incubator set at 22° C. The cell suspension was subcultured every 14 days at 10% v/v. The typical cell density just before subculture was ˜1·10⁶ cells/mL. Filtered, UV-sterilized natural seawater was obtained from the Hatfield Marine Science Center, Newport, Oreg., and re-filtered at 5 μm. The enrichment medium, a modified LDM recipe, (http://www.zo.utexas.edu/research/utex/media/ldm.html), was designed for silicon-limited culture growth with an N:P ratio of 23.4:1. Macronutrients in the enrichment medium were 5.125 mM NaNO₃, 0.0543 mM K₂HPO₄.3H₂O, 0.165 mM KH₂PO₄, 1.03 mM Na₂SiO₃.5H₂O; micronutrient metals were 69.6 μM MgSO₄.H₂O, 3.08 μM FeSO₄.7H₂O, 1.77 μM MnCl₂.4H₂O, 0.314 μM ZnCl₂, 0.072 μM CoCl₂.6H₂O, 0.136 μM (NH₄)₆Mo₇O₂₄.4H₂O, 17.2 μM Na₂EDTA; vitamins were 0.013 μM B₁₂, 0.07 μM biotin, 5.1 μM thiamine HCl, 13.6 μM thymine, 7.2 μM Ca-pantothenate, 1.25 μM p-aminobenozic acid, 95 μM meso-inositol, and 13.9 μM nicotinic acid. The final medium was autoclaved at 121° C. for 30 minutes. All culture manipulations were performed in a laminar flow hood using sterile techniques.

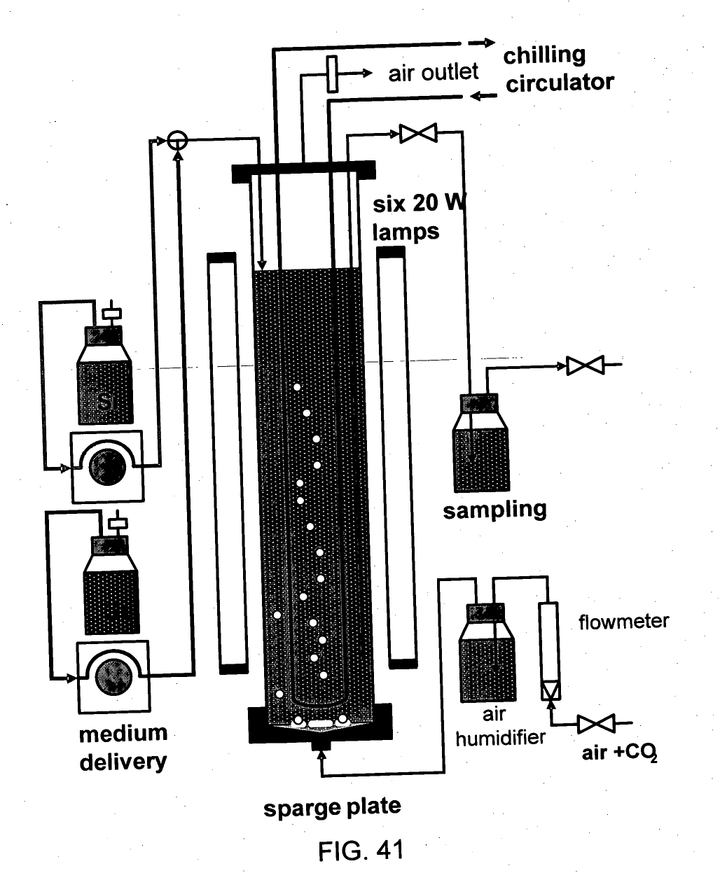

The design and fabrication of the photobioreactor used for mass cultivation of the diatom cell culture is described below. The bubble-column photobioreactor 4100 shown in FIG. 41 was used to cultivate the Pinnularia sp. photosynthetic cell suspension under controlled conditions. The bioreactor vessel 4102 was a glass column of 10.5 cm inner diameter, 4.8 mm wall thickness, and 70.5 cm height to provide a total volume of 6.1 L and a working volume of 3.9 L (45 cm). The glass column 4102 was mounted onto two stainless steel support plates 4104 at the base and top 4106. The baseplate assembly contained a stainless steel sparger plate 4104 having four 1.0 mm diameter holes on a 3.6 cm square pitch. Pressurized house air was particulate filtered, metered through a flowmeter 4108, sterile filtered at 0.2 μm, and introduced to the baseplate 4104. The headplate assembly 4106 contained 8 ports, including a fresh medium delivery port 4110, thermocouple port, a 4.6 mm inner diameter sampling tube, 11 mm D.O. electrode port, two air outlet ports 4112, 4114, and 9.5 mm outer diameter by 1.09 m length stainless steel internal U-tube heat exchanger 4116. Controlled sampling of the liquid suspension within the vessel 4100 was accomplished by pressurizing the vessel headspace and collecting the liquid in a sterile culture bottle 4118. Water from a temperature-controlled chilling circulator 4120 was pumped through the internal heat exchanger 4116 to provide constant temperature within the bioreactor vessel 4102. The bioreactor 4100 was externally illuminated by six 20 W cool white fluorescent lamps 4120 of 57 cm length vertically positioned around the glass vessel 4102 in a hexagonal array about 1-2 cm from the vessel surface. The lamps 4120 were connected to a photoperiod timer (not shown). The incident light flux intensity was measured with a LI-COR SA 190 PAR quantum sensor positioned at the interior surface and pointed towards the light source at 6 radial positions and 3 axial positions (top, middle, bottom). Detailed process conditions used in this study are provided in Table 2. Four identical photobioreactor systems were operated in parallel.

In all bioreactor experiments, medium composition was the same as for the Pinnularia sp. flask cultures with the exception of the dissolved silicon concentration. The medium and bioreactor were autoclaved separately. The bioreactor was inoculated using 17-day-old Pinnularia sp. flask cultures by sterile transfer of a defined inoculum volume. In Stage I of the two-stage photobioreactor cultivation process, culture growth was designed for silicon limitation. The target initial cell density was 7.5·10⁴ cells/mL, the initial pH was 8.3, and the initial dissolved silicon concentration (C_{Si,o}) was 0.53 mmol Si/L (as soluble Na₂SiO₃), to provide a target final cell number density of 6.0·10⁵ cells/mL, or approximately 3 cell divisions. The photoperiod was set to 4 hours illumination, then 10 hours dark, then 14 hours illumination/10 hours dark for the rest of the cultivation period. After all the soluble silicon was consumed, the culture was kept in a silicon-starved state at constant, stationary phase cell density for at least 24 hours.

At the point of silicon starvation, Stage II cultivation was initiated 4 hours into the photoperiod. Specifically, defined volumes of 100 mmol Na₂SiO₃/L and 5.0 mmol GeO₂/L stock solutions in de-ionized water were mixed together and then injected into the bioreactor culture. The initial dissolved silicon concentration was always reset to 0.53 mmol/L, which was enough silicon to provide for one more cell division of silicon-starved cell culture leading to a final cell number density of 1.2·10⁶ cells/mL at the end of Stage II. In Stage II, the initial soluble germanium concentration varied from 0, 4.25, 17.3, 25.2 to 38.3 μmol/L at the fixed initial Si concentration of 0.53 mmol/L, to provide initial Si:Ge molar ratios ranging from 125:1 to 14:1 (initial Si:Ge mass ratios from 48:1 to 5.4:1). The cultivation times for Stages I and II were 114 and 56 hours respectively (170 hours total). During Stage I, 40 mL of the cell suspension was sampled at 12 hours intervals for determination of cell number density, pH, and dissolved silicon concentration. During Stage II, the culture suspension was sampled 8 times during the first 12 hours for cell number density, pH, dissolved silicon concentration, dissolved germanium concentration, and then once every 12 hours thereafter. Additionally, at 0, 12, and 56 hours of Stage II cultivation, 540 mL samples were taken for determination of silicon and germanium content in the dry cell biomass (Y_{Si}, Y_{Ge}), isolation of intact frustules for nanoscale analysis by SEM, TEM, STEM-EDS, and elemental composition of germanium and trace minerals the silica frustule.

To determine cell number density (X_{N}, #cells/mL) a 0.1 mL sample of the cell suspension was diluted in 10.0 mL of Isoton II electrolyte solution (Beckman-Coulter). Cells were counted on Beckman Z2 Coulter Counter at a threshold of 8 microns (duplicate assay). To determine soluble silicon and germanium concentrations, immediately after sampling, a 30 mL sample aliquot of cell suspension was syringe filtered with a cellulose ester membrane (3.0 μm pore size, 25 mm diameter). Soluble silicon concentration (C_{Si}, mmol Si/L) in the filtrate was determined by spectrophotometric assay at 360 nm following derivatization of a 5.0 mL liquid sample aliquot with 0.2 mL of 13.3% w/v ammonium molybdate reagent in water and 0.1 mL 18.7% v/v HCl in water. The soluble germanium concentration (C_{Ge}, μmol Ge/L) in the filtrate was determined by spectrophotometric assay at 510 nm following derivatization of a 1.0 mL liquid sample with 1.0 mL of 0.01% w/v phenylfluorone reagent in methanol, 1.7 mL of 10% v/v HCl in water, 0.3 mL of 24% w/v H₂SO₄ in water, and 1.0 mL buffer (3.3 M sodium acetate in 5.8 N acetic acid). All Si and Ge concentration assay were performed in duplicate. For assaying dry cell mass, a 90 mL. aliquot of cell suspension was centrifuged at 4,000 rpm (2,000 g) for 10 minutes. The supernatant was drawn off, the pellet was suspended in 45 mL de-ionized water and centrifuged again; this washing step was repeated two more times to completely remove soluble salts. The washed pellet was transferred to an aluminum weighing dish, weighed, oven dried in air at 80° C. for 24 hours, and then weighed after drying.

The silicon and germanium content in the cell mass was determined after fusion with molten NaOH. A 20 mg aliquot of dry biomass was weighed to precision of 0.1 mg and then added to 1.0 g solid NaOH in a zirconium crucible. The mixture was heated at 400° C. within a furnace in air for 25 minutes. After cooling, the NaOH-fused sample was dissolved in 20 mL de-ionized H₂O and then neutralized to pH 7.0 with 1.4 M HNO₃. The neutralized solution was spectrophotometrically assayed for soluble Si and Ge concentration as described above, and the amount of Si and Ge in the biomass was determined from these concentration values, the total volume of neutralized solution, and the original dry cell mass.

Intact silica frustules were prepared by hydrogen peroxide treatment. The procedure was designed to minimize frustule breakage due to drying and mixing of the sample. Specifically, a 100 mL aliquot of the culture suspension sampled from the bioreactor was allowed to settle in a 200 mL sealed glass bottle for 6 hours. About 90 mL of supernatant was removed with a pipette, and the dense cell slurry was re-suspended in 100 mL de-ionized water and then allowed to settle again. The supernatant was removed with a pipette, leaving behind about 10 mL of dense cell suspension in de-ionized H₂O. The cell suspension was transferred to a 50 mL centrifuge tube.

To initiate oxidation of organic materials in the cell mass, 30 mL of 30 wt % aqueous hydrogen peroxide (H₂O₂) was added to the tube, along with 0.2 mL of 37 wt % HCl to lower the pH to 2.5 for removal of carbonates. The oxidation reaction was unmixed at room temperature for at least 48 hours. During this time the cell mass color changed from yellow-green to white. The uncapped centrifuge tube and contents were degassed under vacuum at room temperature for 1.0 hour, which removed oxygen bubbles and allowed the hydrogen peroxide treated cell mass to settle. The degassed supernatant was pipetted from the white particle slurry. The slurry was re-suspended in 30 mL MeOH and allowed to settle. The supernatant was removed by pipette, and the MeOH washing procedure was repeated two more times to completely remove water. The frustules, white in color, were stored at 4° C. in 10 mL MeOH within a sealed glass vial.

A 5-10 mg aliquot of the frustule suspension in MeOH described above was dried in air. The solid mass was weighed to precision of 0.1 mg and then subjected to the NaOH fusion procedure described above. A 20 mL aliquot of the final solution was then assayed for Si, Ge, Ca, and Mg by Ion-Coupled Plasma (ICP) analysis using a Varian (Liberty 150) ICP emission spectrometer. The analysis wavelengths were 251.611 (Si), 265.118 nm (Ge), 279.553 nm (Mg) and 393.366 nm (Ca). Limits of detecting Si, Ge, Ca, and Mg in the assay solution were 0.07, 0.04, 0.01, 0.04 mg/L respectively.

Pinnularia sp. frustules isolated by hydrogen peroxide treatment were analyzed by scanning electron microscopy (SEM) using an FEI Sirion field emission SEM and by transmission electron microscopy (TEM) using an FEI Tecnai F20 high resolution TEM (200 keV) equipped with embedded scanning transmission electron microscopy (STEM) and an X-ray energy dispersive analysis (EDS) probe. For SEM analyses, about 20 μL of the frustule suspension in methanol (diluted 10:1 v/v) was pipetted onto a carbon tape affixed to an aluminum stub. The sample was not coated with a conductive carbon layer. To avoid surface charging, the FESEM was operated at a low acceleration voltage of 2 keV. For TEM and STEM-EDS analyses, about 20 μL of frustule suspension in methanol (diluted 10:1 v/v) was pipetted onto a Holey carbon copper TEM grid (Electron Microscopy Services HC300-Cu, 3.05 mm diameter, 300 mesh, 54 μm hole width, 31 μm bar width). As the methanol evaporated to dryness, about 2-5 frustules per square were randomly dispersed onto the TEM grid. EDS elemental line scans were acquired in STEM mode (200 keV, tilt angle of 15 degrees) with sampling increment dependent on the length of the line scan (24 sample points if line was <500 nm, 49 sample points if line was >500 nm, 5,000 msec per sample point). Characteristic Kα energy peaks (all keV) were identified as 0.52(O), 1.25(Mg), 1.74(Si), 2.62 (Cl), 3.69(Ca), 9.86(Ge, Kα₁), 9.88 (Ge,Kα₂).

The frustule sample was weighed (ca. 1.0 mg) and loaded into a 3.0 mm square, 1.0 mm deep Delron sample holder. Photoluminescence measurements were performed at room temperature using a N₂-laser excitation source at 337 nm wavelength and average power source of 2.0 mW. The emission spectra was filtered at 400 nm and then captured on an Inspectrum 300 CCD detector (Acton Research) with an integration time of 200 msec.

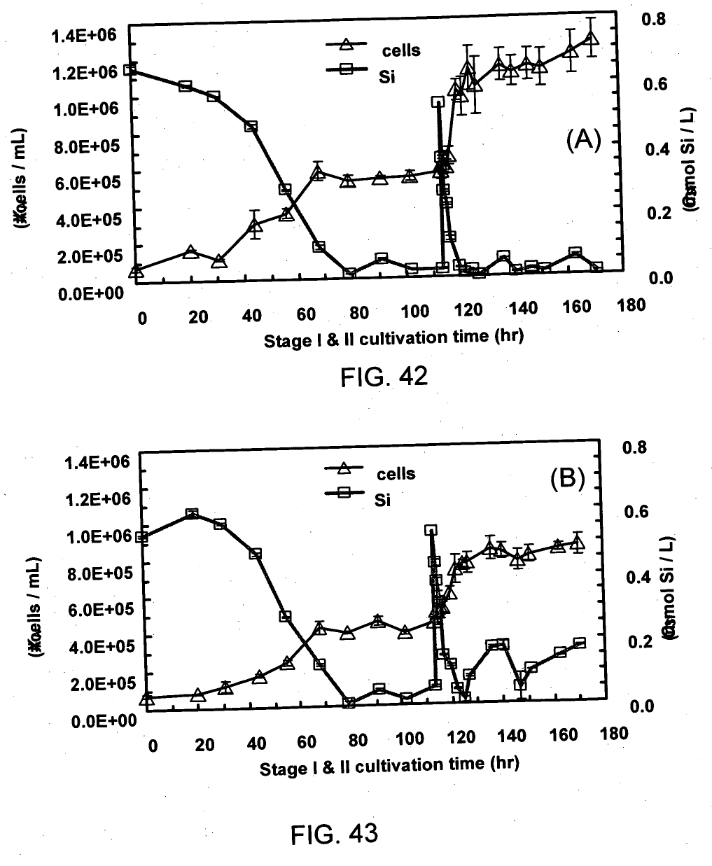

In Stage I, soluble silicon was added to the cell suspension culture, and the culture was grown up to silicon starvation. In Stage II, a mixture of soluble silicon and germanium was added to the silicon-starved culture. The cultivation conditions for Stages I and II are provided in Table 2, and the culture growth parameters in each stage for each cultivation are summarized in Table 3. Cell number density and dissolved silicon concentration in the culture medium versus cultivation time for Stages I and II for the control experiment where no germanium was added to Stage II is presented in FIG. 42. In Stage I, the amount of silicon added to the culture was designed to accomplish three cell doublings, so that at an initial cell number density of 7.5·10⁴ cells/mL, and final cell number density was nominally 6.0·10⁵ cells/mL. In Stage I of cultivation, silicon consumption was growth associated, and the cell number density increased proportionally to the decrease in dissolved silicon concentration with time. When all the dissolved silicon in the culture medium was consumed, the cell number density leveled off and then became constant with time. Silicon starvation was defined as a culture state where both the cell number density was constant and the dissolved silicon concentration was zero for at least one photoperiod (24 hours). In Stage I of cultivation, the pH rose from 8.3 at inoculation to 9.0 at the end of stationary phase. In Stage II of cultivation, soluble silicon was added to the silicon-starved culture at 4 hours into the 14 hour light portion of the 24 hours (14 hours light/10 hours dark) photoperiod, and the cultivation was allowed to proceed for an additional 56 hours. The amount of silicon added to the Stage II culture provided for one more cell number doubling. In Stage II, surge uptake of silicon was observed, where most of the dissolved silicon was taken up by the culture within the remaining 10 hours of the first photoperiod. After 12 hours, cell doubling was complete, the cell number density leveled off, and the culture was considered silicon starved once again. Cell number density (X_{N}) and dissolved silicon concentration (C_{Si}) in the culture medium versus cultivation time for Stages I and II is for the control experiment where 38 μmol/L of soluble germanium and 0.53 mmol/L soluble silicon were co-added to Stage II is presented in FIG. 43. After 38 μmol/L of soluble Ge was added to Stage II, the cultivation was similar to the control with no germanium.

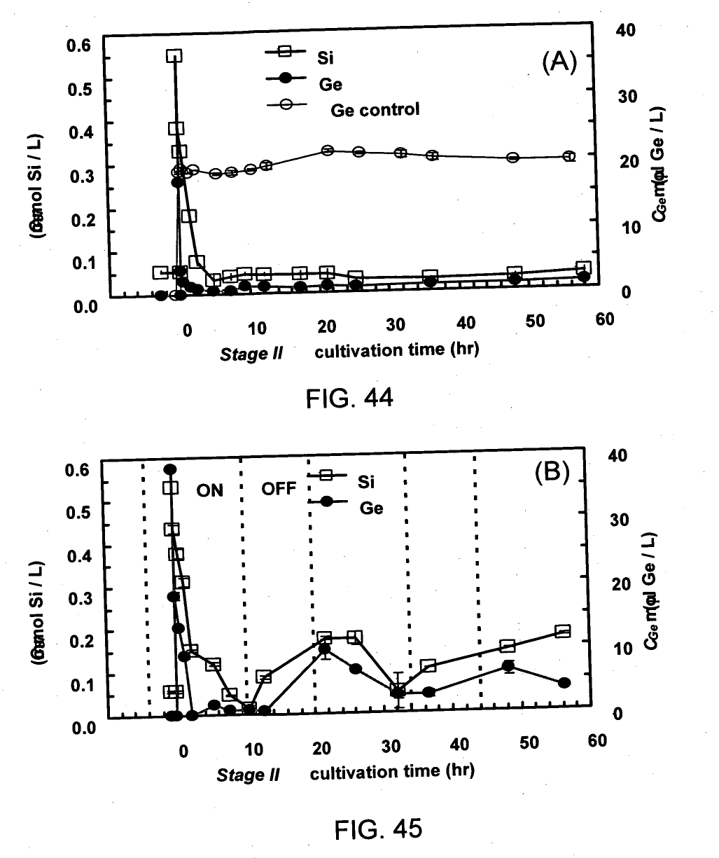

During Stage II of the cultivation process,.both soluble silicon and germanium were transported into the silicon-starved diatom cell by a first-order process during the illumination phase of the photoperiod, consistent with the mechanism of surge uptake during the G2 phase cell cycle. In fact, approximately two thirds of the silicon and germanium were taken up within 2 hours, although cell doubling was not complete until 12 hours into Stage II. Profiles of dissolved silicon and germanium concentration (C_{Ge}) versus cultivation time during Stage II at initial Ge concentrations of 17 and 38 μmolGe/L are presented in FIGS. 43 and 44 respectively. During the first 10 hours of Stage II, soluble Ge uptake by the diatom cell suspension culture was commensurate with the surge uptake of soluble Si. At the initial soluble germanium concentration of 17 μmol/L, the dissolved germanium and silicon concentrations in the medium remained at zero after the initial surge uptake. However, at the higher initial soluble germanium concentration of 38 μmol/L, the concentration of soluble Ge and Si did not stay at zero but instead cycled up and down in a pattern commensurate with the photoperiod. Specifically, when the lights when off, some Si and Ge were released back into the liquid medium. However, when the lights came back on, both the Si and Ge were taken up once again. Control experiments with no cells at an initial Ge concentration of 20 μmol/L confirmed that Ge did not adsorb onto the surfaces of the bioreactor (FIG. 44). As long as Ge uptake during Stage II was complete, the culture growth parameters—specific growth rate, cell number yield, and biomass silicon content—were nominally constant as the Stage II initial Ge concentration was increased (Table 3). However, at a Stage II initial Ge concentration of 38 μmol/L, partial Ge efflux was evident and was tied to the dark phase of photoperiod (FIG. 45), suggesting that Ge was ultimately inhibitory to the coupled machinery of frustule biosynthesis and cell division. From the bioprocessing perspective, germanium was simply an alternative substrate that was co-metabolized in the presence of silicon, the required substrate for cell division. Hence, for this disclosed embodiment, one feature of the disclosed bioprocessing strategy was the co-addition of soluble germanium with a sufficient amount of soluble silicon to support one cell division in Stage II (FIG. 43). During the single cell division in Stage II of the cultivation process, both Ge and Si were taken up into the cell (FIGS. 44, 45). When each cell divided, the new cell would contain a parent valve and a new valve of novel morphology containing germanium.

Results for the solid analysis of the bioreactor cultured cells are provided below. The silicon content in the dry cell mass at the end of Stage II for each photobioreactor experiment is presented in FIG. 46. The germanium content within the dry cell biomass at Stage II cultivation times of 12 hours and 56 hours are compared in FIG. 47 for Stage II initial Ge concentrations of 17 to 38 μmol/L. The silicon content in the dry cell mass was stable as the germanium content in the cell mass increased.

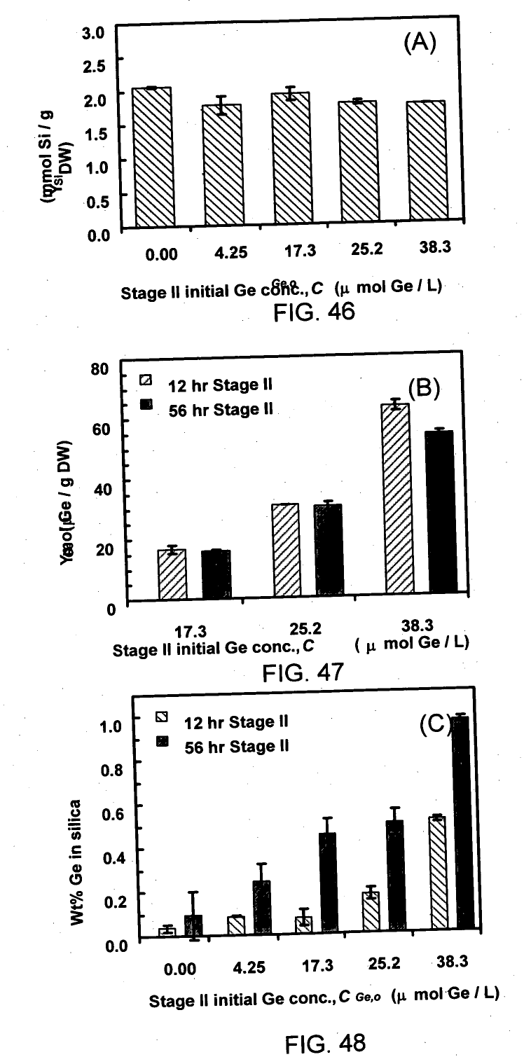

Results of the elemental (ICP) analysis of Ge in the silica frustules isolated by hydrogen peroxide treatment of the cell suspension obtained at two Stage II cultivation times of 12 and 56 hours are presented in FIG. 48 at Stage II initial germanium concentrations ranging from 0 (control) to 38 μmol/L. The hydrogen peroxide treatment was designed to gently oxidize organic materials away from the insoluble silica frustule. Only 14-15 wt % of the total dry cell mass remained insoluble after hydrogen peroxide treatment (Table 4), and this inorganic solid yield wasnot affected by the initial concentration of Ge in Stage II. Errors in the elemental analysis reflected the errors incurred during the hydrogen peroxide treatment step, not the ICP analysis. Residual calcium (Ca) and magnesium (Mg) remained in the frustule after hydrogen peroxide treatment, with compositions ranging 0.10 to 0.25 weight percent (Table 4). From material balance studies conducted on the hydrogen peroxide treatment step, only about 36±4.7% of the Ge initially in the cell mass sample still remained within the intact silica frustule; the rest of the Ge dissolved during hydrogen peroxide treatment and was recovered in the washings. The Ge contents of the frustules were very consistent, particularly for photobioreactor experiments conducted at Stage II initial Ge concentrations of 17-38 μmol/L, where the frustule germanium content ranged from 0.452±0.072 and 0.965±0.012 wt % after 56 hours of Stage II cultivation. A small amount of germanium was also detected by ICP in the control sample (initial concentration of 0.0 μmol/L Ge added to Stage II), but within the limits of detection error, the Ge content was zero. The Ge content in the frustule after 12 hours of Stage II cultivation was consistently about two times smaller than the germanium content after 56 hours. In contrast, the Ge content in the dry cell biomass before hydrogen peroxide treatment was nominally the same after 12 and 56 hours.

Scanning electron microscopy (SEM) of representative frustules obtained by hydrogen peroxide treatment of Pinnularia sp. cell biomass obtained from the end of the Stage I of the photobioreactor cultivation (114 hours) are presented in FIGS. 48 and 49. SEM images revealed the frustules were intact, and possessed levels of order spanning the micron and submicron (100-1000 nm) scales. The aqueous hydrogen peroxide treatment procedure did not always break apart the frustule into its upper epi-valve and lower hypo-valve; both are visible in FIG. 49.

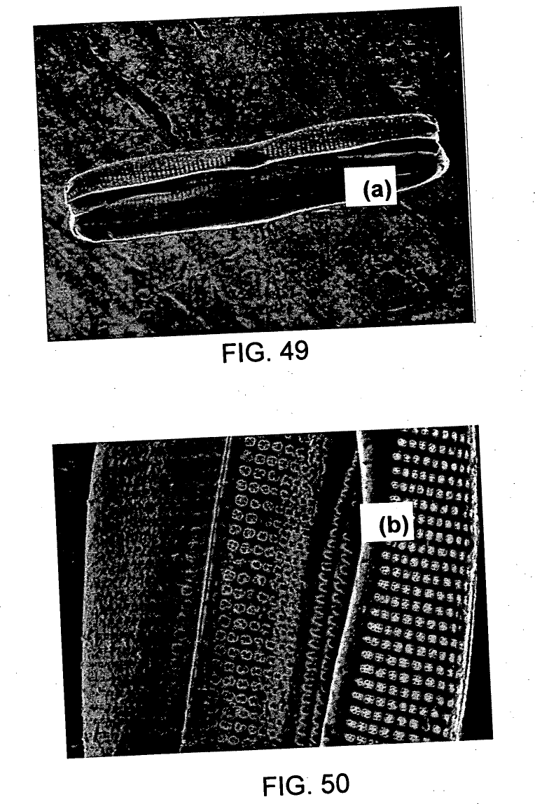

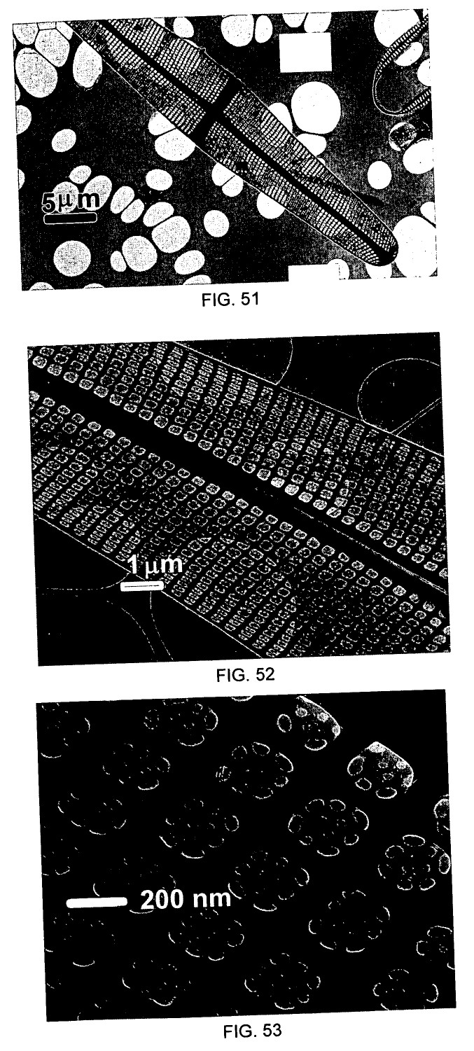

Frustules of the marine diatom Pinnularia sp. were prepared from hydrogen peroxide treatment of cell biomass generated by a two-stage bioreactor cultivation process described above. TEM images of a representative Pinnularia sp. frustule obtained from cell biomass cultivated on silicon with no germanium after 56 hours of Stage II cultivation are shown in FIGS. 51, 52, and 53 at micron, submicron (100-1,000 nm) and nanoscale (1-100 nm) respectively. A strip of thickened silica called a raphae ran axially down the length of the frustule (FIG. 51). Lateral ribs (costae) ran perpendicular to the raphae at 200 nm intervals (FIG. 52). The costae formed rows of ˜200 nm areolae, each of which possessed a “bottom” of thin silica containing a concentrically-arranged array of 5-10 smaller pores called velum (FIG. 53).

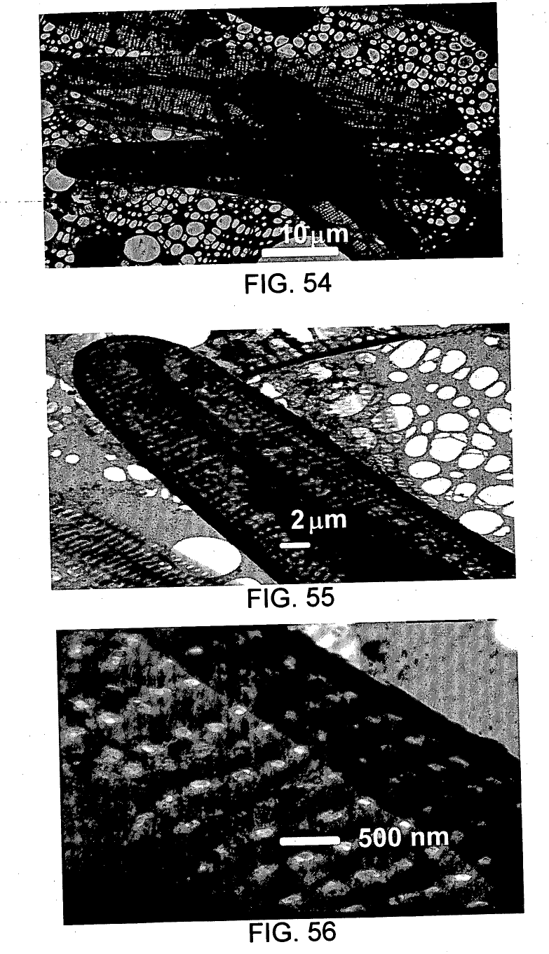

In subsequent experiments, mixtures of soluble silicon and germanium were added to the culture to initiate Stage II of the cultivation. TEM images of a representative Pinnularia sp. frustule obtained from hydrogen-peroxide treated cell mass containing 0.96 wt % germanium (56 hours of Stage II cultivation) are presented in FIGS. 54, 55, and 56 at the micron, submicron, and nanoscale respectively. EDS elemental analysis confirmed that the frustule itself now contained germanium, with a distinct Kα_{1,2} peaks for Ge at 9.86-9.89 keV. The incorporation of germanium into the biogenic silica altered frustule morphology. Although the overall shape of the frustule containing 0.96 wt % Ge was not changed (FIG. 54), the raphae thickened (FIG. 55). The lateral ribs (costae) also thickened and assumed a wavy shape. Although pore arrays were still observed, they were not always linear (FIG. 56). The areolae filled in with silica, and the velum partially fused together to form pores of 50 to 100 nm nominal diameter.

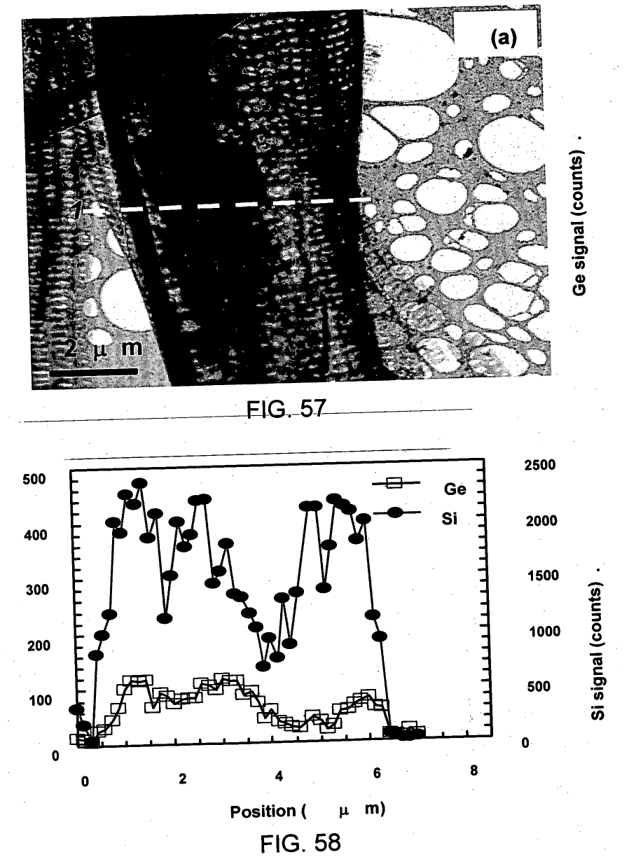

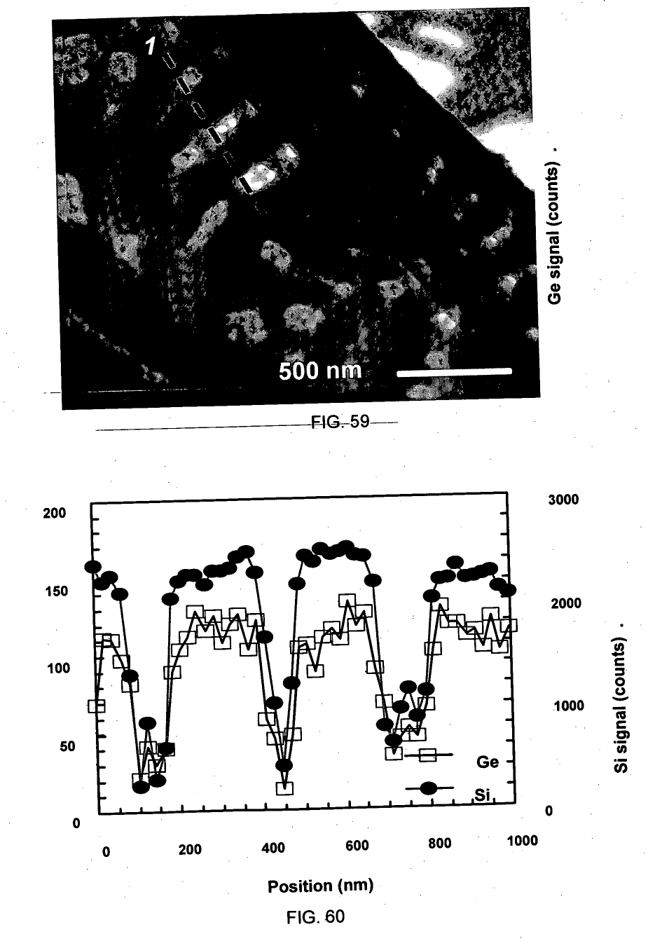

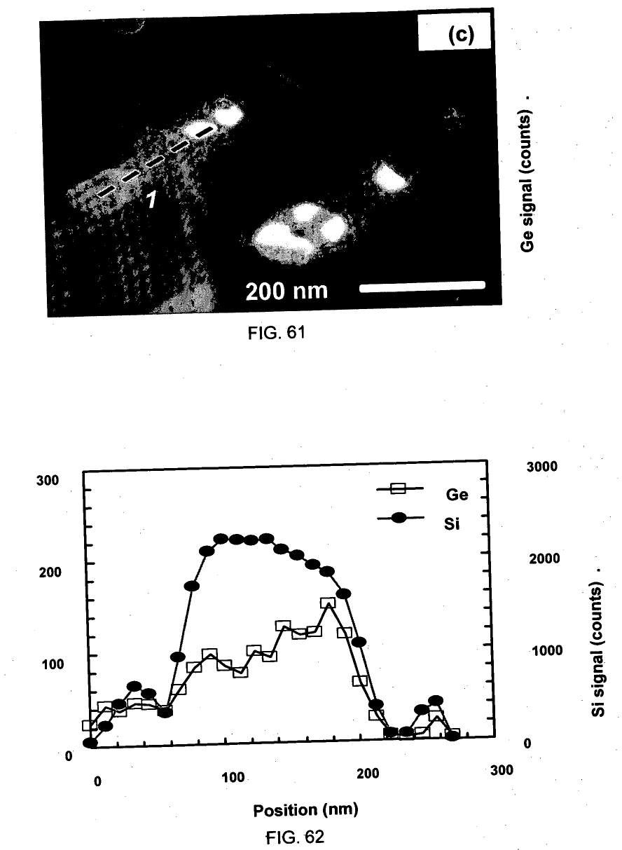

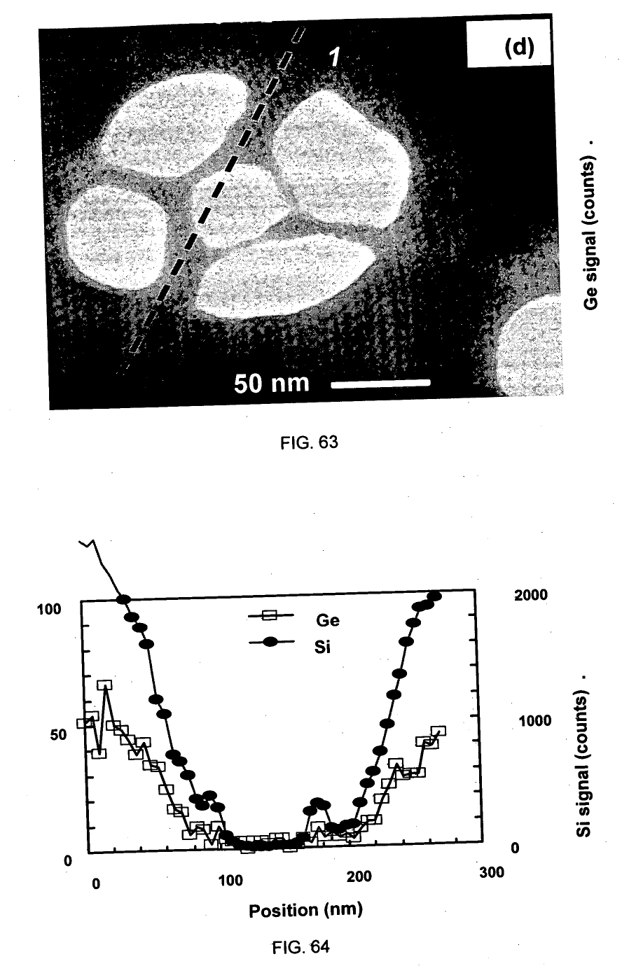

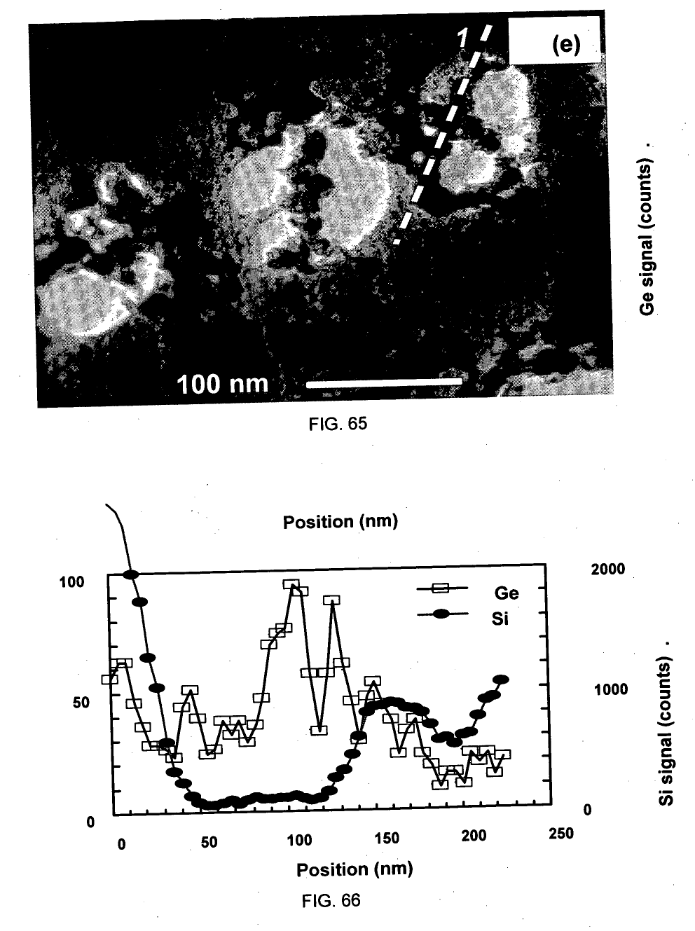

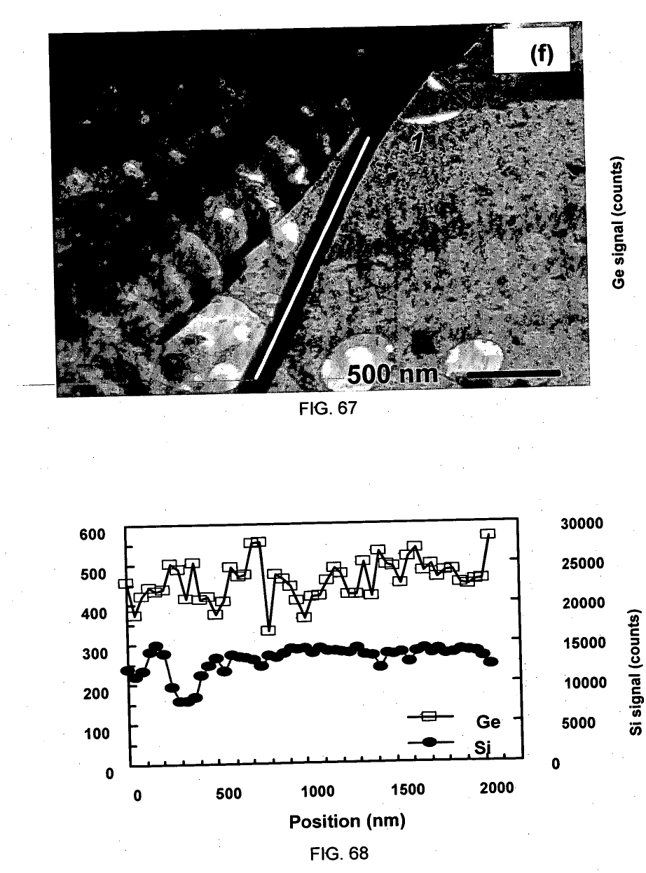

Results of the STEM-EDS analysis of the composite nanostructure after germanium addition are provided below. STEM-EDS line scans of silicon and germanium were performed on selected sub-micron features of the frustule in the attempt to identify Si—Ge nanostructures. An elemental line scan running across the entire frustule showed that germanium was present throughout the material (FIGS. 57-58). The Si and Ge line scans were jagged because the line scan periodically intercepted a pore space. Specific sub-micron features considered the solid regions across a series of lateral ribs (FIGS. 59-60), solid region between two pores (FIGS. 61-62), nanostructures within a single areolae (FIGS. 63-64), and a solid region containing nanoparticles partially filling an areolae (FIGS. 65-66). In general, germanium was dispersed into silica for features at both the sub-micron (100-1000 nm) and-nanometer (1-100 nm) scales. Furthermore, there were several “pockets” of solid material containing elevated levels of germanium or germanium gradients (FIGS. 61-62). Finally, solid regions with nanoparticle chains were significantly enriched in germanium (FIGS. 65-66). Putative girdle band structures were also enriched in germanium (FIGS. 67-68).

In summary, metabolic insertion of Ge was used to controllably reduce the pore aperture diameter and elongate the pore shape, perhaps into a slit-like geometry. Controlled alteration of nanoscale frustule pore aperture arrays by metabolic insertion of Ge has not been previously reported in diatoms. Metabolic insertion of germanium into the biogenic silica selectively altered frustule morphology. The overall shape of the diatom was intact at the micron scale. However, at the submicron (100-1000 nm) and nanoscales (1-100 nm), the alloyed Si and Ge oxides resulting from metabolic insertion of Ge essentially thickened and at the same time “spread out” the biogenic silica, which in turn filled in or distorted the geometry of the pores. In addition to alterations in frustule morphology, there were germanium-rich pockets imbedded in the silica frustule and germanium rich nanoparticles randomly littering the frustule surface.

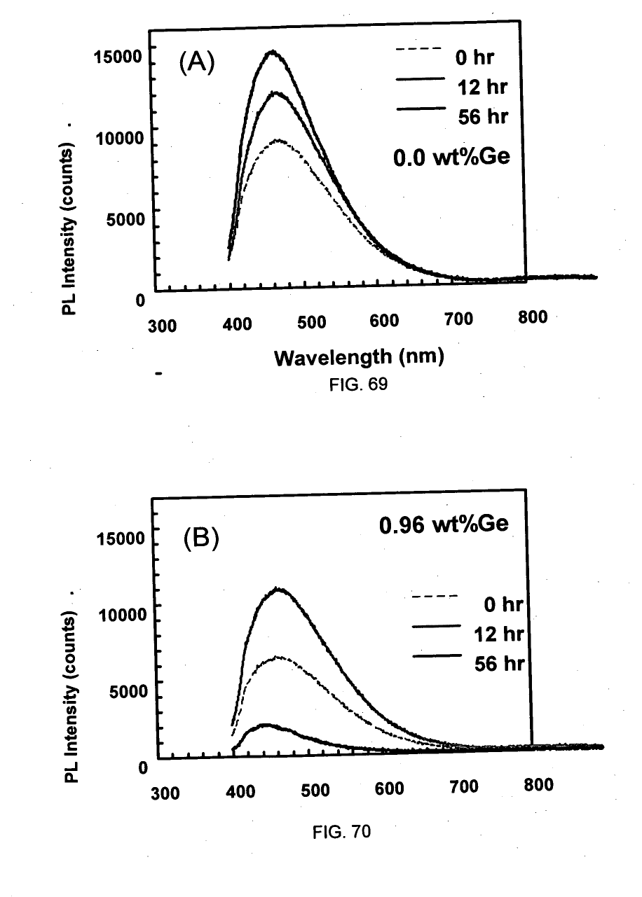

Results from measurement of photoluminescence of Pinnularia sp. frustules are provided below. Pinnularia sp. frustules containing no germanium and 0.96 wt % germanium both possessed strong blue photoluminescence (FIGS. 69, 70). Without being limited to a theory of operation, it appears that the strength of the photoluminescent signal was dependent upon cultivation time in Stage II and on the final germanium content after hydrogen peroxide treatment of the cell mass. The results showed that the metabolic insertion of germanium into the frustule caused a blue shift in the peak wavelength of the spectrum (FIG. 70) and at the same time controllably attenuated the signal strength (FIG. 70). This result clearly shows that metabolic insertion of germanium into the frustule alters the optoelectronic properties of the material.

TABLE 2

| | | Stage I | Stage II |
|---|---|---|---|
| Process Parameter | | | |
| Target Initial | | | |
| conditions | | | |
| Cell number density | X_{N, o} | 7.5 · 10⁴ | 6.0 · 10⁵ cells/mL |
| | | cells/mL | |
| Initial silicon | C_{Si, o} | 0.53 mmol Si/L | 0.53 mmol Si/L |
| concentration | | | |
| Initial germanium | C_{Ge, o} | 0.0 mmol Ge/L | 0.0 μmol Ge/L (control) |
| concentration | | | 4.25-38.3 μmol Ge/L |
| | C_{Si, o}/ | | 126-13.9 (mole basis) |
| | C_{Ge, o} | | 49-5.4 (mass basis) |
| Process Conditions | | | |
| Temperature | T | 22° C. | 22° C. |
| Initial culture | Vₒ | 3.90 L | 3.04 L |
| volume | | | |
| Incident light | Iₒ | 149 ± 15 | 149 ± 15 μE/m²-sec |
| intensity | | μE/m²-sec | |
| Fractional | f | 0.583 | 0.583 |
| photoperiod | | (14 hours light/ | (14 hours light/10 |
| | | 10 hours dark) | hours dark) |
| Aeration rate | v_{g} | 0.65 L air L⁻¹ | 0.83 L air L⁻¹ min⁻¹ |
| | | min⁻¹ | |
| CO₂ partial | P_{CO}_{₂} | 350 ppm | 350 ppm (ambient air) |
| pressure | | (ambient air) | |
| Cultivation time | t | 114 hours | 56 hours |

TABLE 3

| | | Initial Germanium Concentration in Stage II | | | | |
|---|---|---|---|---|---|---|
| rowth Parameter | Stage | 0.0 μmol/L | 4.25 μmol/L | 17.34 μmol/L | 25.2 μmol/L | 38.3 μmol/L |
| Specific growth rate | I | 0.0515 ± 0.0084 | 0.0489 ± 0.0019 | 0.0446 ± 0.0034 | 0.0493 ± 0.0050 | 0.0572 ± 0.0050 |
| μ^{/}(hr⁻¹) | II | 0.0656 ± 0.0079 | 0.0605 ± 0.0100 | 0.0368 ± 0.0033 | 0.0370 ± 0.0045 | 0.0447 ± 0.0041 |
| Cell number yield | I | 7.23E+08 ± 1.05E+08 | 6.74E+08 ± 1.44E+08 | 9.17E+08 ± 5.75E+07 | 5.93E+08 ± 1.31E+08 | 6.60E+08 ± 9.41E+07 |
| Y_{Xn/Si} (#cells/ | II | 1.16E+09 ± 1.28E+08 | 1.12E+09 ± 8.90E+07 | 8.37E+08 ± 7.32E+07 | 9.54E+08 ± 9.74E+07 | 9.16E+08 ± 5.26E+07 |
| mmol Si) | | | | | | |
| Dry cell mass yield | I | 0.324 ± 0.0034 | 0.432 ± 0.0028 | 0.000 ± 0.0000 | 0.347 ± 0.0051 | 0.335 ± 0.0033 |
| Y_{X/Si} (g DW/mmol Si) | II | 0.777 ± 0.064 | 0.803 ± 0.090 | 0.000 ± 0.000 | 0.790 ± 0.075 | 0.465 ± 0.044 |
| Final cell mass density | I | 0.254 ± 0.0014 | 0.325 ± 0.0014 | 0.000 ± 0.0000 | 0.295 ± 0.0042 | 0.195 ± 0.0014 |
| X_{f} (g DW/L) | II | 0.653 ± 0.054 | 0.741 ± 0.083 | 0.000 ± 0.000 | 0.696 ± 0.065 | 0.383 ± 0.036 |
| Final cell number | I | 5.42E+05 ± 2.04E+04 | 5.47E+05 ± 2.25E+04 | 6.66E+05 ± 3.24E+04 | 5.30E+05 ± 3.29E+04 | 4.13E+05 ± 3.44E+04 |
| density | II | 1.17E+06 ± 9.55E+04 | 1.16E+06 ± 8.78E+04 | 1.10E+06 ± 6.63E+04 | 1.09E+06 ± 9.96E+04 | 8.15E+05 ± 4.50E+04 |
| X_{N, f} (#cells/mL) | | | | | | |
| Mean light intensity | I | 118 | 118 | 113 | 118 | 123 |
| Iₘ (X_{N, f}) | II | 97 | 97 | 99 | 99 | 108 |
| (μE/m²-sec) | | | | | | |
| Peak CO₂ demand | I | 0.324 | 0.382 | 0.000 | 0.212 | 0.170 |
| Qₒ (X_{f}) (mmol | II | 0.833 | 0.872 | 0.000 | 0.501 | 0.333 |
| CO₂/L-hr) | | | | | | |
| Initial Si uptake | II | 0.600 ± 0.176 | 0.726 ± 0.116 | 0.771 ± 0.122 | 0.715 ± 0.100 | 0.597 ± 0.066 |
| rate k^{/}_{Si} (mL/10⁶ | | | | | | |
| cells-hr) | | | | | | |
| Initial Ge uptake | II | | | 1.118 ± 0.200 | 1.717 ± 0.405 | 1.256 ± 0.145 |
| rate k^{/}_{Ge} (mL/10⁶ | | | | | | |
| cells-hr) | | | | | | |

TABLE 4

| Stage II | | Initial Germanium Concentration in Stage II | | | | |
|---|---|---|---|---|---|---|
| Time (hr) | | 0.0 μmol/L | 4.25 μmol/L | 17.3 μmol/L | 25.2 μmol/L | 38.3 μmol/L |
| 12 [a] | Ge (wt %) | 0.036 ± 0.017 | 0.085 ± 0.0015 | 0.075 ± 0.0038 | 0.178 ± 0.027 | 0.511 ± 0.012 |
| | Ca (wt %) | 0.169 ± 0.084 | 0.036 ± 0.044 | 0.230 ± 0.176 | 0.009 ± 0.022 | 0.032 ± 0.021 |
| | Mg (wt %) | 0.132 ± 0.034 | 0.167 ± 0.027 | 0.213 ± 0.105 | 0.177 ± 0.020 | 0.160 ± 0.013 |
| 56 [a] | Ge (wt %) | 0.092 ± 0.110 [b] | 0.243 ± 0.081 | 0.452 ± 0.072 | 0.498 ± 0.061 | 0.965 ± 0.012 |
| | Ca (wt %) | 0.247 ± 0.149 | 0.140 ± 0.026 | 0.053 ± 0.038 | 0.086 ± 0.033 | 0.092 ± 0.131 |
| | Mg (wt %) | 0.142 ± 0.036 | 0.134 ± 0.022 | 0.111 ± 0.022 | 0.108 ± 0.032 | 0.106 ± 0.017 |
| 0-56 [c] | g H₂O₂ | 0.156 ± 0.046 | 0.158 ± 0.030 | | 0.144 ± 0.023 | 0.159 ± 0.060 |
| | solid/gDW | | | | | |

Notes
[a] ±1.0 S.D for n = 3 samples (3× replicates per sample)
[b] Analyses near ICP detection limit for Ge
[c] Average (±1.0 S.D.) from Stage II cultivation times of 0, 12, 36, and 56 hours (n = 4 with 2 replicates per sample)

### Example 12

This example concerns the fabrication and testing of an electroluminescent (EL) device using diatom frustules. A schematic of the electroluminescent device is provided in FIG. 30. To fabricate the device, a mixture of diatom frustules was dispersed in isopropanol and then spin-coated on a transparent indium tin oxide (ITO) glass plate of 300 nm (0.3 micron) thickness. After spin coating, the isopropanol was evaporated at room temperature, leaving behind a thin layer of diatoms on the ITO plate. A 100 nm thick hafnium oxide thin film was deposited over the diatom layer by atomic layer deposition (ALD). Finally, a 100 nm thick aluminum layer was deposited over the hafnium oxide layer by physical vapor deposition (PVD). In this present example, Pinnularia sp. diatom frustules containing 0.96 wt % germanium prepared according to Example 11 were used.

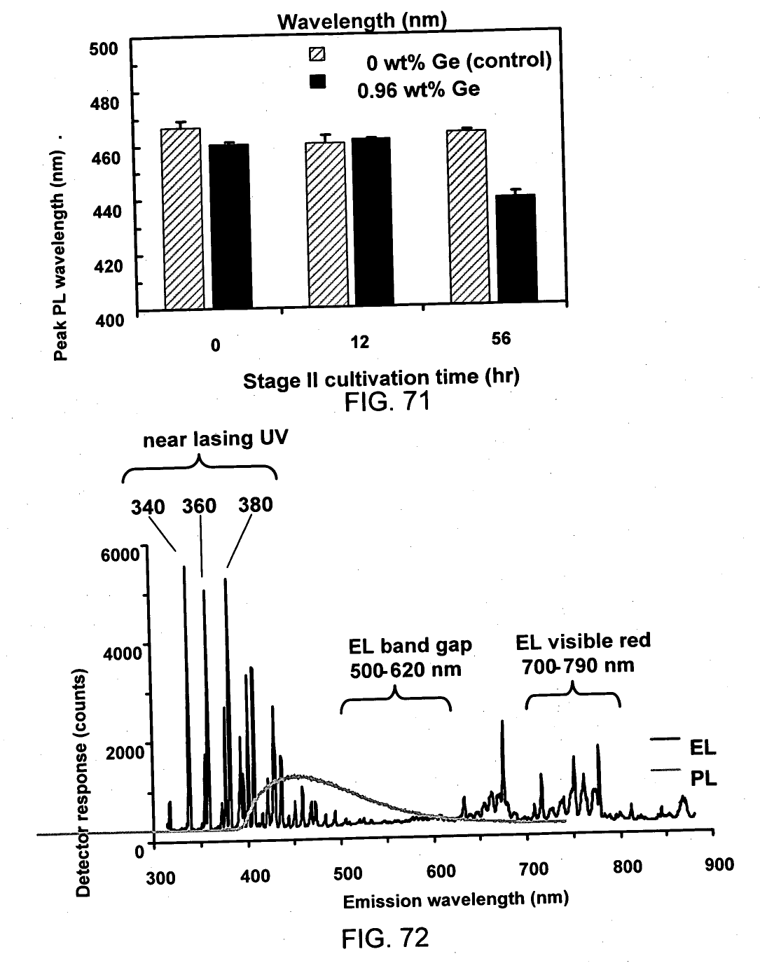

The ITO layer and the aluminum layer serve as electrodes for the EL device. The hafnium oxide layer serves as a dielectric coating over the diatom layer to increase the capacitance of the device. When a 200 V bipolar square pulse was passed across the electrodes, the diatom layer emitted the electroluminescence (EL) spectrum shown in FIG. 72. This EL spectrum possesses three sharp UV near-lasing emission lines at 340, 360, and 380 nm; an EL band gap between 500-620 run, and a visible red luminescence at 700-780 nm. For comparison, the photoluminescence (PL) spectrum of the same sample is provided in FIG. 41. The PL spectrum is distinctly different than the EL spectrum, which further points to the novelty of this device.

### Example 13

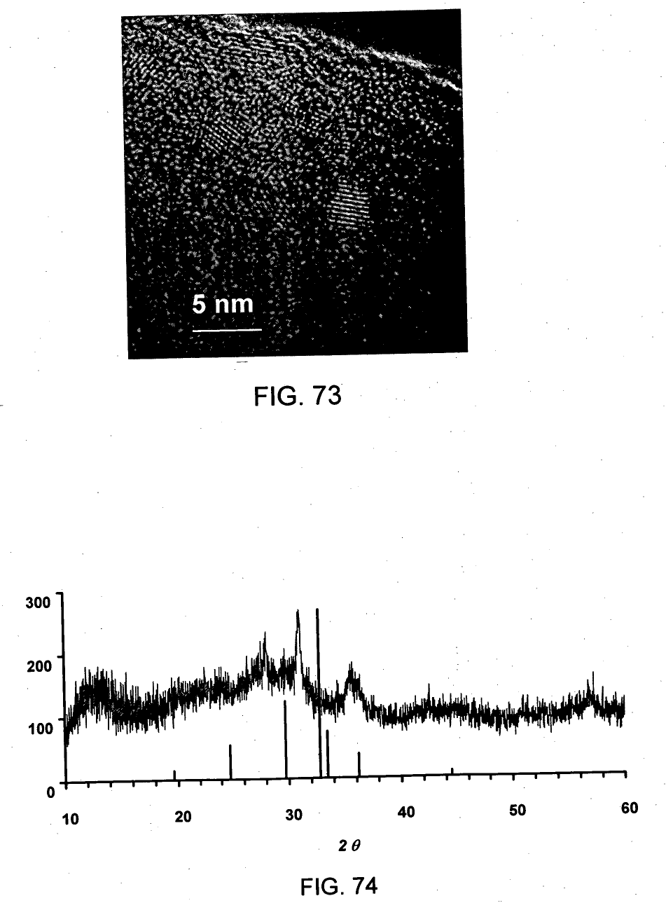

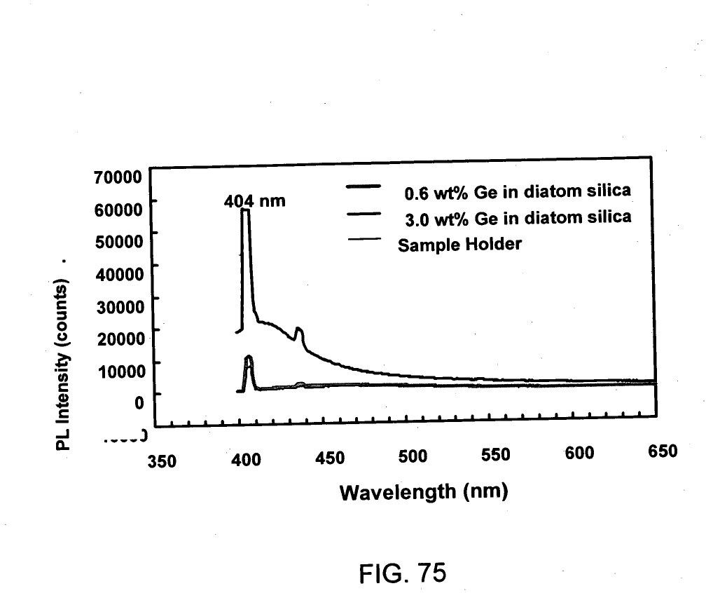

This example concerns the preparation of metal oxides that contain nanocrystalline Ge imbedded within an intact, amorphous silica frustule. In one embodiment of this method, pennate Nitzschia frustulum diatoms are cultivated in the presence of germanium as described in Examples 1 and 11, and then post processed by the aqueous hydrogen peroxide treatment method described in Example 11 to produce a mixed primary and secondary metal oxide that possesses nanostructured germanium oxides imbedded within the intact silica microstructure of the diatom frustule. The metal oxide material produced by these methods was then thermally annealed with a flowing gas mixture containing 5% hydrogen gas and 95% nitrogen gas at 1,000° C. and 1.0 atm for 3 hours. Hydrogen gas at these conditions reduces the germanium oxides to nanocrystalline Ge. However, this temperature was was high enough to reduce amorphous silica to silicon or to convert amorphous silica to crystalline silica. The TEM micrograph presented by FIG. 73 clearly shows the presence of ˜5 nm Ge crystals imbedded within silica. The X-ray diffraction analysis on the bulk solid confirms the presence of crystalline Ge (FIG. 74). Furthermore, due to the presence of Ge nanocrystals, at a bulk germanium content of 3.0 wt % in the diatom biosilica, this material had intense violet luminescence under UV lamp excitation at 254 nm, as shown in FIG. 75. The shape of the peak was sharp, and the intensity of the photoluminescence increased as the Ge content in the bulk solid increased.

Although Example 13 specifically concerns pennate Nitzschia frustulum diatoms, the processing information provided applies equally well to other types of diatoms including, for example, other Pennate diatoms such as Pinnularia sp. or Centric diatoms such as Cyclotella.

### Example 14

This example concerns the preparation of a nanocrystalline thin film of zinc silicate (Zn₂SiO₄) onto the frustule of the diatom Pinnularia sp. The biogenic frustule of Pinnularia sp. as prepared by Example 11 served as the source of silica for the synthesis of zinc silicate. A thin film of zinc silicate doped with manganese was fabricated on the surface of the Pinnularia sp. frustule by chemical bath deposition (CBD). First, 0.00063 mol of SiO₂ Pinnularia sp. frustule), 0.0056 mol of ZnCl₂ (Fisher Scientific, Technical grade) and 0.000015 mol of MnCl₂ (Fisher Scientific, ACS grade) were mixed in 10 ml DI water within a beaker. The beaker top was sealed, and the solution was then heated to 150° C. The mixture was hydro-thermally treated at this temperature for 1.0 hour. After chemical bath deposition, the solution was dried and resulting white powder was collected. The white powder was placed in a crucible and annealed in air within a furnace at 900° C. for 1.0 hour. The annealed white powder was suspended in methanol and pippeted onto a copper TEM grid coated with Lacey carbon film. The TEM sample was analyzed by TEM at 200 KeV using a Tecnai F20 field emission TEM with an embedded scanning transmission electron microscope (STEM) and energy-dispersive X-ray spectrophotometer (EDS). X-ray diffraction (XRD, Siemens D-5000 with Cu Kα radiation at a wavelength of 1.5418 Å) analysis was employed to identify the phases for the obtained powder. The as-deposited white powder obtained after the hydrothermal reaction step was placed in a pre-tarred aluminum pan. The temperature ramp was 5° C. per minute up to 600° C.

The photoluminescent spectrum was measured at room temperature with a 254 nm excitation source using a PL measurement system from ORIEL Corp. The excitation light source was a mercury UV-lamp with a monochrometer. The optical images of intact pinnularia frustule were taken using a Leica DMIL Microscope from Leica Microsystems Wetzlar GmbH. A mercury UV lamp with a 254 nm line was used to produce the green luminescent individual frustule image.

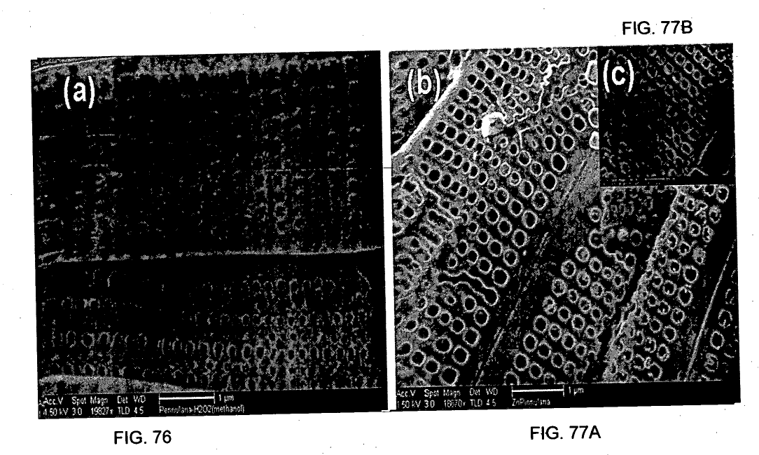

FIG. 75 shows an SEM image of Pinnularia sp. frustule isolated by aqueous hydrogen peroxide treatment of the bioreactor-cultured diatom cells. The frustule has a well-aligned, nano-sized pore structure (˜100 nm). The biogenic SiO₂ frustule was then 20 subjected to a chemical bath deposition process followed by a thermal annealing. FIGS. 76A, 76B show the SEM images of the frustule after the chemical bath deposition and annealing process. The submicron features of the frustule are still clearly observed, and the surface morphology remains smooth.

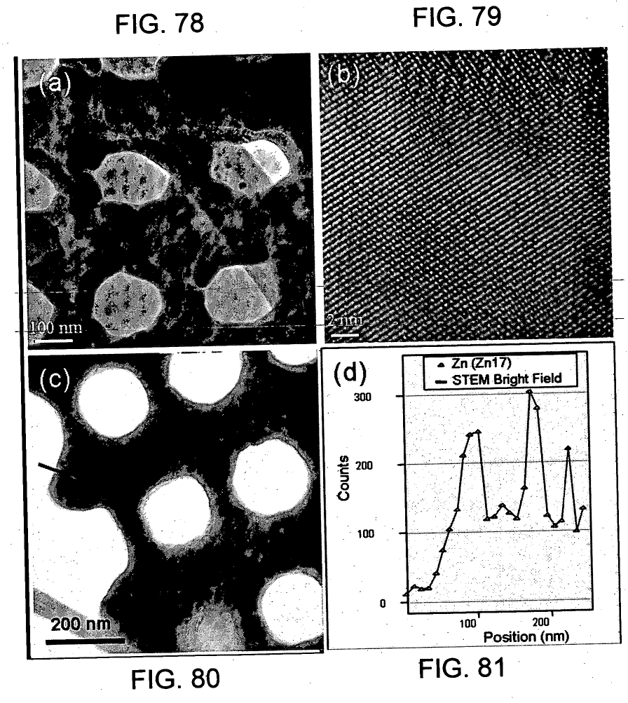

High resolution TEM images of the Pinnularia sp. frustule pore structure after chemical bath deposition and thermal annealing are presented in FIGS. 77-80. Two types of nanostructures could be seen. The first type of nanostructure is a nanocrystalline thin film (FIG. 77) with darker regions possessing a highly-ordered lattice (FIG. 78). The second type of nanostructure consists of dark nanospheres on the order ˜10 nm embedded in the lighter nanostructured silica (FIG. 79). An EDX line scan for Zn through this material is shown in FIG. 80. The peaks and valleys in the Zn line scan represent zinc distribution in between the dark and light regions. The EDX data indicate that the darker region contains a richer amount of zinc. Therefore, the second type of nanostructure consists of zinc-silicate nanospheres imbedded in amorphous nanostructured silica.

Formation of these different nanostructures may depend on the coverage of the deposits from the chemical bath deposition. The first type of nanostructure was formed by reacting a continuous ZnO thin film with the underlying armophous SiO₂ of the Pinnularia sp. frustule to form nanocrystalline Zn₂SiO₄:Mn. The second type of nanostructure was generated from discrete nanoparticles grown heterogeneously on top of the frustule silica surface before they coalescenced into a continuous film. These nanoparticle deposits then reacted with the underlying silica to form Zn₂SiO₄:Mn nanospheres embedded in the structure of the silica frustule during the high temperature thermal annealing (900° C.) process. Zinc silicate (Zn₂SiO₄) crystallizes in phenacite structure (space group R3) as both Zn²⁺ and Si⁴⁺ ions are coordinated tetrahedrally to four oxygen atoms. There are two non-equivalent crystallographic Zn sites accessible to Mn²⁺ ions. X-ray and electron diffraction were performed to determine the crystal structure of zinc silicate (FIG. 81). The powder x-ray diffraction patterns matched with the reference XRD diffraction patterns of zinc-silicate (JCPDS-37-1485) with a rhombohedral structure.

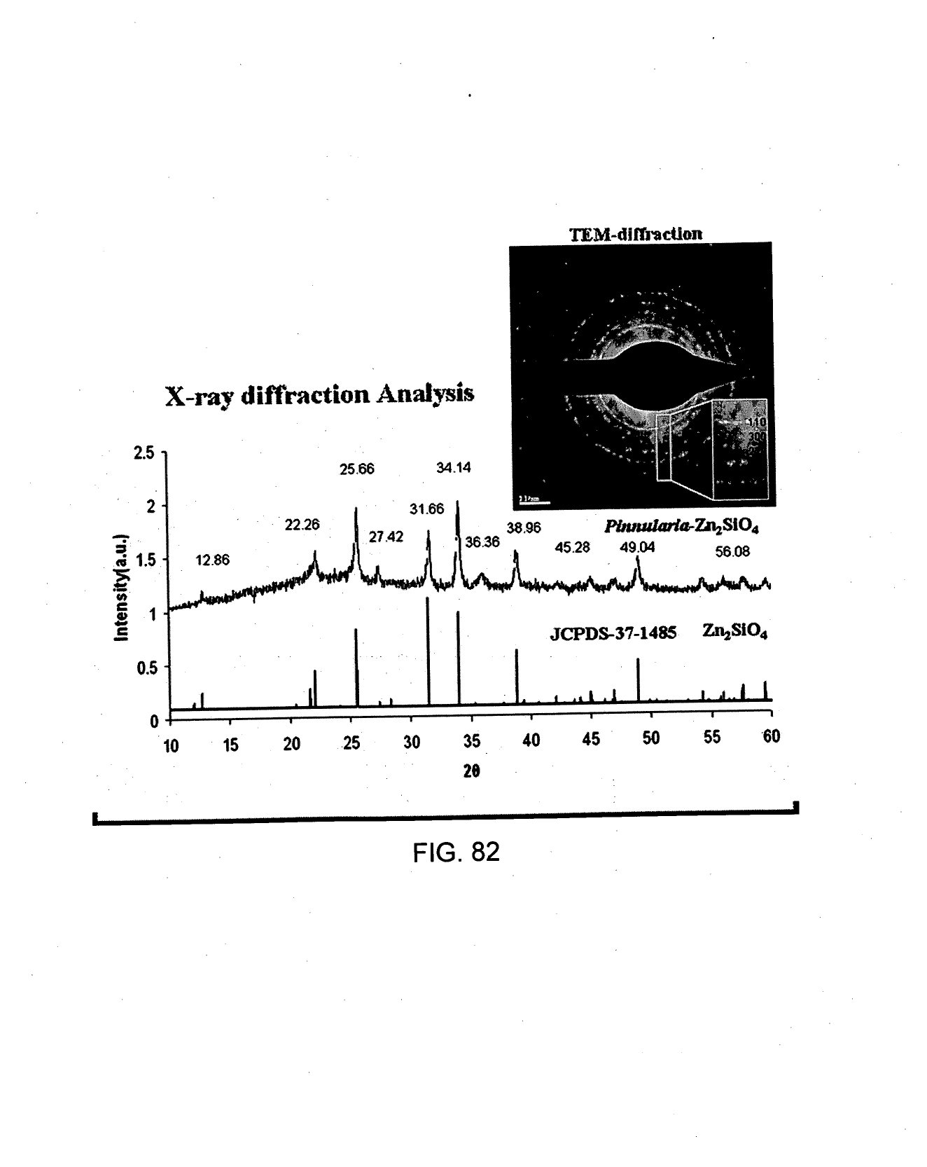

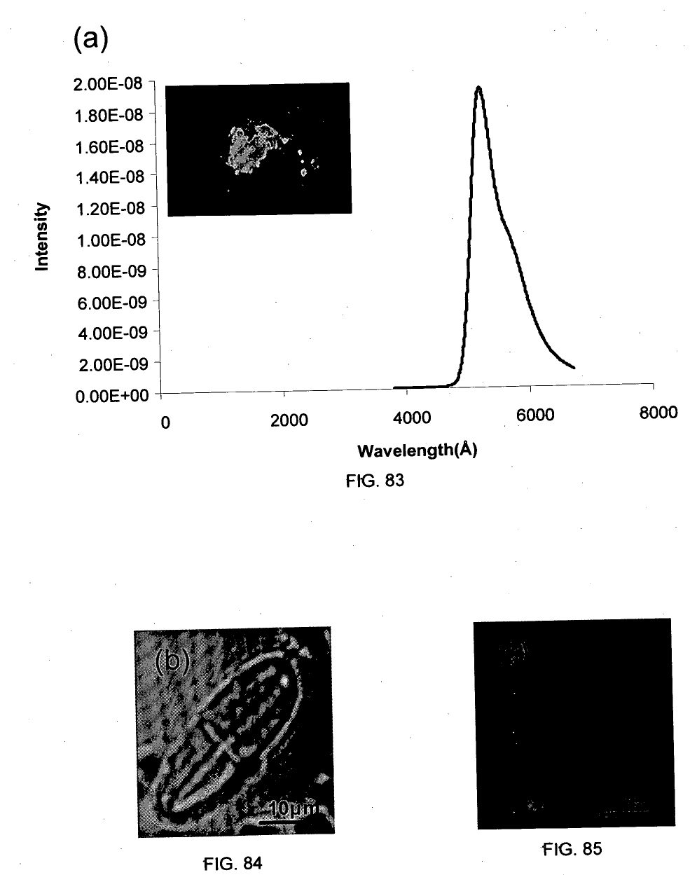

The luminescent properties of the samples were characterized by photoluminescence (PL) spectroscopy. The samples were excited by a monochromatic 254 nm light from a mercury UV-lamp. The PL spectrum intensity peaks at around 528 nm, which corresponds to green light (FIG. 82). There is, however, a shoulder peak closer to the yellow light wavelengths also present in the spectrum. The samples emit bright green luminescence visible to the naked eye (see FIG. 82 inset). FIGS. 83 and 84 are optical images of a Zn₂SiO₄:Mn coated diatom showing the intact features of the frustule under white light and UV light illumination, respectively.

The phosphor Zn₂SiO₄:Mn is useful for display applications due to its high efficiency and relatively long persistence. It is generally accepted that the green emission band is attributed to the spin flip transition of the d-orbital electron of the Mn²⁺ ion by the ⁴T_{1g}(⁴G)-⁶A_{1g}(⁶S) transition. It is also known that the luminescent emission spectra of Mn²⁺ ions depend strongly on the crystal field around the Mn²⁺ ions. For example, green, yellow, yellowish-white, and red emissions can be obtained from Zn₂SiO₄:Mn thin film electroluminescent devices depending on the processing condition. Thus the green and yellow-green emissions could both be attributed to the ⁴T_{1g}(⁴G)-⁶A_{1g}(⁶S) transition in Mn²⁺ ions.

### Example 15

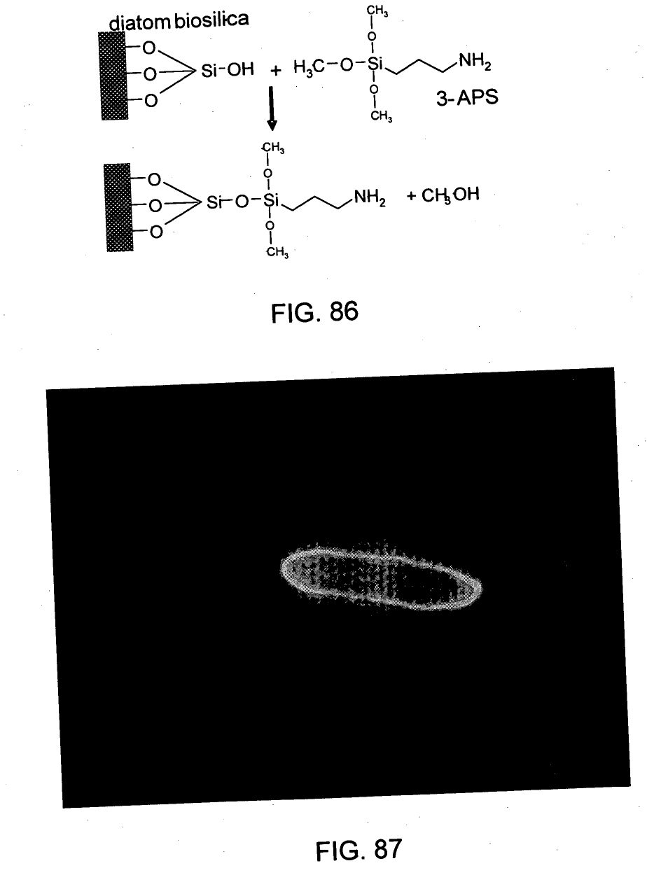

This example concerns the surface functionalization of diatom biosilica with amine groups. These amine groups provide a linker for attaching desired groups or compounds, such as bioactive molecules, to the surface of the intact frustule. In one embodiment of the method, intact Pinnularia sp. frustules prepared according to Example 11 are functionalized with amine groups using the reagent 3-APS (3-aminopropyl-trimethoxysilane). The chemistry of amine functionalization with diatom biosilica with 3-APS is shown in FIG. 86. Based on FTIR analysis of Pinnularia sp. frustules, diatom biosilica already has many Si—OH sites for coupling with 3-APS. Therefore, conversion of a portion of surface SiO₂ sites to Si—Oh sites is not required for diatom biosilica. A person of ordinary skill in the art will appreciate that the amine functional group is but one example of a functional group that can be coupled to diatom biosilica using the approach of this Example 15. Solely by way of example, other nitrogen-based functional groups also can be useful, such as hydrazides. The present invention is directed to all such functional groups.

A suspension of Pinnularia sp. frustules was washed in ethanol four times to prepare the frustules for amine functionalization. A 1.0 mL aliquot of this sample containing 0.5 mg frustules per mL of ethanol was transferred into an amber glass vial containing 1.0 mL of 3-aminopropyl trimethoxysilane (APS). This solution was heated to a low boil for 2 hours under continuous stirring followed by mixing on a rotary platform for 24 hours.

To verify that the amine groups were present on the diatom biosilica frustule, the amine groups were labeled with Fluorsecamine, a dye that only becomes fluorescent after it has reacted with the amine groups. To prepare the derivatized Fluorescamine probe, a 100 μL aliquot of the 0.5 mg/mL amine-functionalized frustule suspension was added to 300 μL of a 1 mg/mL fluorescamine solution in methanol in a black 1.5 mL microcentrifuge tube and then mixed under vortexing for 3 minutes. The derivatized Fluoroescamine probe, when excited at a wavelength of 390 nm, has an emission wavelength at 475 nm. Tagging the amine groups via fluorescamine derivatization allowed the amine coupling sites to be visualized directly on the frustule, as shown in the fluorescence microscope image given in FIG. 87 using a DAP 425 nm long-pass filter excitation source.

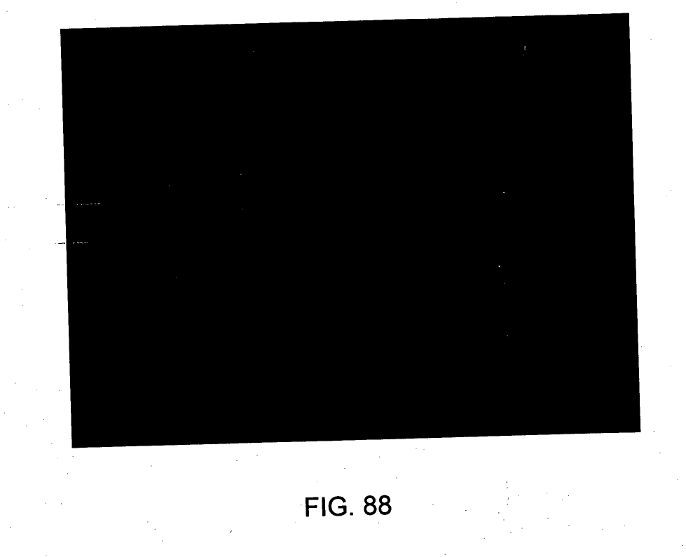

The same methods described above were used to functionalize the Centric diatom Cyclotella sp., as shown in FIG. 88. Therefore the method applies to all Pennate and Centric diatoms. Consequently, the functionalization pattern can be altered by simply applying this method to other diatoms, as diatoms species exhibit a broad spectrum of overall sizes and shapes and well as myriad of pore structures at the nanoscale and microscale.

Once the surface of the intact diatom frustule is functionalized with amine groups, standard amine coupling chemistry methods can be used to covalently attach N-terminal biomolecules such as peptides, proteins, streptavidin-biotin, antibodies, or any combinations thereof, to the amine-functionalized diatom frustule. Furthermore, any metal or metal nanoparticle which complexes with amine groups also could be attached to the frustule.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method for making a metal oxide, comprising:
selecting a substrate for making the metal oxide;
contacting a biological system with the substrate material for a period of time effective for the system to assimilate the substrate material; and
isolating the metal oxide from the biological system.

2. The method according to claim 1 where the metal oxide is a mixed metal oxide, a mixed semimetal oxide, or a mixed metal-semimetal oxide.

3. The method according to claim 1 where the metal oxide is a Si—Ge oxide.

4. The method according to claim 1 where the substrate is a metal oxide.

5. The method according to claim 1 where the substrate is a Group IVa or IVb metal oxide.

6. The method according to claim 1 where the substrate is selected from the group consisting of titanium oxides, zirconium oxides, hafnium oxides, silicon oxides, gallium oxides, germanium oxides, tin oxides, lead oxides, boron oxides, gallium oxides, indium oxides, and vanadium oxides, materials that produce such metal oxides, and all combinations thereof.

7. The method according to claim 5 where the substrate provides a soluble source of germanium.

8. The method according to claim 1 further comprising first cultivating the biological system in the presence of a soluble source of silicon to substantial silicon starvation.

9. The method according to claim 1 comprising coadministering germanic acid and silicic acid.

10. The method according to claim 1, further comprising:
cultivating the biological system in the presence of a soluble source of silicon to substantial silicon starvation; and
cultivating the biological system after substantial silicon starvation in the presence of a soluble source of germanium.

11. The method according to claim 1 where the biological system is a microalgae.

12. The method according to claim 11 where the biological system is a Bacillariophycae.

13. The method according to claim 11 where the biological system is a diatom.

14. The method according to claim 11 where the diatom is a Pennate diatom, a Centric diatom, or combinations thereof.

15. The method according to claim 11 where the diatom genus is selected from the group consisting of Nitzschia, Pinnularia, Cyclotella, and combinations thereof.

16. The method according to claim 1 further comprising treating biomass produced after cultivation with an oxidizing agent.

17. The method according to claim 1 further comprising post processing a primary or secondary material.

18. The method according to claim 17 comprising combining the primary or secondary material with at least one metal, metal oxide, mixed metal oxide, mixed metal-semimetal oxide, or combinations thereof.

19. The method according to claim 1 where the metal oxide is a nanostructured metal oxide.

20. The method according to claim 1 where the metal oxide has a biogenic microstructure.

21. The method according to claim 17 where the metal oxide is a composite phosphor material selected from the group consisting of ZnS:Mn/SiO₂:Ge, ZnS:Tb/SiO₂:Ge, ZnS:Sm,C/SiO₂:Ge, ZnS:TbOF/SiO₂:Ge, ZnS:Tm,F/SiO₂:Ge, CaS:Eu/SiO₂:Ge, CaS:Ce,Cl/SiO₂:Ge, CaSSe:Eu/SiO₂:Ge, SrS:Ce,F/SiO₂:Ge, SrS:Ce,Mn,Cl/SiO₂:Ge, SrS:Ce/SiO₂:Ge, SrS:Ce/SiO₂:Ge, Eu/SiO₂:Ge, ZnO:Ga/SiO₂:Ge, ZnO:Al/SiO₂:Ge, ZnO:Cu/SiO₂:Ge, ZnSe:Cu/SiO₂:Ge, ZnSe:Cu,Cl/SiO₂:Ge, ZnSe:Mn/SiO₂:Ge, ZnTe:Mn/SiO₂:Ge, ZnTe:Eu/SiO₂:Ge, CdS:Ag/SiO₂:Ge, CdS:Te/SiO₂:Ge, CdS:ln/SiO₂:Ge, CdS:Te/SiO₂:Ge, CdSe:Cl/SiO₂:Ge, CdSe:Ag/SiO₂:Ge, CdTe:Ag/SiO₂:Ge, CdTe:Cl/SiO₂:Ge, CdO:Mn/SiO₂:Ge, CdO:Tm/SiO₂:Ge, CaS:Ce/SiO₂:Ge, CaS:Eu/SiO₂:Ge, CaS:Pb/SiO₂:Ge, CaS:Sm/SiO₂:Ge, CaSe:Ce/SiO₂:Ge, CaSe:Mn/SiO₂:Ge, CaSe:Tm/SiO₂:Ge, SrS:Ce/SiO₂:Ge, SrS:Eu/SiO₂:Ge, SrS:Mn/SiO₂:Ge, SrSe:Ba/SiO₂:Ge, SrSe:Ce/SiO₂:Ge, BaS:Au/SiO₂:Ge, BaS:Ce/SiO₂:Ge, BaS:Mn/SiO₂:Ge, BaSe:Eu/SiO₂:Ge, BaSe:Mn/SiO₂:Ge, LaPO₄:Eu/SiO₂:Ge, LaPO₄:Ce/SiO₂:Ge, CePO₄:Eu/SiO₂:Ge, CePO₄:Ce/SiO₂:Ge, GdPO₄:Tb/SiO₂:Ge, GdPO₄:Ce/SiO₂:Ge, YPO₄:Eu/SiO₂:Ge, YPO₄:Mn/SiO₂:Ge, YPO₄:V/SiO₂:Ge, ScBO₃:Tb/SiO₂:Ge, ScBO₃:Eu/SiO₂:Ge, ScBO₃:Ce/SiO₂:Ge, YBO₃:Eu/SiO₂:Ge, YBO₃:Ce/SiO₂:Ge, LaBO₃:Eu/SiO₂:Ge, LaBO₃:Mn/SiO₂:Ge, CeBO₃:Eu/SiO₂:Ge, CeBO₃:Tb/SiO₂:Ge, PbMoO₄:Eu/SiO₂:Ge, PbMoO₄:Mn/SiO₂:Ge, CaMoO₄:Eu/SiO₂:Ge, CaMoO₄:Ce/SiO₂:Ge, CdMoO₄:Eu/SiO₂:Ge, CdMoO₄:Mn/SiO₂:Ge, ZnMoO₄:Eu/SiO₂:Ge, ZnMoO₄:Ce/SiO₂:Ge, CdMoO₄:Mn/SiO₂:Ge, BaMoO₄:Mn/SiO₂:Ge, BaMoO₄:Eu/SiO₂:Ge, BaMoO₄:Tb/SiO₂:Ge, Gd₂(MoO₄):Mn/SiO₂:Ge, Gd₂(MoO₄):Tb/SiO₂:Ge, YVO₄:Eu/SiO₂:Ge, YVO₄:Dy/SiO₂:Ge, YPO₄:Eu/SiO₂:Ge, YPO₄:Mn/SiO₂:Ge, YPO₄:Mn,Th/SiO₂:Ge, CaWO₄:Sml/SiO₂:Ge, CaWO₄:Pb/SiO₂:Ge, PbWO₄:Tb/SiO₂:Ge, PbWO₄:Ce/SiO₂:Ge., and any and all combinations thereof.

22. The method according to claim 17 where the metal oxide is a silicate-based phosphor material selected from the group consisting of CaSiO₃:Ce, CaSiO₃:Eu, CaSiO₃:Pb, CaSiO₃:Ti, CaSiO₃:Pb,Mn, Be₂SiO₄:Mn, Mg₂SiO₄:Mn, Zn₂SiO₄:Mn, Zn₂SiO₄:Mn,P, Zn₂SiO₄:Mn,As, Zn₂SiO₄:Ti, (Zn+Be)₂SiO₄:Mn, Sr₂SiO₄:Eu, SrBaSiO₄:Eu, Ba₂SiO₄:Eu, Ba₂SiO₄:Ce,Li,Mn, BaSi₂₅:Eu, BaSi₂₅:Pb, Y₂SiO₅:Ce, CaMgSi₂O₆:Eu, CaMgSi₂O₆:Eu,Mn, Ca₂MgSi₂O₇:Eu, Ca₂MgSi₂O₇, Ca₂MgSi₂O₇:Eu,Mn, Sr₂MgSi₂O₇:Eu, Ba₂MgSi₂O₇:Eu, BaMg₂Si₂O₇:Eu, BaSrMgSi₂O₇:Eu, Ba₂Li₂Si₂O₇:Eu, Ba₂MgSi₂O₇:Sn, Ba₂MgSi₂O₇:Sn,Mn, MgSrBa₂Si₂O₇:Eu, MgBa₃Si₂O₈:Eu, MgSr₃Si₂O₈:Eu,Mn, Sr₃MgSi₂O₈:Eu, Ca₅B₂SiO₁₀:Eu, Ca₃Al₂Si₃O₁₂:Eu, LiCeBa₄Si₄O₁₄:Mn, LiCeSrBa₃Si₄O₁₄:Mn, and any and all combinations thereof.

23. The method according to claim 1, further comprising:
cultivating a diatom in the presence of a soluble source of silicon to substantial silicon starvation to produce a silicon-starved diatom;
cultivating the silicon-starved diatom for at least one generation in the presence of a soluble source of the substrate to produce a biomass; and
isolating the metal oxide from the biomass.

24. The method according to claim 23 where isolating comprises treating the biomass with an oxidizing agent to substantially oxidize organic material.

25. The method according to claim 23 where the metal oxide is a mixed metal oxide, a mixed semimetal oxide, a mixed metal-semimetal metal oxide, and any and all combinations thereof.

26. The method according to claim 23 where the metal oxide is photoluminescent.

27. The method according to claim 21 where the metal oxide is a nanostructured metal oxide.

28. The method according to claim 33 where the metal oxide has a biogenic microstructure.

29. The method according to claim 25 where the material is a Si—Ge oxide.

30. The method according to claim 25 where the substrate is a Group IIIa, IVa and/or Group IVb metal oxide.

31. The method according to claim 25 where the diatom is a Pennate diatom, a Centric diatom, or combinations thereof.

32. The method according to claim 25 where the diatom is selected from the group consisting of Nitzschia, Pinnularia, Cyclotella, and combinations thereof.

33. The method according to claim 25 further comprising post processing a primary or secondary material.

34. The method according to claim 33 where the metal oxide is a composite phosphor material selected from the group consisting of ZnS:Mn/SiO₂:Ge, ZnS:Tb/SiO₂:Ge, ZnS:Sm,Cl/SiO₂:Ge, ZnS:TbOF/SiO₂:Ge, ZnS:Tm,F/SiO₂:Ge, CaS:Eu/SiO₂:Ge, CaS:Ce,Cl/SiO₂:Ge, CaSSe:Eu/SiO₂:Ge, SrS:Ce,F/SiO₂:Ge, SrS:Ce,Mn,Cl/SiO₂:Ge, SrS:Ce/SiO₂:Ge, SrS:Ce/SiO₂:Ge, Eu/SiO₂:Ge, ZnO:Ga/SiO₂:Ge, ZnO:Al/SiO₂:Ge, ZnO:Cu/SiO₂:Ge, ZnSe:Cu/SiO₂:Ge, ZnSe:Cu,Cl/SiO₂:Ge, ZnSe:Mn/SiO₂:Ge, ZnTe:Mn/SiO₂:Ge, ZnTe:Eu/SiO₂:Ge, CdS:Ag/SiO₂:Ge, CdS:Te/SiO₂:Ge, CdS:In/SiO₂:Ge, CdS:Te/SiO₂:Ge, CdSe:Cl/SiO₂:Ge, CdSe:Ag/SiO₂:Ge, CdTe:Ag/SiO₂:Ge, CdTe:Cl/SiO₂:Ge, CdO:Mn/SiO₂:Ge, CdO:Tm/SiO₂:Ge, CaS:Ce/SiO₂:Ge, CaS:Eu/SiO₂:Ge, CaS:Pb/SiO₂:Ge, CaS:Sm/SiO₂:Ge, CaSe:Ce/SiO₂:Ge, CaSe:Mn/SiO₂:Ge, CaSe:Tm/SiO₂:Ge, SrS:Ce/SiO₂:Ge, SrS:Eu/SiO₂:Ge, SrS:Mn/SiO₂:Ge, SrSe:Ba/SiO₂:Ge, SrSe:Ce/SiO₂:Ge, BaS:Au/SiO₂:Ge, BaS:Ce/SiO₂:Ge, BaS:Mn/SiO₂:Ge, BaSe:Eu/SiO₂:Ge, BaSe:Mn/SiO₂:Ge, LaPO₄:Eu/SiO₂:Ge, LaPO₄:Ce/SiO₂:Ge, CePO₄:Eu/SiO₂:Ge, CePO₄:Ce/SiO₂:Ge, GdPO₄:Tb/SiO₂:Ge, GdPO₄:Ce/SiO₂:Ge, YPO₄:Eu/SiO₂:Ge, YPO₄:Mn/SiO₂:Ge, YPO₄:V/SiO₂:Ge, ScBO₃:Tb/SiO₂:Ge, ScBO₃:Eu/SiO₂:Ge, ScBO₃:Ce/SiO₂:Ge, YBO₃:Eu/SiO₂:Ge, YBO₃:Ce/SiO₂:Ge, LaBO₃:Eu/SiO₂:Ge, LaBO₃:Mn/SiO₂:Ge, CeBO₃:EU/SiO₂:Ge, CeBO₃:Tb/SiO₂:Ge, PbMoO₄:Eu/SiO₂:Ge, PbMoO₄:Mn/SiO₂:Ge, CaMoO₄:Eu/SiO₂:Ge, CaMoO₄:Ce/SiO₂:Ge, CdMoO₄:Eu/SiO₂:Ge, CdMoO₄:Mn/SiO₂:Ge, ZnMoO₄:Eu/SiO₂:Ge, ZnMoO₄:Ce/SiO₂:Ge, CdMoO₄:Mn/SiO₂:Ge, BaMoO₄:Mn/SiO₂:Ge, BaMoO₄:Eu/SiO₂:Ge, BaMoO₄:Tb/SiO₂:Ge, Gd₂(MoO₄):Mn/SiO₂:Ge, Gd₂(MoO₄):Tb/SiO₂:Ge, YVO₄:Eu/SiO₂:Ge, YVO₄:Dy/SiO₂:Ge, YPO₄:Eu/SiO₂:Ge, YPO₄:Mn/SiO₂:Ge, YPO₄:Mn,Th/SiO₂:Ge, CaWO₄:Sm/SiO₂:Ge, CaWO₄:Pb/SiO₂:Ge. PbWO₄:Tb/SiO₂:Ge, PbWO₄:Ce/SiO₂:Ge, and any and all combinations thereof.

35. The method according to claim 33 where the metal oxide is a silicate-based phosphor material selected from the group consisting of CaSiO₃:Ce, CaSiO₃:Eu, CaSiO₃:Pb, CaSiO₃:Ti, CaSiO₃:Pb,Mn, Be₂SiO₄:Mn, Mg₂SiO₄:Mn, Zn₂SiO₄:Mn, Zn₂SiO₄:Mn,P, Zn₂SiO₄:Mn,As, Zn₂SiO₄:Ti, (Zn+Be)₂SiO₄:Mn, Sr₂SiO₄:Eu, SrBaSiO₄:Eu, Ba₂SiO₄:Eu, Ba₂SiO₄:Ce,Li,Mn, BaSi₂₅:Eu, BaSi₂₅:Pb, Y₂SiO₅:Ce, CaMgSi₂O₆:Eu, CaMgSi₂O₆:Eu,Mn, Ca₂MgSi₂O₇:Eu, Ca₂MgSi₂O₇, Ca₂MgSi₂O₇:Eu,Mn, Sr₂MgSi₂O₇:Eu, Ba₂MgSi₂O₇:Eu, BaMg₂Si₂O₇:Eu, BaSrMgSi₂O₇:Eu, Ba₂Li₂Si₂O₇:Eu, Ba₂MgSi₂₀₇:Sn, Ba₂MgSi₂₀₇:Sn,Mn, MgSrBa₂Si₂O₇:Eu, MgBa₃Si₂O₈:Eu, MgSr₃Si₂O₈:Eu,Mn, Sr₃MgSi₂O₈:Eu, Ca₅B₂SiO10:Eu, Ca₃Al2Si₃O₁₂:Eu, LiCeBa₄Si₄O₁₄:Mn, LiCeSrBa₃Si₄O₁₄:Mn, and any and all combinations thereof.

36. The method according to claim 1 where the metal oxide is a Si—Ge oxide photoluminescent material, the method further comprising:
cultivating a diatom selected from a genus of Nitzschia, Pinnularia, Cyclotella, and combinations thereof to substantial silicon starvation in the presence of a source of soluble silicon to produce a silicon starved diatom;
cultivating the silicon-starved diatom for at least one cell division and for a period of greater than 24 hours in the presence of an effective concentration of a soluble source of germanium to produce a biomass; and
chemically oxidizing the biomass to produce the Si—Ge oxide photoluminescent material.

37. The method according to claim 36 where the metal oxide is a composite phosphor material selected from the group consisting of ZnS:Mn/SiO₂:Ge, ZnS:Tb/SiO₂:Ge, ZnS:Sm,Cl/SiO₂:Ge, ZnS:TbOF/SiO₂:Ge, ZnS:Tm,F/SiO₂:Ge, CaS:Eu/SiO₂:Ge, CaS:Ce,Cl/SiO₂:Ge, CaSSe:Eu/SiO₂:Ge, SrS:Ce,F/SiO₂:Ge, SrS:Ce,Mn,Cl/SiO₂:Ge, SrS:Ce/SiO₂:Ge, SrS:Ce/SiO₂:Ge, Eu/SiO₂:Ge, ZnO:Ga/SiO₂:Ge, ZnO:Al/SiO₂:Ge, ZnO:Cu/SiO₂:Ge, ZnSe:Cu/SiO₂:Ge, ZnSe:Cu,Cl/SiO₂:Ge, ZnSe:Mn/SiO₂:Ge, ZnTe:Mn/SiO₂:Ge, ZnTe:Eu/SiO₂:Ge, CdS:Ag/SiO₂:Ge, CdS:Te/SiO₂:Ge, CdS:In/SiO₂:Ge, CdS:Te/SiO₂:Ge, CdSe:Cl/SiO₂:Ge, CdSe:Ag/SiO₂:Ge, CdTe:Ag/SiO₂:Ge, CdTe:Cl/SiO₂:Ge, CdO:Mn/SiO₂:Ge, CdO:Tm/SiO₂:Ge, CaS:Ce/SiO₂:Ge, CaS:Eu/SiO₂:Ge, CaS:Pb/SiO₂:Ge, CaS:Sm/SiO₂:Ge, CaSe:Ce/SiO₂:Ge, CaSe:Mn/SiO₂:Ge, CaSe:Tm/SiO₂:Ge, SrS:Ce/SiO₂:Ge, SrS:Eu/SiO₂:Ge, SrS:Mn/SiO₂:Ge, SrSe:Ba/SiO₂:Ge, SrSe:Ce/SiO₂:Ge, BaS:Au/SiO₂:Ge, BaS:Ce/SiO₂:Ge, BaS:Mn/SiO₂:Ge, BaSe:Eu/SiO₂:Ge, BaSe:Mn/SiO₂:Ge, LaPO₄:Eu/SiO₂:Ge, LaPO₄:Ce/SiO₂:Ge, CePO₄:Eu/SiO₂:Ge, CePO₄:Ce/SiO₂:Ge, GdPO₄:Tb/SiO₂:Ge, GdPO₄:Ce/SiO₂:Ge, YPO₄:Eu/SiO₂:Ge, YPO₄:Mn/SiO₂:Ge, YPO₄:V/SiO₂:Ge, ScBO₃:Tb/SiO₂:Ge, ScBO₃:Eu/SiO₂:Ge, ScBO₃:Ce/SiO₂:Ge, YBO₃:Eu/SiO₂:Ge, YBO₃:Ce/SiO₂:Ge, LaBO₃:Eu/SiO₂:Ge, LaBO₃:Mn/SiO₂:Ge, CeBO₃:Eu/SiO₂:Ge, CeBO₃:Tb/SiO₂:Ge, PbMoO₄:Eu/SiO₂:Ge, PbMoO₄:Mn/SiO₂:Ge, CaMoO₄:Eu/SiO₂:Ge, CaMoO₄:Ce/SiO₂:Ge, CdMoO₄:Eu/SiO₂:Ge, CdMoO₄:Mn/SiO₂:Ge, ZnMoO₄:Eu/SiO₂:Ge, ZnMoO₄:Ce/SiO₂:Ge, CdMoO₄:Mn/SiO₂:Ge, BaMoO₄:Mn/SiO₂:Ge, BaMoO₄:Eu/SiO₂:Ge, BaMoO₄:Tb/SiO₂:Ge, Gd₂(MoO₄):Mn/SiO₂:Ge, Gd₂(MoO₄):Tb/SiO₂:Ge, YVO₄:Eu/SiO₂:Ge, YVO₄:Dy/SiO₂:Ge, YPO₄:Eu/SiO₂:Ge, YPO₄:Mn/SiO₂:Ge, YPO₄:Mn,Th/SiO₂:Ge, CaWO₄:Srn/SiO₂:Ge, CaWO₄:Pb/SiO₂:Ge, PbWO₄:Tb/SiO₂:Ge, PbWO₄:Ce/SiO₂:Ge, and any and all combinations thereof.

38. The method according to claim 36, where cultivating comprises cultivating a diatom selected from Nitzschia acicularis, Nitzschia alba, Nitzschia alexandrina, Nitzschia amphibian, Nitzschia apiculata, Nitzschia closterium, Nitzschia communis, Nitzschia compressa, Nitzschia dissipata, Nitzschia distans, Nitzschia filiformis, Nitzschia fonticola, Nitzschia frustulum, Nitzschia fusiformis, Nitzschia inconspicua, Nitzschia laevis, Nitzschia lecointei, Nitzschia linearis, Nitzschia longissima, Nitzschia navis-varingica, Nitzschia palea, Nitzschia paleacea, Nitzschia paleafonnis, Nitzschia pellucida, Nitzschia pusilla, Nitzschia sigma, Nitzschia supralitorea, Nitzschia thermalis, Nitzschia thermaloides, Nitzschia vitrea, Pinnularia quadratarea, Pinnularia rupestri, and combinations thereof to substantial silicon starvation in the presence of silicic acid to produce a silicon starved diatom.

39. The method according to claim 38 where chemically oxidizing comprises oxidizing the aqueous biomass with hydrogen peroxide to produce the Si—Ge oxide photoluminescent material.

40. The method according to claim 17 where the metal oxide is a secondary material, and the method further comprises combining the secondary material with a metal, a metal oxide, a mixed metal oxide, a semimetal, a semimetal oxide, a mixed semimetal oxide, a mixed metal-semimetal oxide, or combinations thereof.

41. The method according to claim 40 where the metal oxide is a nanostructured metal oxide, where the metal oxide has a biogenic microstructure, or both.

42. The method according to claim 40 where the metal oxide is a composite phosphor material selected from the group consisting of ZnS:Mn/SiO₂:Ge, ZnS:Tb/SiO₂:Ge, ZnS:Sm,Cl/SiO₂:Ge, ZnS:TbOF/SiO₂:Ge, ZnS:Tm,F/SiO₂:Ge, CaS:Eu/SiO₂:Ge, CaS:Ce,Cl/SiO₂:Ge, CaSSe:Eu/SiO₂:Ge, SrS:Ce,F/SiO₂:Ge, SrS:Ce,Mn,Cl/SiO₂:Ge, SrS:Ce/SiO₂:Ge, SrS:Ce/SiO₂:Ge, Eu/SiO₂:Ge, ZnO:Ga/SiO₂:Ge, ZnO:Al/SiO₂:Ge, ZnO:Cu/SiO₂:Ge, ZnSe:Cu/SiO₂:Ge, ZnSe:Cu,Cl/SiO₂:Ge, ZnSe:Mn/SiO₂:Ge, ZnTe:Mn/SiO₂:Ge, ZnTe:Eu/SiO₂:Ge, CdS:Ag/SiO₂:Ge, CdS:Te/SiO₂:Ge, CdS:In/SiO₂:Ge, CdS:Te/SiO₂:Ge, CdSe:ClI/SiO₂:Ge, CdSe:Ag/SiO₂:Ge, CdTe:Ag/SiO₂:Ge, CdTe:Cl/SiO₂:Ge, CdO:Mn/SiO₂:Ge, CdO:Tm/SiO₂:Ge, CaS:Ce/SiO₂:Ge, CaS:Eu/SiO₂:Ge, CaS:Pb/SiO₂:Ge, CaS:Sm/SiO₂:Ge, CaSe:Ce/SiO₂:Ge, CaSe:Mn/SiO₂:Ge, CaSe:Tm/SiO₂:Ge, SrS:Ce/SiO₂:Ge, SrS Eu/SiO₂:Ge, SrS:Mn/SiO₂:Ge, SrSe:Ba/SiO₂:Ge, SrSe:Ce/SiO₂:Ge, BaS:Au/SiO₂:Ge, BaS:Ce/SiO₂:Ge, BaS:Mn/SiO₂:Ge, BaSe:Eu/SiO₂:Ge, BaSe:Mn/SiO₂:Ge, LaPO₄:Eu/SiO₂:Ge, LaPO₄:Ce/SiO₂:Ge, CePO₄:Eu/SiO₂:Ge, CePO₄:Ce/SiO₂:Ge, GdPO₄:Tb/SiO₂:Ge, GdPO₄:Ce/SiO₂:Ge, YPO₄:Eu/SiO₂:Ge, YPO₄:Mn/SiO₂:Ge, YPO₄:V/SiO₂:Ge, ScBO₃:Tb/SiO₂:Ge, ScBO₃:Eu/SiO₂:Ge, ScBO₃:Ce/SiO₂:Ge, YBO₃:Eu/SiO₂:Ge, YBO₃:Ce/SiO₂:Ge, LaBO₃:Eu/SiO₂:Ge, LaBO₃:Mn/SiO₂:Ge, CeBO₃:Eu/SiO₂:Ge, CeBO₃:Tb/SiO₂:Ge, PbMoO₄:Eu/SiO₂:Ge, PbMoO₄:Mn/SiO₂:Ge, CaMoO₄:Eu/SiO₂:Ge, CaMoO₄:Ce/SiO₂:Ge, CdMoO₄:Eu/SiO₂:Ge, CdMoO₄:Mn/SiO₂:Ge, ZnMoO₄:Eu/SiO₂:Ge, ZnMoO₄:Ce/SiO₂:Ge, CdMoO₄:Mn/SiO₂:Ge, BaMoO₄:Mn/SiO₂:Ge, BaMoO₄:Eu/SiO₂:Ge, BaMoO₄:Tb/SiO₂:Ge, Gd₂(MoO₄):Mn/SiO₂:Ge, Gd₂(MoO₄):Tb/SiO₂:Ge, YVO₄:Eu/SiO₂:Ge, YVO₄:Dy/SiO₂:Ge, YPO₄:Eu/SiO₂:Ge, YPO₄:Mn/SiO₂:Ge, YPO₄:Mn,Th/SiO₂:Ge, CaWO₄:Sn/SiO₂:Ge, CaWO₄:Pb/SiO₂:Ge, PbWO₄:Tb/SiO₂:Ge, PbWO₄:Ce/SiO₂:Ge, and any and all combinations thereof.

43. The method according to claim 40 where the metal oxide is a silicate-based phosphor material selected from the group consisting of CaSiO₃:Ce, CaSiO₃:Eu, CaSiO₃:Pb, CaSiO₃:Ti, CaSiO₃:Pb,Mn, Be₂SiO₄:Mn, Mg₂SiO₄:Mn, Zn₂SiO₄:Mn, Zn₂SiO₄:Mn,P, Zn₂SiO₄:Mn,As, Zn₂SiO₄:Ti, (Zn+Be)₂SiO₄:Mn, Sr₂SiO₄:Eu, SrBaSiO₄:Eu, Ba₂SiO₄:Eu, Ba₂SiO₄:Ce,Li,Mn, BaSi₂₅:Eu, BaSi₂₅:Pb, Y₂SiO₅:Ce, CaMgSi₂O₆:Eu, CaMgSi₂O₆:Eu,Mn, Ca₂MgSi₂O₇:Eu, Ca₂MgSi₂O₇, Ca₂MgSi₂O₇:Eu,Mn, Sr₂MgSi₂O₇:Eu, Ba₂MgSi₂O₇:Eu, BaMg₂Si₂O₇:Eu, BaSrMgSi₂O₇:Eu, Ba₂Li₂Si₂O₇:Eu, Ba₂MgSi₂O₇:Sn, Ba₂MgSi₂O₇:Sn,Mn, MgSrBa₂Si₂O₇:Eu, MgBa₃Si₂O₈:Eu, MgSr₃Si₂O₈:Eu,Mn, Sr₃MgSi₂O₈:Eu, Ca₅B₂SiO10:Eu, Ca₃Al₂Si₃O₁₂:Eu, LiCeBa₄Si₄O₁₄:Mn, LiCeSrBa₃Si₄O₁₄:Mn, and any and all combinations thereof.

44. A metal oxide produced according to the method of claim 1.

45. A metal oxide produced according to the method of claim 23.

46. A metal oxide produced according to the method of claim 36.

47. A method for making an optoelectronic device, comprising:
making a primary or secondary metal oxide biologically; and
making an optoelectronic device comprising the metal oxide.

48. The method according to claim 47 where the device is selected from memory devices, data storage devices, capacitors, detectors, sensors, lasers, emitters, light-emitting diodes, optical computing architectures, photonics and photovoltaics.

49. A method for administering an active agent, comprising:
making a primary or secondary luminescent metal oxide biologically;
forming a composition comprising at least one active agent and the metal oxide; and
administering the composition to a subject.

50. The method according to claim 49 further comprising inducing active agent release after administering the composition.

51. The method according to claim 49 where the active agent is selected from the group consisting of nucleic acids, proteins, naturally occurring organic compounds, synthetic and semi-synthetic compounds, and combinations thereof. More particularly, the diagnostic or therapeutic agent may be an AIDS adjunct agent, alcohol abuse preparation, Alzheimer's disease management agent, amyotrophic lateral sclerosis therapeutic agent, analgesic, anesthetic, antacid, antiarythmic, antibiotic, anticonvulsant, antidepressant, antidiabetic agent, antiemetic, antidote, antifibrosis therapeutic agent, antifungal, antihistamine, antihypertensive, anti-infective agent, antimicrobial, antineoplastic, antipsychotic, antiparkinsonian agent, antirheumatic agent, appetite stimulant, appetite suppressant, biological response modifier, biological, blood modifier, bone metabolism regulator, cardioprotective agent, cardiovascular agent, central nervous system stimulant, cholinesterase inhibitor, contraceptive, cystic fibrosis management agent, deodorant, diagnostic, dietary supplement, diuretic, dopamine receptor agonist, endometriosis management agent, enzyme, erectile dysfunction therapeutic, fatty acid, gastrointestinal agent, Gaucher's disease management agent, gout preparation, homeopathic remedy, hormone, hypercalcemia management agent, hypnotic, hypocalcemia management agent, immunomodulator, immunosuppressive, ion exchange resin, levocamitine deficiency management agent, mast cell stabilizer, migraine preparation, motion sickness product, multiple sclerosis management agent, muscle relaxant, narcotic detoxification agent, narcotic, nucleoside analog, non-steroidal anti-inflammatory drug, obesity management agent, osteoporosis preparation, oxytocic, parasympatholytic, parasympathomimetic, phosphate binder, porphyria agent, psychotherapeutic agent, radio-opaque agent, psychotropic, sclerosing agent, sedative, sickle cell anemia management agent, smoking cessation aid, steroid, stimulant, sympatholytic, sympathomimetic, Tourette's syndrome agent, tremor preparation, urinary tract agent, vaginal preparation, vasodilator, vertigo agent, weight loss agent, Wilson's disease management agent, and mixtures thereof.

52. The method according to claim 49 where the active agent is selected from the group consisting of abacavir sulfate, abacavir sulfate/lamivudine/zidovudine, acetazolamide, acyclovir, albendazole, albuterol, aldactone, allopurinol BP, amoxicillin, amoxicillin/clavulanate potassium, amprenavir, atovaquone, atovaquone and proguanil hydrochloride, atracurium besylate, beclomethasone dipropionate, berlactone betamethasone valerate, bupropion hydrochloride, bupropion hydrochloride SR, captopril, carvedilol, caspofingin acetate, cefazolin, ceftazidime, cefuroxime (no sulfate), chlorambucil, chlorpromazine, cimetidine, cimetidine hydrochloride, cisatracurium besilate, clobetasol propionate, co-trimoxazole, colfosceril palmitate, dextroamphetamie sulfate, digoxin, enalapril maleate, epoprostenol, esomepraxole magnesium, fexofenadine, fluticasone propionate, furosemide, gancyclovir, hydrochlorothiazide/triamterene, lamivudine, lamotrigine, lithium carbonate, losartan potassium, melphalan, mercaptopurine, mesalazine, metformin, methyldopa, minocycline, mupirocin calcium cream, nabumetone, naratriptan, omeprazole, ondansetron hydrochloride, orli stat (or a pharmaceutically acceptable salt thereof), ovine, oxiconazole nitrate, paroxetine hydrochloride, prochlorperazine, procyclidine hydrochloride, pyrimethamine, ranitidine bismuth citrate, ranitidine hydrochloride, rofecoxib, ropinirole hydrochloride, rosiglitazone maleate, salmeterol xinafoate, salmeterol, selegiline, fluticasone propionate, sterile ticarcillin disodium/clavulanate potassium, simvastatin, spironolactone, succinylcholine chloride, sumatriptan, thioguanine, tirofiban HCI, topotecan hydrochloride, tranylcypromine sulfate, trifluoperazine hydrochloride, valacyclovir hydrochloride, vinorelbine, zanamivir, zidovudine, zidovudine, lamivudine, and combinations thereof.

53. The method according to claim 49 where the composition comprises a light sensitive liposome.

54. A method for imaging tissue, comprising:
making a primary or secondary metal oxide biologically;
administering the metal oxide to a subject; and
imaging the subject by luminescence from the metal oxide.

55. A method for surface functionalizing diatom biosilica, comprising:
providing diatom biosilica having plural Si—OH groups as a first functional group; and
functionalizing at least a portion of the Si—OH group to produce functionalized biocilica having at least one second functional group.

56. The method according to claim 55 where the Si—OH groups are surface functionalized with amine groups.

57. The method according to claim 56 further comprising coupling desired molecules to the at least one second functional group.

58. The method according to claim 57 further comprising covalently attaching N-terminal biomolecules to the second functional group selected from peptides, proteins, streptavidin-biotin, antibodies, and combinations thereof.
